SEQUENCE LISTING (61 sequences)

SEQ ID NO: 1 - DNA, 7936 bp, Arabidopsis thaliana
tccatggcccaaaagctttttcccggtgtacttggacttttcctcactggtgtgcgttagagtagcaagaatgtattgttgactattcttttctctttttttaaatacctttttgttagtttttgctgttggaaaaatatcaaatgtaaaaaaaacactgagatttgagtggttgaatgtccaatgaaaaagcatcgtcggcataaacaaaaattccggcgaaatctaaggttagttcaaagtatcaaagcgcgaaatcatgaccaattgtgaaaaggatgaggaattcgtgtgcatcagctgcgtagaagaggtacggtactctttcgtgagccacctctctgaagctctccgtcgaaaaggcataaataatgtggtcgtagatgtagatatcgatgatctgcttttcaaggagtctcaggcaaagatcgagaaagctggggtttctgtgatggttttacccggaaactgtgacccttccgaggtatggcttgacaagttcgccaaggttctcgagtgccaaaggaacaacaaggaccaggcggtggtttcagtgttgtacggtgacagtctattacgggaccaatggcttagcgagctggatttcagaggcttatcacgaattcaccaatccaggttttcgctttattctactctctgctttttttttccttatgttgaaaattggtaaaactttttggatggctagtgtagtgttttcaatatttgatgaaatatggtgcaggaaggaatgtagtgactctatacttgtagaagagattgtgagagatgtgtacgagacgcacttttatgttggacgaattggaatctattcgaagctgctggagattgaaaacatggttaacaagcaaccgataggcatccgttgtgttggaatttggggtatgcctggcataggaaagacaacacttgctaaagcagtctttgaccaaatgtctagcgcctttgatgcttcttgttttatcgaagactatgacaaatcaattcatgagaagggtctttattgtttgctggaggaacaacttttgccgggtaatgatgcaaccattatgaaactgagctcgctcagagacagattgaacagtaagagagttcttgttgttctcgatgacgtgtgcaatgctctggttgcagagtcttttctcgaggggtttgactggctaggacccggaagcctgatcatcataacctctagagataaacaagtgtttcgcctttgcggaatcaatcaaatatatgaggtccagggtttaaatgagaaagaggctcgtcaacttttcttgctgtctgcgtctataatggaggatatgggagagcagaatctccatgagttgtcagtgagagtaataagttacgctaatggaaacccgttagctatcagtgtttatggaagagagctgaaaggtaagaaaaaactctcagaaatggagactgcattcctcaaactcaagcgacgtcctccatttaagattgtcgatgcatttaaaagcagctacgacacactcagtgacaacgaaaagaacatttttttggacatagcttgttttttccagggagaaaatgtcaactacgtgatacaactgcttgagggttgtggtttctttccacatgttgaaattgatgtccttgttgacaagtgtctggtgactatttcagaaaaccgagtttggttgcataagctgacccaggatatcggccgagaaatcataaatggagaaacagtacagatcgagaggcgcagaagactgtgggaaccttggagcatcaaatatttattagaatataatgaacacaaagcaaatggagaacctaaaacaaccttcaaacgtgctcaggtttgttatattttgatagtttcatatttattctactttatgagccaacggcctaatatatccacttccaatattttcagggctctgaagagatcgaaggcctgtttctagacacatcaaacttaagatttgatctgcagccctctgcctttaagaatatgttgaaccttagattgctcaaaatttattgttccaatcctgaagtccatcctgtaatcaatttcccaacaggctctctgcattctcttcctaatgagctaagactcctccattgggagaactatcctctgaaatctttgcctcagaattttgatcctaggcaccttgtcgaaatcaacatgccgtatagtcaacttcagaaactttggggtggaaccaaggtaagcaaactctgcgtctttcacatctatcattacttgaaattttttgttgttattcatttagcttgcttttgtttttgtcttctcagaacctggagatgttgaggacgatcaggctttgccattcccagcatctagttgatatcgatgatctcttaaaagctgaaaatcttgaggtaattgatctccaaggttgtacgagactgcagaatttcccagccgcaggtcgattgctacgtctacgagttgtaaatctctcaggttgcataaagattaaaagtgtcctagaaattccaccaaatattgagaaactacatctacagggaactggcatattagcattaccagtttccactgttaagccaaaccatagagagcttgtgaattttctaacagaaattccgggtctttcagaggcctcgaaacttgagcgtttaacaagtctgctggaatctaactcatcttgtcaagatcttgggaagcttatttgcttggagctgaaagattgctcttgtttgcagagtctgccaaacatggctaatttagatcttaatgttcttgatctctcgggttgctcaagtcttaattctattcagggtttccctcgttttctgaaacagttatatcttggtggcactgcaataagagaagtgccacaacttcctcaaagtctagaaatcttgaatgcacatggatcttgtttgcgaagtctgccaaacatggctaatttagaatttctcaaagttcttgatctctctggttgctcagagctcgagactattcagggttttcctcggaacctaaaagagttatattttgctggcactacgttaagagaagtgccccaacttcctttaagcctagaggtcttgaatgcacatggttctgactcggagaagcttcctatgcattacaagttcaacaattttttcgatctatctcaacaagtggtcaacgattttttcttgaaagcgctgacttatgtaaaacacataccaagagggtatacgcaggtaatactctctctccttctgcctctctcttgtctacttacctctccattagctctctttctttccatcaccccctcaatcctttctctttctctgtccgcctatgtctcccgctctctatagtgcctgttatgtacacccaaccacaccctcctcatatcatatctgacttatttgtattgcaacaggaactcatcaacaaagctccgactttcagcttcagtgcgccctcacatacaaatcaaaacgccacatttgatctgcaaccaggatcttctgtaatgacacgactaaatcattcatggaggaacacgcttgtgggatttggtatgctggtggaagttgcatttcccgaggactactgtgatgctacagatgttggcataagttgtgtttgcagatggagcaacaaagaaggccgctcttgtaggatagaaagaaattttcattgttgggcaccagggaaagttgttccaaaagttcgaaaggatcatacgtttgtctttagtgatgtcaacatgcgcccaagtaccggtgaaggaaatgaccctgatatctgggctggattagttgtatttgagttctttcctatcaatcagcagacaaagtgtctaaatgataggttcacagtgacaagatgtggagtccgtgtaataaatgttgcaactggcaatacaagtcttgagaacatatcactagttttgtctttggatccagtggaggtttctggttatgaagtattgagagtcagctatgatgatttacaggagatggataaagttctatttctttacatagcgtctttgttcaatgacgaggatgttgattttgtggcaccacttattgccggtattgacttggatgttagctctgggctcaaggtcttagccgatgtgtctctcataagtgtatcatccaatggggaaatagtgatgcatagtttgcaaagacaaatgggcaaagaaatcctccatggacaatccatgctgctgtctgattgtgagagttccatgaccgagaatttgtctgacgtaccaaaaaagtaagttctctttttctatcagcttcatatacaaccgtagacttaaataaatttgaaaatatttgagtacatataatcacattaggtgcaatgaaaaaacgtgtaatttattagattgaactaaagtttacaaaaagccattagagatgtatcagtatgctaccttttccaagttgcttaataacataggtttttcatgtttgtactttttagattaatttaaaaatccgtttttttctccattcaagattttagaaaagtttattttttatttaatgaaatattagatttgttgtgaaaaagacattaatttacaatcagaggtaaaatctctagagacatttggcattttcgctcggctttctttgttaaatccttttttcgtgaggattttagaaaagttgattgtgagctactcaccaaggtttcatctttttgaaaaatactcttacataagatgtgtcaaggaaatgttagtttatctaacaaaaacccacttctcaacgtttccatatctatgtatctttttactttcatgaaaccatgagagacatatatgcattatgaaattttctgaattcttacattccttatagaacttctctcccacccacaaagatttgggaatcattagtctttttttttttttttcaattttcaattgatgtttgcttttgttttacttcttattatcatgttaaaaacttttttgatgccataaaaataacctttatacgtatgttgtaatcatttttatggtaacatacattagatttttaaactttgccacaaaaaaaaaaaaaaattagatttttaaaattctaaaagtagagttaaactccgtgtcgatagcaaaagagggttacattacgtgttactttttactttgtgtgattaatctgcattttagaggtagagggtaaaattgcaaaaatttggcgaatggaatttttttttcttccacaaaaccttatcttaaaccgaactgatcatggcacacactaacattgatcacagaagtagctataaatcacaacatataatcacatgggtttcgatatatgttatatcgacgttggatgcagggagaagaaacatcgcgaaagtaaggtaaagaaagtggtttccataccggctatagacgagggagatctatggacttggcgaaagtacggtcaaaaagacatcttaggttctcgttttccaaggtacactcatgtatttttgtatatacatattcctatatttgtgtatatattatattctttacaaacataataaatagttatacatataaacaaattatttaaatccaacaaaaacaaaatagtattttacaataggaaagttttgtaacaacttgcaaaacaccattttttatacacaaacgtaatcatcctaaacttgctggtggcctggtaaaccttcatatgttgctggaaaagtgaatatttctaagaaaaaacaaaaattattaaccattgaaaaagtcacattaagcagaatctgcagactgcagctttgtttttccccttcactaacagtattgcagataaaatcattagcgtgaaactgtaaaatagaaagttcatttaattatctacggcgcaaattataagcctttaaattcttttttatgatgcagacttatcttataatcacaatattccatttgaaggatattatagatgtgtttttttttttttaatccagttgtgcgtaagtgatcaaattatggttagttatgacttttgatggtcacttgatgacttaattaattatttgttcctcaggggttactacaggtgcgcttacaagttcacgcatggttgtaaagctacaaaacaagtccaacggagcgagaccgattcaaacatgttagctattacttacctatctgagcataaccatccacggcccactaaacgcaaggctctcgctgactccactcgttccacttcctcctccatctgctcagccataactacctctgcctcatctagagtcttccaaaacaaagacgaaccaaatcaaccccacttgccttcctcctccactcctcctagaaacgcggctgtcttgtttaaaatgacggacatggaggagtttcaggacaatatggaggtggataatgacgtcgtagatacacgtacactggcattgtttccagagtttcaacatcagccggaggaagaagacccatggtcaacattcttcgatgattataatttttacttttgattgagctcactctcatcactgcaagacaagaaaaaataaccagtgagatccaaggatttatgtacacccaaggatttagtttttgcagtgtcaatgattttcagccaataataaatcattaatttcaattaccagtcctccccaacctgtaaacccatcatgcctccaacatacggtgatctcgacaaattcgaaagcctaataccaaggatacatgtatcacagtaggatttctcaatcctgatgcactcgaagaggaatagatcttaagaaatcattaagccttatgaaagcaaggaaagaaatggctgagatgtcttacgtacgttgattacaaatagaggcaatcaacacaacaattgttttgttgtgggttttgtgtcgatataccattgcggttaagagcctgccaaatgggtatgtaagctagagatctctctcatgtgtttattctctttgatttgtgtaaagctttgaactttcctattttcaataaaaccatagtctgaagaggtgaatttcgttatccctattttcaactaaagatgtagtatatgaaaattacctaaaatctttttcaaaatcctttcttaaaaggtttgataatttttgaataacaatggatttgatttgagtttcacaaatcattgattgaatagaagaaaaggatggtaaatgattttgaattattttacataaatcaataattaataacaggtgattaataaaaaagactcgaaatcttcaaagagcatctccataagagtatcttaacaataaaatgacattatttctatagaatttagttgtttaattaaatttaaatttgttacaaaattaacccaacaatcaagtgacatgggtaaaatagagtttctaaatcataagtttctcaaaagttacatgtgtaatataccggctatgctttggtttctgttgtctgtgaagatgatccttctggaaggtctcgaaggcttcctctggactccccttcgattcatgtttctgtattgacgcgatgtctagatcttccgtttgtgacatagtgaatgaattaagaaggggtttgtttctttcagtagtttaaggatgcgttttgtaacctcggaattagtttggactcggccacttgtgtatttctaagttcttagtatttgatctttggcttgctttgtggttacctttccttcaagctttaggattttagtagttgagactcttgcagaagtttaggattgattggattgcactcatggctccactgggtcgcgcgttaagtgtaaaggttcctaggacgggggttaagtgagagatttgatttgtcctctcttagtattttattttctgcttgaactttctgatatatctcctctatgtcttagatccttcttagtttgatccatt

SEQ ID NO: 2 - DNA, 259 bp, Arabidopsis thaliana
tccatggcccaaaagctttttcccggtgtacttggacttttcctcactggtgtgcgttagagtagcaagaatgtattgttgactattcttttctctttttttaaatacctttttgttagtttttgctgttggaaaaatatcaaatgtaaaaaaaacactgagatttgagtggttgaatgtccaatgaaaaagcatcgtcggcataaacaaaaattccggcgaaatctaaggttagttcaaagtatcaaagcgcgaaatc

SEQ ID NO: 3 - DNA, 1404 bp, Arabidopsis thaliana
ttgagctcactctcatcactgcaagacaagaaaaaataaccagtgagatccaaggatttatgtacacccaaggatttagtttttgcagtgtcaatgattttcagccaataataaatcattaatttcaattaccagtcctccccaacctgtaaacccatcatgcctccaacatacggtgatctcgacaaattcgaaagcctaataccaaggatacatgtatcacagtaggatttctcaatcctgatgcactcgaagaggaatagatcttaagaaatcattaagccttatgaaagcaaggaaagaaatggctgagatgtcttacgtacgttgattacaaatagaggcaatcaacacaacaattgttttgttgtgggttttgtgtcgatataccattgcggttaagagcctgccaaatgggtatgtaagctagagatctctctcatgtgtttattctctttgatttgtgtaaagctttgaactttcctattttcaataaaaccatagtctgaagaggtgaatttcgttatccctattttcaactaaagatgtagtatatgaaaattacctaaaatctttttcaaaatcctttcttaaaaggtttgataatttttgaataacaatggatttgatttgagtttcacaaatcattgattgaatagaagaaaaggatggtaaatgattttgaattattttacataaatcaataattaataacaggtgattaataaaaaagactcgaaatcttcaaagagcatctccataagagtatcttaacaataaaatgacattatttctatagaatttagttgtttaattaaatttaaatttgttacaaaattaacccaacaatcaagtgacatgggtaaaatagagtttctaaatcataagtttctcaaaagttacatgtgtaatataccggctatgctttggtttctgttgtctgtgaagatgatccttctggaaggtctcgaaggcttcctctggactccccttcgattcatgtttctgtattgacgcgatgtctagatcttccgtttgtgacatagtgaatgaattaagaaggggtttgtttctttcagtagtttaaggatgcgttttgtaacctcggaattagtttggactcggccacttgtgtatttctaagttcttagtatttgatctttggcttgctttgtggttacctttccttcaagctttaggattttagtagttgagactcttgcagaagtttaggattgattggattgcactcatggctccactgggtcgcgcgttaagtgtaaaggttcctaggacgggggttaagtgagagatttgatttgtcctctcttagtattttattttctgcttgaactttctgatatatctcctctatgtcttagatccttcttagtttgatccatt

SEQ ID NO: 4 - DNA, 4343 bp, Arabidopsis thaliana
gtccaatgaaaaagcatcgtcggcataaacaaaaattccggcgaaatctaaggttagttcaaagtatcaaagcgcgaaatcatgaccaattgtgaaaaggatgaggaattcgtgtgcatcagctgcgtagaagaggtacggtactctttcgtgagccacctctctgaagctctccgtcgaaaaggcataaataatgtggtcgtagatgtagatatcgatgatctgcttttcaaggagtctcaggcaaagatcgagaaagctggggtttctgtgatggttttacccggaaactgtgacccttccgaggtatggcttgacaagttcgccaaggttctcgagtgccaaaggaacaacaaggaccaggcggtggtttcagtgttgtacggtgacagtctattacgggaccaatggcttagcgagctggatttcagaggcttatcacgaattcaccaatccaggaaggaatgtagtgactctatacttgtagaagagattgtgagagatgtgtacgagacgcacttttatgttggacgaattggaatctattcgaagctgctggagattgaaaacatggttaacaagcaaccgataggcatccgttgtgttggaatttggggtatgcctggcataggaaagacaacacttgctaaagcagtctttgaccaaatgtctagcgcctttgatgcttcttgttttatcgaagactatgacaaatcaattcatgagaagggtctttattgtttgctggaggaacaacttttgccgggtaatgatgcaaccattatgaaactgagctcgctcagagacagattgaacagtaagagagttcttgttgttctcgatgacgtgtgcaatgctctggttgcagagtcttttctcgaggggtttgactggctaggacccggaagcctgatcatcataacctctagagataaacaagtgtttcgcctttgcggaatcaatcaaatatatgaggtccagggtttaaatgagaaagaggctcgtcaacttttcttgctgtctgcgtctataatggaggatatgggagagcagaatctccatgagttgtcagtgagagtaataagttacgctaatggaaacccgttagctatcagtgtttatggaagagagctgaaaggtaagaaaaaactctcagaaatggagactgcattcctcaaactcaagcgacgtcctccatttaagattgtcgatgcatttaaaagcagctacgacacactcagtgacaacgaaaagaacatttttttggacatagcttgttttttccagggagaaaatgtcaactacgtgatacaactgcttgagggttgtggtttctttccacatgttgaaattgatgtccttgttgacaagtgtctggtgactatttcagaaaaccgagtttggttgcataagctgacccaggatatcggccgagaaatcataaatggagaaacagtacagatcgagaggcgcagaagactgtgggaaccttggagcatcaaatatttattagaatataatgaacacaaagcaaatggagaacctaaaacaaccttcaaacgtgctcagggctctgaagagatcgaaggcctgtttctagacacatcaaacttaagatttgatctgcagccctctgcctttaagaatatgttgaaccttagattgctcaaaatttattgttccaatcctgaagtccatcctgtaatcaatttcccaacaggctctctgcattctcttcctaatgagctaagactcctccattgggagaactatcctctgaaatctttgcctcagaattttgatcctaggcaccttgtcgaaatcaacatgccgtatagtcaacttcagaaactttggggtggaaccaagaacctggagatgttgaggacgatcaggctttgccattcccagcatctagttgatatcgatgatctcttaaaagctgaaaatcttgaggtaattgatctccaaggttgtacgagactgcagaatttcccagccgcaggtcgattgctacgtctacgagttgtaaatctctcaggttgcataaagattaaaagtgtcctagaaattccaccaaatattgagaaactacatctacagggaactggcatattagcattaccagtttccactgttaagccaaaccatagagagcttgtgaattttctaacagaaattccgggtctttcagaggcctcgaaacttgagcgtttaacaagtctgctggaatctaactcatcttgtcaagatcttgggaagcttatttgcttggagctgaaagattgctcttgtttgcagagtctgccaaacatggctaatttagatcttaatgttcttgatctctcgggttgctcaagtcttaattctattcagggtttccctcgttttctgaaacagttatatcttggtggcactgcaataagagaagtgccacaacttcctcaaagtctagaaatcttgaatgcacatggatcttgtttgcgaagtctgccaaacatggctaatttagaatttctcaaagttcttgatctctctggttgctcagagctcgagactattcagggttttcctcggaacctaaaagagttatattttgctggcactacgttaagagaagtgccccaacttcctttaagcctagaggtcttgaatgcacatggttctgactcggagaagcttcctatgcattacaagttcaacaattttttcgatctatctcaacaagtggtcaacgattttttcttgaaagcgctgacttatgtaaaacacataccaagagggtatacgcaggaactcatcaacaaagctccgactttcagcttcagtgcgccctcacatacaaatcaaaacgccacatttgatctgcaaccaggatcttctgtaatgacacgactaaatcattcatggaggaacacgcttgtgggatttggtatgctggtggaagttgcatttcccgaggactactgtgatgctacagatgttggcataagttgtgtttgcagatggagcaacaaagaaggccgctcttgtaggatagaaagaaattttcattgttgggcaccagggaaagttgttccaaaagttcgaaaggatcatacgtttgtctttagtgatgtcaacatgcgcccaagtaccggtgaaggaaatgaccctgatatctgggctggattagttgtatttgagttctttcctatcaatcagcagacaaagtgtctaaatgataggttcacagtgacaagatgtggagtccgtgtaataaatgttgcaactggcaatacaagtcttgagaacatatcactagttttgtctttggatccagtggaggtttctggttatgaagtattgagagtcagctatgatgatttacaggagatggataaagttctatttctttacatagcgtctttgttcaatgacgaggatgttgattttgtggcaccacttattgccggtattgacttggatgttagctctgggctcaaggtcttagccgatgtgtctctcataagtgtatcatccaatggggaaatagtgatgcatagtttgcaaagacaaatgggcaaagaaatcctccatggacaatccatgctgctgtctgattgtgagagttccatgaccgagaatttgtctgacgtaccaaaaaaggagaagaaacatcgcgaaagtaaggtaaagaaagtggtttccataccggctatagacgagggagatctatggacttggcgaaagtacggtcaaaaagacatcttaggttctcgttttccaaggggttactacaggtgcgcttacaagttcacgcatggttgtaaagctacaaaacaagtccaacggagcgagaccgattcaaacatgttagctattacttacctatctgagcataaccatccacggcccactaaacgcaaggctctcgctgactccactcgttccacttcctcctccatctgctcagccataactacctctgcctcatctagagtcttccaaaacaaagacgaaccaaatcaaccccacttgccttcctcctccactcctcctagaaacgcggctgtcttgtttaaaatgacggacatggaggagtttcaggacaatatggaggtggataatgacgtcgtagatacacgtacactggcattgtttccagagtttcaacatcagccggaggaagaagacccatggtcaacattcttcgatgattataatttttacttttgattgagctcactctcatcactgcaagacaagaaaaaataaccagtgagatccaaggatttatgtacacccaaggatttagtttttgcagtgtcaatgattttcagccaataataaatcattaattt

SEQ ID NO: 5 - DNA, 9399 bp, Arabidopsis thaliana
gcatgcctcgaaaacttgccctgccacgtcttatatagctctttcaggagtgtggttttaccaattccgggcatcccaacaactccaatgatacgagttcccttgtatttatcacgatccaacttctcttccaaatcttttaaccgttgttcgtttccaaaagtctcatgctttttgtctcctgaggaagttccagcgttgctattacctaaagcacccacgacggcgttgtgacttccctccggtggtattccggtcaacgctgtcttcaccgccttcacaatttcattgactttctcactctccacactgcaatttcaaggctaatgagatcatgacctccctaagttccataaaaggttctagaagtattcatagatctgtctacaattgtaatctaatcactactgtgtctaggtttgtaactgagtcaaaaaaaataaaaaaaaatggtaccttttcttgtcaatgatgatgcccataatgttaggaatcaagttaaaagcttccttccatttctttttcctctcatcacccttagccatactcctaaatctatcaccgaactttcctttcaaatctctaacggtggatggctccagcttgtagaagattggaatcgcaacgagtgttccttcatccgtacaatctttgatcttctccagctctctcacgcaccagactgactcggtgtagttgccggagaagatagccaaaacgattttggactcctctatcctcttcagcagtacatctagaggttgacctctgtcttcatagtcgtcgataaagacgttgatgttgttcaatttcaaggccgttacgagatggctgacgaatctccggcgcaaatctgccccacggaaattgatgaacacctgatgctgcggtggcttgtcttccacagtggaaatagatgatgtctccatggcccaaaagctttttcccggtgtacttggacttttcctcactggtgtgcgttagagtagcaagaatgtattgttgactattcttttctctttttttaaatacctttttgttattttttgctgttggaaaaatatcaaatgtaaaaaaacaactgagatttgagtggttgaatgtccaatgaaaaagcatcgtcggcataaacaaaaattccggcgaaatcaaaggttagttcaaagtatcaaagcgcgaaatcatgaccaattgtgaaaaggatgaggaattcgtgtgcatcagctgcgtagaagaggtacggtactctttcgtgagccacctctctgaagctctccgtcgaaaaggcataaataatgtggtcgtagatgtagatatcgatgatctgcttttcaaggagtctcaggcaaagatcgagaaagctggggtttctgtgatggttttacccggaaactgtgatccttccgaggtatggcttgacaagttcgccaaggttctcgagtgccaaaggaacaacaaggaccaggcggtggtttcagtgttgtacggtgacagtctattacgggaccaatggcttagcgagctggatttcagaggcttatcacgaattcaccaatccaggttttcgctttattctactctctgctttttttttccttatgttgaaaattggtaaaactttttggatggctagtgtagtgttttcaatatttgatgaaatatggtgcaggaaggaatgtagtgactctatacttgtagaagagattgtgagagatgtgtacgagacgcacttttatgttggacgaattggaatctattcgaagctgctggagattgaaaacatggttaacaagcaaccgataggcatccgttgtgttggaatttggggtatgcctggcataggaaagacaacacttgctaaagcagtctttgaccaaatgtctagcgcctttgatgcttcttgttttatcgaagactatgacaaatcaattcatgagaagggtctttattgtttgctggaggaacaacttttgccgggcaatgatgcaaccattatgaaactgagctcgctcagagacagattgaacagtaagagagttcttgttgttctcgatgacgtgcgcaatgctctggttggggagtcctttctcgaggggtttgactggctaggacccggaagcctgatcatcataacctctagagataaacaagtgttttgcctttgcggaatcaatcaaatatatgaggtccagggtttaaatgagaaagaggctcgtcaacttttcttgctgtctgcgtctataaaggaggatatgggagagcagaatctccaggagttgtcagtgagagtaataaattatgctaatggaaacccgttagctatcagtgtttatggaagagagctgaaaggtaagaaaaaactctcagaaatggagactgcattcctcaaactcaagcgacgtcctccatttaagattgtcgatgcatttaaaagcacctatgacacactcagtgacaacgaaaagaacatttttttggacatagcttgtttcttccagggagaaaatgtcaactacgtgatacaactgcttgagggttgtggtttctttccacatgttgaaattgatgtccttgttgacaagtgtctggtaactatttcagaaaaccgagtttggttgcataagctgacccaggatatcggccgagaaatcataaatggagaaacagtacagatcgagaggcgcagaagactgtgggaaccttggagcatcaaatatttattagaatataatgaacacaaagcaaatggagaacctaaaacaaccttcaaacgtgctcaggtttgttatattttgatagtttcatattttttctactttatgagccaacggcctaatatatccacttccaatattttcagggctctgaagagatcgaaggcctgtttctagacacatcaaacttaagatttgatctgcagccctctgcctttaagaatatgttgaaccttagattgctcaaaatttattgttccaatcctgaagtccatcctgtaatcaatttcccaacaggctctctgcattctcttcctaatgagctaagactcctccattgggagaactatcctctgaaatctttgcctcagaattttgatccaaggcaccttgtcgaaatcaacatgccgtatagtcaacttcagaaactttggggtggaaccaaggtaagcaatctctgatatgcgtcgttcacccttatcattacttgacaaattttgttgttattcatttagcttgctttttttttgtcttctcagaacctggagatgttgaggacgatcaggctttgccattcccaccatctagttgatatcgatgatctcttaaaagctgaaaatcttgaggtaattgatctccaaggttgtacgagactgcagaatttcccagccgcaggtcgattgctacgtctacgagttgtaaatctctcaggttgcataaagattaaaagtgtcctagaaattccaccaaatattgagaaactacatctacagggaactggcatattagcattaccagtttccactgttaagccaaaccatagagagcttgtgaattttctaacagaaattccgggtctttcagaggaacttgagcgtttaacaagtctgctggaatctaactcatcttgtcaagatcttgggaagcttatttgcttggagctgaaagattgctcttgtttgcagagtctgccaaacatggctaatttagatcttaatgttcttgatctctcgggttgctcaagtcttaattctattcagggtttccctcgttttctgaaacagttatatcttggtggcactgcaataagagaagtgccacaacttcctcaaagtctagaaatcttgaatgcacatggatcttgtttgcgaagtctgccaaacatggctaatttagaatttctcaaagttcttgatctctctggttgctcagagctcgagactattcagggttttcctcggaacctaaaagagttatattttgctggcactacgttaagagaagtgccccaacttcctttaagcctagaggtcttgaatgcacatggttctgactcggagaagcttcctatgcattacaagttcaacaattttttcgatctatctcaacaagtggtcaacgattttttattgaaaacgctgacttatgtaaaacacataccaagagggtatacgcaggtaatactctctctccttctgcctctctcttgtctacttacctctccattagctctctttctttccatcaccccctcaatcctttctctttctctgttcgcctatgtctcccgctctctatagtgcctgttatgtacacccaaccacaccctcctcatatcatatctgacttatttgtattgcaacaggaactcatcaacaaagctccgactttcagcttcagtgcgccctcacatacaaatcaaaacgccacatttgatctgcaatcaggatcttctgtaatgacacgactaaatcattcatggaggaacacgcttgtgggatttggtatgctggtggaagttgcatttcccgaggactactgtgatgctacagatgttggcataagttgtgtttgcagatggagcaacaaagaaggccgctcttgtaggatagaaagaaaatttcattgttgggcaccatggcaagttgttccaaaagttcgaaaggatcatacgtttgtctttagtgatgtcaacatgcgcccaagtaccggtgaaggaaatgaccctgatatctgggctggattagttgtatttgagttctttcctatcaatcagcagacaaagtgtctaaatgataggttcacagtgagaagatgtggagtccgtgtaataaatgttgcaactggcaatacaagtcttgagaacatagcactagttttgtctttggatccagtagaggtttccggttatgaagtattgagagtcagctatgatgatttacaggagatggataaagttctatttctttacatagcgtctttgttcaatgacgaggatgttgattttgtggcaccacttattgccggtattgacttggatgttagctctgggctcaaggtcttagccgatgtgtctctcataagtgtatcatcaaatggggaaatagtgatgcatagtttgcaaagacaaatgggtaaagaaatcctccatggacaatccatgctgctgtctgattgtgagagttccatgaccgagaatttgtctgacgtaccaaaaaagtaagttctctttttctatcagcttcatatacaaccgtagacttaaataaatttgaaaatatttgagtacataaaatcacattaggtgcaatgaaaaaacgtgcaatttattagattgaactaaagtttacaaaaaagccattagagatgtaccagtatgctaccttttccaagttgcttaataacataggtttttcatgtttgtactttttagattaatttaaaaatccgttttttactccattcaagattttagaaaagtttattttttatttaatgaaatattagatttgttgtgaaaaagacattaatttacaatcagaggtaaaatctctagagacatttggcatttttgctcggctttctttgttaaatccttttttcgtgaggattttagaaaagttgattgtgagctactcaccaaggtttcatctttttgaaaaatactcttacataagatgtgtcaaggaaatgttagtttatctaacaaaaacccacttctcaacgtttccatatctatgtatctttttactttcatgaaaccatgagagacatatatgcattatgaaattttctgaattcttacattccttatagaacttctctcccacccacaaagatttgggaatcattagtcttttttattttttttcaattttcaattgatgtttgcttttgttttacttcttattatcatgttaaaaacttttttgatgccataaaaataacctttatacgtatgttgtaatcgtttttatggtaacatatattagatttttaaactttgcaaaaaaaaaaaaaaaaaaaacattagatttttaaaattctaaaagtagggttaaactccgtgtcgatagcaaaaaagggttacattacgtgttactttttactttgtgtgattaatttgcattttagaggtagagggtaaaattgcaaaaatttggcgaatgaaaatttttttttgcacaaaaccttatcttaaaccgaactgatcatggcacacactaacattgatcacagaactagctataaatcacaatatataatcacatgggtttcgatatatgttatatcgacgttggatgcaggaagaagaaacatagcgaaagtagggtaaagaaagtggtttccataccggctatagacgagggagatctatggacttggcgaaagtacggtcaaaaagacatcttaggttctcgttttccaaggtacactcatgtatttttgtatatacatattcctatatttgtgtatatattatattctttacaaacataataaatagttatacatataaacaaattatttaaatccaacaaaaacaaaatagtattttacaaaaggaaagttttgtaacaacttgcaaaacaccattttttatacacaaacgtaatcatcctaaacttgctggtggcctggtaaaccttcatatgttgctggaaaagtgaatatttctaagaaaaaacaaaaattattaaccattgaaaaagtcacattaagcagaatactgcagctttgtaaaatagaaagttcatttaattatctacggcgcaaattataagcctttaaattcttttttatgatgcagacttatcttataatcacaatattccatttgaaggatattatagatgtttttttttttttttaatccagttgtgcgtaagtgatcaaattatggttagttatgacttttgatggtcacttgatgacttaattaattatttgttcctcaggggttactacaggtgcgcttacaagttcacgcatggttgtaaagctacaaaacaagtccaacggagcgagaccgattcaaacatgttagctattacttacctatctgagcataaccatccacggcccactaaacgcaaggctctcgctgactccactcgttccacttcctcctccatctgctgagccataactacctctgcctcatctagagtcttccaaaacaaagacgaaccaaatcaaccccacttgccttcctcctccactcctcctggaaacgcggctgtcttgtttaaaatgacggacatggaggagtttcaggacaatatggaggtggataatgacgtcgtagatacacgtacactggcattgtttccagagtttcaacatcagccggaggaagaatacccatggtcaacattcttcgatgattataatttttgtttttattgagctcactctcatcactgcaagacaagaaaaaataaccagtgagatccaaggatttatgtacacccaaggatttagtttttgcagtgtcaatgattttcagccaataataaatcattaatttcaattaccagtcctccccaacctgtaaacccatcatgcctccaacatatggtgatctcgacaaattcgaaagcctaataccaaggatacatgtatcacagtaggatttctcaatcctgatgcactcgaagaggaatagatcttaagaaatcattaagccttatgaaagcaaggaaagaaatggctgagatgtcttacgttgattacaaatagaggcaatcaacacaacaattgttttgttgtgggttttgtgtcgatataccattgcggttaagagcctgccaaatgggtatgtaagctagagatctctctcatgtgtttattctctttgatttgtgtaaagctttgaactttcctattttcaataaaaccatagtctgaagaggtgaatttctttatccctattttcaactaaagatgtagtatatgaaaattacctaaaatctttttcaaaatcctttcttaaaaggtttgataatttttgaataacaatggatttgatttgagtttcacaaatcattgattgaatagaagaaaaggatggtaaatgattttgaattattttacataaatcaataattaataacaggtgattaataaaaaagactctaaatcttcaaagagcatctccataagagtatcttaacaataaaatgatattatttctatagaatttagttgtttaattaaatttaaatttgttacaaaattaacccaacaatcaagtgacatgggtaaaatagagtttctaaatcataagtttctcaaaagttacatgtgtaatataccggctatgctttggtttctgttgtctgtgaagatgatccttctggaaggtctcgaaggcttcctctggactccccttcgattcatgtttctgtattgacgcgatgtctagatcttccgtttgtgacatagtgaatgaattaagaaggggtttgtttctttcagtagtttaaggatgcgttttgtaacctcggaattagtttggactcggccacttgtgtatttctaagttcttagtatttgatctttggcttgctttgtggttacctttccttcaagctttaggattttagtagttgagactcttgcagaagtttaggattgattggattgcactcatggctccgctgggtcgcgcgttaagtgtaaaggttcctaggacgggggttaagtgagagatttgatttgtcctctcttagtattttattttctgcttgaactttctgatatatctcctctatgtctttgatccttcttagtttgatccatttcttttgaccttgctaagatggtttcaaggcttaatttcatattttggcaaaaaacagatggaatgcagtgttatagtgggttgaaaccggagatattgagttaatctctcttccaccggttgttgaaatattttcaagcctatggtaggttttcatgtgcccctttctcgtaaaatcaacaagctgaagcactattggtatctcatgtagtgtttgaactactctcttatgtggtttaagattattaagaatttcacaatgcaaatcacagatatgtaatcgagtagctatgtgtttggctaaacaagagtttcgcattccaatgtatggttcctttgattgatatataaaagttacactttgtgaaaaaaaaaaaaaaaaaaaaacaggaatccttcacttttttctattacttttgaaattttttattgtgattttagataccctaatgaagatgctataaagcaaatccctctttctttttatttctgctaaacaatatcaaaaattctgtgcacgaacacatctctgcacaagtttgggtcgccattttcaattatcccatattcattggaggaaatactaagaatagagcatgc

SEQ ID NO: 6 - DNA, 1183 bp, Arabidopsis thaliana
gcatgcctcgaaaacttgccctgccacgtcttatatagctctttcaggagtgtggttttaccaattccgggcatcccaacaactccaatgatacgagttcccttgtatttatcacgatccaacttctcttccaaatcttttaaccgttgttcgtttccaaaagtctcatgctttttgtctcctgaggaagttccagcgttgctattacctaaagcacccacgacggcgttgtgacttccctccggtggtattccggtcaacgctgtcttcaccgccttcacaatttcattgactttctcactctccacactgcaatttcaaggctaatgagatcatgacctccctaagttccataaaaggttctagaagtattcatagatctgtctacaattgtaatctaatcactactgtgtctaggtttgtaactgagtcaaaaaaaataaaaaaaaatggtaccttttcttgtcaatgatgatgcccataatgttaggaatcaagttaaaagcttccttccatttctttttcctctcatcacccttagccatactcctaaatctatcaccgaactttcctttcaaatctctaacggtggatggctccagcttgtagaagattggaatcgcaacgagtgttccttcatccgtacaatctttgatcttctccagctctctcacgcaccagactgactcggtgtagttgccggagaagatagccaaaacgattttggactcctctatcctcttcagcagtacatctagaggttgacctctgtcttcatagtcgtcgataaagacgttgatgttgttcaatttcaaggccgttacgagatggctgacgaatctccggcgcaaatctgccccacggaaattgatgaacacctgatgctgcggtggcttgtcttccacagtggaaatagatgatgtctccatggcccaaaagctttttcccggtgtacttggacttttcctcactggtgtgcgttagagtagcaagaatgtattgttgactattcttttctctttttttaaatacctttttgttattttttgctgttggaaaaatatcaaatgtaaaaaaacaactgagatttgagtggttgaatgtccaatgaaaaagcatcgtcggcataaacaaaaattccggcgaaatcaaaggttagttcaaagtatcaaagcgcgaaatc

SEQ ID NO: 7 - DNA, 2263 bp, Arabidopsis thaliana
gccataactacctctgcctcatctagagtcttccaaaacaaagacgaaccaaatcaaccccacttgccttcctcctccactcctcctggaaacgcggctgtcttgtttaaaatgacggacatggaggagtttcaggacaatatggaggtggataatgacgtcgtagatacacgtacactggcattgtttccagagtttcaacatcagccggaggaagaatacccatggtcaacattcttcgatgattataatttttgtttttattgagctcactctcatcactgcaagacaagaaaaaataaccagtgagatccaaggatttatgtacacccaaggatttagtttttgcagtgtcaatgattttcagccaataataaatcattaatttcaattaccagtcctccccaacctgtaaacccatcatgcctccaacatatggtgatctcgacaaattcgaaagcctaataccaaggatacatgtatcacagtaggatttctcaatcctgatgcactcgaagaggaatagatcttaagaaatcattaagccttatgaaagcaaggaaagaaatggctgagatgtcttacgttgattacaaatagaggcaatcaacacaacaattgttttgttgtgggttttgtgtcgatataccattgcggttaagagcctgccaaatgggtatgtaagctagagatctctctcatgtgtttattctctttgatttgtgtaaagctttgaactttcctattttcaataaaaccatagtctgaagaggtgaatttctttatccctattttcaactaaagatgtagtatatgaaaattacctaaaatctttttcaaaatcctttcttaaaaggtttgataatttttgaataacaatggatttgatttgagtttcacaaatcattgattgaatagaagaaaaggatggtaaatgattttgaattattttacataaatcaataattaataacaggtgattaataaaaaagactctaaatcttcaaagagcatctccataagagtatcttaacaataaaatgatattatttctatagaatttagttgtttaattaaatttaaatttgttacaaaattaacccaacaatcaagtgacatgggtaaaatagagtttctaaatcataagtttctcaaaagttacatgtgtaatataccggctatgctttggtttctgttgtctgtgaagatgatccttctggaaggtctcgaaggcttcctctggactccccttcgattcatgtttctgtattgacgcgatgtctagatcttccgtttgtgacatagtgaatgaattaagaaggggtttgtttctttcagtagtttaaggatgcgttttgtaacctcggaattagtttggactcggccacttgtgtatttctaagttcttagtatttgatctttggcttgctttgtggttacctttccttcaagctttaggattttagtagttgagactcttgcagaagtttaggattgattggattgcactcatggctccgctgggtcgcgcgttaagtgtaaaggttcctaggacgggggttaagtgagagatttgatttgtcctctcttagtattttattttctgcttgaactttctgatatatctcctctatgtctttgatccttcttagtttgatccatttcttttgaccttgctaagatggtttcaaggcttaatttcatattttggcaaaaaacagatggaatgcagtgttatagtgggttgaaaccggagatattgagttaatctctcttccaccggttgttgaaatattttcaagcctatggtaggttttcatgtgcccctttctcgtaaaatcaacaagctgaagcactattggtatctcatgtagtgtttgaactactctcttatgtggtttaagattattaagaatttcacaatgcaaatcacagatatgtaatcgagtagctatgtgtttggctaaacaagagtttcgcattccaatgtatggttcctttgattgatatataaaagttacactttgtgaaaaaaaaaaaaaaaaaaaaacaggaatccttcacttttttctattacttttgaaattttttattgtgattttagataccctaatgaagatgctataaagcaaatccctctttctttttatttctgctaaacaatatcaaaaattctgtgcacgaacacatctctgcacaagtttgggtcgccattttcaattatcccatattcattggaggaaatactaagaatagagcatgc

SEQ ID NO: 8 - DNA, 4336 bp, Arabidopsis thaliana
gtccaatgaaaaagcatcgtcggcataaacaaaaattccggcgaaatcaaaggttagttcaaagtatcaaagcgcgaaatcatgaccaattgtgaaaaggatgaggaattcgtgtgcatcagctgcgtagaagaggtacggtactctttcgtgagccacctctctgaagctctccgtcgaaaaggcataaataatgtggtcgtagatgtagatatcgatgatctgcttttcaaggagtctcaggcaaagatcgagaaagctggggtttctgtgatggttttacccggaaactgtgatccttccgaggtatggcttgacaagttcgccaaggttctcgagtgccaaaggaacaacaaggaccaggcggtggtttcagtgttgtacggtgacagtctattacgggaccaatggcttagcgagctggatttcagaggcttatcacgaattcaccaatccaggaaggaatgtagtgactctatacttgtagaagagattgtgagagatgtgtacgagacgcacttttatgttggacgaattggaatctattcgaagctgctggagattgaaaacatggttaacaagcaaccgataggcatccgttgtgttggaatttggggtatgcctggcataggaaagacaacacttgctaaagcagtctttgaccaaatgtctagcgcctttgatgcttcttgttttatcgaagactatgacaaatcaattcatgagaagggtctttattgtttgctggaggaacaacttttgccgggcaatgatgcaaccattatgaaactgagctcgctcagagacagattgaacagtaagagagttcttgttgttctcgatgacgtgcgcaatgctctggttggggagtcctttctcgaggggtttgactggctaggacccggaagcctgatcatcataacctctagagataaacaagtgttttgcctttgcggaatcaatcaaatatatgaggtccagggtttaaatgagaaagaggctcgtcaacttttcttgctgtctgcgtctataaaggaggatatgggagagcagaatctccaggagttgtcagtgagagtaataaattatgctaatggaaacccgttagctatcagtgtttatggaagagagctgaaaggtaagaaaaaactctcagaaatggagactgcattcctcaaactcaagcgacgtcctccatttaagattgtcgatgcatttaaaagcacctatgacacactcagtgacaacgaaaagaacatttttttggacatagcttgtttcttccagggagaaaatgtcaactacgtgatacaactgcttgagggttgtggtttctttccacatgttgaaattgatgtccttgttgacaagtgtctggtaactatttcagaaaaccgagtttggttgcataagctgacccaggatatcggccgagaaatcataaatggagaaacagtacagatcgagaggcgcagaagactgtgggaaccttggagcatcaaatatttattagaatataatgaacacaaagcaaatggagaacctaaaacaaccttcaaacgtgctcagggctctgaagagatcgaaggcctgtttctagacacatcaaacttaagatttgatctgcagccctctgcctttaagaatatgttgaaccttagattgctcaaaatttattgttccaatcctgaagtccatcctgtaatcaatttcccaacaggctctctgcattctcttcctaatgagctaagactcctccattgggagaactatcctctgaaatctttgcctcagaattttgatccaaggcaccttgtcgaaatcaacatgccgtatagtcaacttcagaaactttggggtggaaccaagaacctggagatgttgaggacgatcaggctttgccattcccaccatctagttgatatcgatgatctcttaaaagctgaaaatcttgaggtaattgatctccaaggttgtacgagactgcagaatttcccagccgcaggtcgattgctacgtctacgagttgtaaatctctcaggttgcataaagattaaaagtgtcctagaaattccaccaaatattgagaaactacatctacagggaactggcatattagcattaccagtttccactgttaagccaaaccatagagagcttgtgaattttctaacagaaattccgggtctttcagaggaacttgagcgtttaacaagtctgctggaatctaactcatcttgtcaagatcttgggaagcttatttgcttggagctgaaagattgctcttgtttgcagagtctgccaaacatggctaatttagatcttaatgttcttgatctctcgggttgctcaagtcttaattctattcagggtttccctcgttttctgaaacagttatatcttggtggcactgcaataagagaagtgccacaacttcctcaaagtctagaaatcttgaatgcacatggatcttgtttgcgaagtctgccaaacatggctaatttagaatttctcaaagttcttgatctctctggttgctcagagctcgagactattcagggttttcctcggaacctaaaagagttatattttgctggcactacgttaagagaagtgccccaacttcctttaagcctagaggtcttgaatgcacatggttctgactcggagaagcttcctatgcattacaagttcaacaattttttcgatctatctcaacaagtggtcaacgattttttattgaaaacgctgacttatgtaaaacacataccaagagggtatacgcaggaactcatcaacaaagctccgactttcagcttcagtgcgccctcacatacaaatcaaaacgccacatttgatctgcaatcaggatcttctgtaatgacacgactaaatcattcatggaggaacacgcttgtgggatttggtatgctggtggaagttgcatttcccgaggactactgtgatgctacagatgttggcataagttgtgtttgcagatggagcaacaaagaaggccgctcttgtaggatagaaagaaaatttcattgttgggcaccatggcaagttgttccaaaagttcgaaaggatcatacgtttgtctttagtgatgtcaacatgcgcccaagtaccggtgaaggaaatgaccctgatatctgggctggattagttgtatttgagttctttcctatcaatcagcagacaaagtgtctaaatgataggttcacagtgagaagatgtggagtccgtgtaataaatgttgcaactggcaatacaagtcttgagaacatagcactagttttgtctttggatccagtagaggtttccggttatgaagtattgagagtcagctatgatgatttacaggagatggataaagttctatttctttacatagcgtctttgttcaatgacgaggatgttgattttgtggcaccacttattgccggtattgacttggatgttagctctgggctcaaggtcttagccgatgtgtctctcataagtgtatcatcaaatggggaaatagtgatgcatagtttgcaaagacaaatgggtaaagaaatcctccatggacaatccatgctgctgtctgattgtgagagttccatgaccgagaatttgtctgacgtaccaaaaaagaagaagaaacatagcgaaagtagggtaaagaaagtggtttccataccggctatagacgagggagatctatggacttggcgaaagtacggtcaaaaagacatcttaggttctcgttttccaaggggttactacaggtgcgcttacaagttcacgcatggttgtaaagctacaaaacaagtccaacggagcgagaccgattcaaacatgttagctattacttacctatctgagcataaccatccacggcccactaaacgcaaggctctcgctgactccactcgttccacttcctcctccatctgctgagccataactacctctgcctcatctagagtcttccaaaacaaagacgaaccaaatcaaccccacttgccttcctcctccactcctcctggaaacgcggctgtcttgtttaaaatgacggacatggaggagtttcaggacaatatggaggtggataatgacgtcgtagatacacgtacactggcattgtttccagagtttcaacatcagccggaggaagaatacccatggtcaacattcttcgatgattataatttttgtttttattgagctcactctcatcactgcaagacaagaaaaaataaccagtgagatccaaggatttatgtacacccaaggatttagtttttgcagtgtcaatgattttcagccaataataaatcattaattt

SEQ ID NO: 9 - protein, 1378 aa, Arabidopsis thaliana
MTNCEKDEEFVCISCVEEVRYSFVSHLSEALRRKGINNVVVDVDIDDLLFKESQAKIEKAGVSVMVLPGNCDPSEVWLDKFAKVLECQRNNKDQAVVSVLYGDSLLRDQWLSELDFRGLSRIHQSRKECSDSILVEEIVRDVYETHFYVGRIGIYSKLLEIENMVNKQPIGIRCVGIWGMPGIGKTTLAKAVFDQMSSAFDASCFIEDYDKSIHEKGLYCLLEEQLLPGNDATIMKLSSLRDRLNSKRVLVVLDDVCNALVAESFLEGFDWLGPGSLIIITSRDKQVFRLCGINQIYEVQGLNEKEARQLFLLSASIMEDMGEQNLHELSVRVISYANGNPLAISVYGRELKGKKKLSEMETAFLKLKRRPPFKIVDAFKSSYDTLSDNEKNIFLDIACFFQGENVNYVIQLLEGCGFFPHVEIDVLVDKCLVTISENRVWLHKLTQDIGREIINGETVQIERRRRLWEPWSIKYLLEYNEHKANGEPKTTFKRAQGSEEIEGLFLDTSNLRFDLQPSAFKNMLNLRLLKIYCSNPEVHPVINFPTGSLHSLPNELRLLHWENYPLKSLPQNFDPRHLVEINMPYSQLQKLWGGTKNLEMLRTIRLCHSQHLVDIDDLLKAENLEVIDLQGCTRLQNFPAAGRLLRLRVVNLSGCIKIKSVLEIPPNIEKLHLQGTGILALPVSTVKPNHRELVNFLTEIPGLSEASKLERLTSLLESNSSCQDLGKLICLELKDCSCLQSLPNMANLDLNVLDLSGCSSLNSIQGFPRFLKQLYLGGTAIREVPQLPQSLEILNAHGSCLRSLPNMANLEFLKVLDLSGCSELETIQGFPRNLKELYFAGTTLREVPQLPLSLEVLNAHGSDSEKLPMHYKFNNFFDLSQQVVNDFFLKALTYVKHIPRGYTQELINKAPTFSFSAPSHTNQNATFDLQPGSSVMTRLNHSWRNTLVGFGMLVEVAFPEDYCDATDVGISCVCRWSNKEGRSCRIERNFHCWAPGKVVPKVRKDHTFVFSDVNMRPSTGEGNDPDIWAGLVVFEFFPINQQTKCLNDRFTVTRCGVRVINVATGNTSLENISLVLSLDPVEVSGYEVLRVSYDDLQEMDKVLFLYIASLFNDEDVDFVAPLIAGIDLDVSSGLKVLADVSLISVSSNGEIVMHSLQRQMGKEILHGQSMLLSDCESSMTENLSDVPKKEKKHRESKVKKVVSIPAIDEGDLWTWRKYGQKDILGSRFPRGYYRCAYKFTHGCKATKQVQRSETDSNMLAITYLSEHNHPRPTKRKALADSTRSTSSSICSAITTSASSRVFQNKDEPNQPHLPSSSTPPRNAAVLFKMTDMEEFQDNMEVDNDVVDTRTLALFPEFQHQPEEEDPWSTFFDDYNFYF

SEQ ID NO: 10 - protein, 1288 aa, Arabidopsis thaliana
MTNCEKDEEFVCISCVEEVRYSFVSHLSEALRRKGINNVVVDVDIDDLLFKESQAKIEKAGVSVMVLPGNCDPSEVWLDKFAKVLECQRNNKDQAVVSVLYGDSLLRDQWLSELDFRGLSRIHQSRKECSDSILVEEIVRDVYETHFYVGRIGIYSKLLEIENMVNKQPIGIRCVGIWGMPGIGKTTLAKAVFDQMSSAFDASCFIEDYDKSIHEKGLYCLLEEQLLPGNDATIMKLSSLRDRLNSKRVLVVLDDVRNALVGESFLEGFDWLGPGSLIIITSRDKQVFCLCGINQIYEVQGLNEKEARQLFLLSASIKEDMGEQNLQELSVRVINYANGNPLAISVYGRELKGKKKLSEMETAFLKLKRRPPFKIVDAFKSTYDTLSDNEKNIFLDIACFFQGENVNYVIQLLEGCGFFPHVEIDVLVDKCLVTISENRVWLHKLTQDIGREIINGETVQIERRRRLWEPWSIKYLLEYNEHKANGEPKTTFKRAQGSEEIEGLFLDTSNLRFDLQPSAFKNMLNLRLLKIYCSNPEVHPVINFPTGSLHSLPNELRLLHWENYPLKSLPQNFDPRHLVEINMPYSQLQKLWGGTKNLEMLRTIRLCHSHHLVDIDDLLKAENLEVIDLQGCTRLQNFPAAGRLLRLRVVNLSGCIKIKSVLEIPPNIEKLHLQGTGILALPVSTVKPNHRELVNFLTEIPGLSEELERLTSLLESNSSCQDLGKLICLELKDCSCLQSLPNMANLDLNVLDLSGCSSLNSIQGFPRFLKQLYLGGTAIREVPQLPQSLEILNAHGSCLRSLPNMANLEFLKVLDLSGCSELETIQGFPRNLKELYFAGTTLREVPQLPLSLEVLNAHGSDSEKLPMHYKFNNFFDLSQQVVNDFLLKTLTYVKHIPRGYTQELINKAPTFSFSAPSHTNQNATFDLQSGSSVMTRLNHSWRNTLVGFGMLVEVAFPEDYCDATDVGISCVCRWSNKEGRSCRIERKFHCWAPWQVVPKVRKDHTFVFSDVNMRPSTGEGNDPDIWAGLVVFEFFPINQQTKCLNDRFTVRRCGVRVINVATGNTSLENIALVLSLDPVEVSGYEVLRVSYDDLQEMDKVLFLYIASLFNDEDVDFVAPLIAGIDLDVSSGLKVLADVSLISVSSNGEIVMHSLQRQMGKEILHGQSMLLSDCESSMTENLSDVPKKKKKHSESRVKKVVSIPAIDEGDLWTWRKYGQKDILGSRFPRGYYRCAYKFTHGCKATKQVQRSETDSNMLAITYLSEHNHPRPTKRKALADSTRSTSSSIC

SEQ ID NO: 11 - DNA, 18 bp, Homo sapiens
agctcgagactattcagg

SEQ ID NO: 12 - DNA, 19 bp, Homo sapiens
aaacactgatagctaacgg

SEQ ID NO: 13 - DNA, 18 bp, Homo sapiens
atctctaacggtggatgg

SEQ ID NO: 14 - DNA, 19 bp, Homo sapiens
tgcattcaagacctctagg

SEQ ID NO: 15 - DNA, 19 bp, Homo sapiens
ccgttagctatcagtgttt

SEQ ID NO: 16 - DNA, 18 bp, Homo sapiens
agtcatcaagtgaccatc

SEQ ID NO: 17 - DNA, 18 bp, Homo sapiens
ctagaggtcttgaatgca

SEQ ID NO: 18 - DNA, 18 bp, Homo sapiens
gcatcacagtagtcctcg

SEQ ID NO: 19 - DNA, 19 bp, Homo sapiens
acatccaagtcaataccgg

SEQ ID NO: 20 - DNA, 18 bp, Homo sapiens
gccaatagagatgtacca

SEQ ID NO: 21 - DNA, 18 bp, Homo sapiens
tggtacatctctaatggc

SEQ ID NO: 22 - DNA, 19 bp, Homo sapiens
agtaacacgtaatgtaacc

SEQ ID NO: 23 - DNA, 19 bp, Homo sapiens
accagcaagtttaggatga

SEQ ID NO: 24 - DNA, 18 bp, Homo sapiens
gatggtcacttgatgact

SEQ ID NO: 25 - DNA, 19 bp, Homo sapiens
ggtgtacataaatccttgg

SEQ ID NO: 26 - DNA, 19 bp, Homo sapiens
ccaaggatttatgtacacc

SEQ ID NO: 27 - DNA, 18 bp, Homo sapiens
actcttatggagatgctc

SEQ ID NO: 28 - DNA, 18 bp, Homo sapiens
cgcatccttaaactactg

SEQ ID NO: 29 - DNA, 18 bp, Homo sapiens
atatctccggtttcaacc

SEQ ID NO: 30 - DNA, 18 bp, Homo sapiens
ccttggtgagtagctcac

SEQ ID NO: 31 - DNA, 18 bp, Homo sapiens
ccatagatctccctcgtc

SEQ ID NO: 32 - DNA, 19 bp, Homo sapiens
ccttatagaacttctctcc

SEQ ID NO: 33 - DNA, 18 bp, Homo sapiens
ctcttcgagtgcatcagg

SEQ ID NO: 34 - DNA, 19 bp, Homo sapiens
ccggtattgacttggatgt

SEQ ID NO: 35 - DNA, 18 bp, Homo sapiens
agatacacgtacactggc

SEQ ID NO: 36 - DNA, 18 bp, Homo sapiens
tccagcccagatatcagg

SEQ ID NO: 37 - DNA, 18 bp, Homo sapiens
tgcataggaagcttctcc

SEQ ID NO: 38 - DNA, 18 bp, Homo sapiens
ttcagaggaacttgagcg

SEQ ID NO: 39 - DNA, 19 bp, Homo sapiens
ccaagcaaataagcttccc

SEQ ID NO: 40 - DNA, 18 bp, Homo sapiens
atcgtcctcaacatctcc

SEQ ID NO: 41 - DNA, 18 bp, Homo sapiens
aggcgcagaagactgtgg

SEQ ID NO: 42 - DNA, 18 bp, Homo sapiens
ttgatgctccaaggttcc

SEQ ID NO: 43 - DNA, 18 bp, Homo sapiens
gagatgtgtacgagacgc

SEQ ID NO: 44 - DNA, 18 bp, Homo sapiens
caatctccagcagcttcg

SEQ ID NO: 45 - DNA, 18 bp, Homo sapiens
ttgagtggttgaatgtcc

SEQ ID NO: 46 - DNA, 18 bp, Homo sapiens
cacacgaattcctcatcc

SEQ ID NO: 47 - DNA, 18 bp, Homo sapiens
tgaaggaacactcgttgc

SEQ ID NO: 48 - DNA, 18 bp, Homo sapiens
gtctttcagaggcctcga

SEQ ID NO: 49 - DNA, 18 bp, Homo sapiens
ggtaagcaatctctgata

SEQ ID NO: 50 - DNA, 18 bp, Homo sapiens
atgttatatcgacgttgg

SEQ ID NO: 51 - DNA, 18 bp, Homo sapiens
gaggaagtggaacgagtg

SEQ ID NO: 52 - DNA, 18 bp, Homo sapiens
aactcctccatgtccgtc

SEQ ID NO: 53 - DNA, 26 bp, Homo sapiens
atctccctcgtctatagccggtatgg

SEQ ID NO: 54 - DNA, 27 bp, Homo sapiens
gatcaggcttccgggtcctagccagtc

SEQ ID NO: 55 - DNA, 27 bp, Homo sapiens
agtgatgtcaacatgcgcccaagtacc

SEQ ID NO: 56 - DNA, 26 bp, Homo sapiens
aaccttcaaacgtgctcagggctctg

SEQ ID NO: 57 - DNA, 26 bp, Homo sapiens
acatctccaggttcttggttccaccc

SEQ ID NO: 58 - DNA, 29 bp, Homo sapiens
gtcgacatgaccaattgtgaaaaggatga

SEQ ID NO: 59 - DNA, 27 bp, Homo sapiens
gtcgaccttgtcttgcagtgatgagag

SEQ ID NO: 60 - DNA, 23 bp, Homo sapiens
ctattccatggaggaggaagtgg

SEQ ID NO: 61 - DNA, 24 bp, Homo sapiens
ttagtcgacgaagaagaaacatag

(19) United States
(12) Patent Application Publication
(10) Pub. No.: US 2003/0196215 A1
(43) Pub. Date: Oct. 16, 2003

(54) Novel class of proteins and uses thereof for plant resistance to various pathogenic agents

(75) Inventors: Jocelyne Olivier, Toulouse (FR); Laurent Deslandes, Toulouse (FR); Yves Marco, Castanet Tolosan (FR)

Correspondence Address: Mark B. Wilson, Fulbright & Jaworski L.L.P., Suite 2400, 600 Congress Avenue, Austin, TX, 78701, US

(73) Assignees: Institut National de le Recherche Agronomique (INRA), Paris Cedex (FR); Centre National de la Recherche Scientifique (CNRS), Paris Cedex (FR)

(21) Appl. No.: 10/114,824

(22) Filed: Apr. 1, 2002

(51) Int. Cl. A01H 1/00; C12N 15/82; C07H 21/04

(52) U.S. Cl. 800/279; 536/23.6

(57) ABSTRACT

Disclosed is substantially pure DNA encoding an Arabidopsis thaliana Rps2 polypeptide; substantially pure Rps2 polypeptide; and methods of using such DNA to express the Rps2 polypeptide in plant cells and whole plants to provide, in transgenic plants, disease resistance to pathogens. Also disclosed are conserved regions characteristic of the RPS family and primers and probes for the identification and isolation of additional RPS disease-resistance genes.

This invention relates to a new class of proteins having an N-terminal portion containing characteristic units of a plant protein of resistance to pathogenic agents and a C-terminal portion containing a DNA-binding domain.

The invention also relates to nucleic acids coding for such proteins involved in plant resistance to various pathogens and also to means for detecting these proteins and these nucleic acids, such as antibodies or nucleotide probes and primers.

The invention also relates to recombinant vectors containing a nucleic acid coding for a polypeptide belonging to this new class of proteins, to host cells transformed by a nucleic acid or a recombinant vector according to the invention, and to transgenic plants of which some or all of the cells are transformed by a nucleic acid or a recombinant vector according to the invention.

The invention also relates to means for increasing or, conversely, inhibiting the expression of a nucleic acid coding for a protein according to the invention in plants, with the aim of increasing the resistance of said plants to various pathogens.

The invention further relates to methods for screening candidate substances fixing to a polypeptide according to the invention, and to candidate substances obtained according to such methods.

The improvement of the resistance of plants, and particularly of plants of agronomic interest, to different pathogens is the subject of much research, with the aim of satisfying the technical needs of an agricultural industry sector which more and more uses intensive culture methods.

The identification of new genes or new proteins able to provide to plants an increased resistance to different bacteria, fungi or viruses has assumed a major economic importance in today's agriculture, enabling the development of large-scale crops with natural resistance to these pathogens and thus not requiring substantial applications of agrochemical products, in particular pesticides, which have several adverse effects on the environment.

Bacterial wilting is one of the most widespread diseases affecting agricultural crops. It is one of the most important phytobacterioses in the world and is mainly caused by the bacterium Ralstonia solanacearum.

This vascular pathogen, of soil origin, affects more than 200 plant species including tomato, tobacco, potato or banana, mainly in tropical or sub-tropical zones.

Various strains of this pathogen have recently been detected in Europe. Study of the resistance of the tomato plant to Ralstonia solanacearum is complicated by its polygenic character. Several genes involved in the establishment of the resistance have been discovered.

A study by DESLANDES et al. (1998) has recently identified, in the plant Arabidopsis thaliana, a chromosome locus carrying a genetic determinant involved in the resistance to Ralstonia solanacearum. Thus lines of Arabidopsis thaliana, comprising the F₉ generation, obtained by crossing lines of Arabidopsis thaliana respectively sensitive to and resistant to Ralstonia solanacearum, were used to study the co-segregation of markers of the RLFP type with the expression of the resistance to the phenotype of this pathogen responsible for wilting.

These authors also showed that a genetic determinant of the resistance to Ralstonia solanacearum was localized on chromosome V of Arabidopsis thaliana, between the RFLP markers mi83 and mi61, in other words in a region of about 6.8 cM (approximately 1.7 to 3.4 Mb) of this chromosome.

According to these authors, the results of the study suggest that the locus of interest localized on chromosome V carries one or more genes, these genes being able either to provide the resistance phenotype, or on the other hand the phenotype of sensitivity to Ralstonia solanacearum, although the genetic basis of the resistance and/or the sensitivity to this pathogen were not identified in Arabidopsis thaliana.

In addition, DESLANDES et al. (1998) noted that the chromosome region of interest in Arabidopsis thaliana was known to contain several other genetic determinants involved in the recognition of pathogens.

The applicant has now identified a genetic determinant which is able to provide resistance to Ralstonia solanacearum in Arabidopsis thaliana.

This is a single gene coding for a polypeptide belonging to a new structural class of proteins. This new class of proteins identified according to the invention is characterized in that it has an N-terminal portion with structural units in common with several known resistance genes and a C-terminal portion having structural units in common with transcription factors containing a DNA-binding region, particularly those designated by the name of WRKY proteins or WRKY transcription factors.

It has also been shown according to the invention that the transformation of an Arabidopsis thaliana plant sensitive to Ralstonia solanacearum with a nucleic acid coding for a protein such as that defined above was able to provide resistance to different strains of this pathogen to the transformed plant.

The object of the invention is thus a nucleic acid containing at least 15 consecutive nucleotides of a nucleotide sequence coding for a protein of resistance of a plant to a pathogen, said protein comprising:

a) an N-terminal portion containing at least one amino acid sequence rich in leucine and at least one nucleotide-binding site; and

b) a C-terminal portion containing a DNA-binding domain, said binding domain comprising the amino acid sequence “WRKYGQK”.

The N-terminal portion of this protein preferably also contains at least one domain TIR(TOLL/IL-1R), such a TIR domain being principally defined by its sequence homology with the cytoplasmic domains of the TOLL protein of Drosophila and with the mammalian interleukin-1 receptor protein, whose consensus sequence is described by HAMMOND-KOSACK et al. (1997).

The N-terminal portion of this protein preferably also contains a loop P(P-LOOP), which is a peptide segment forming a loop and able to fix a phosphate, which is found in protein kinases.

Such a TIR domain preferably has the amino acid sequence

“DEEFVCISCVEEVRYSFVSHLSEALRRKGINNVVVDVDIDDLLFKESQ
AKIEKAGVSVMVLPGNCDPSEVWLDKFAKVLECQRNNKIDQAVVSVL
YGDSLLRDQWLSELDFRGLSRIHQSRKECSDSILVEEIVRDVYET”

Such a P loop preferably has the amino acid sequence “CVGIWGMPGIGKTTLAKAV”.

The N-terminal portion preferably also contains a domain of the NB-ARC type, which is a conserved domain and characteristic of pathogen resistance proteins and of proteins involved in apoptosis mechanisms.

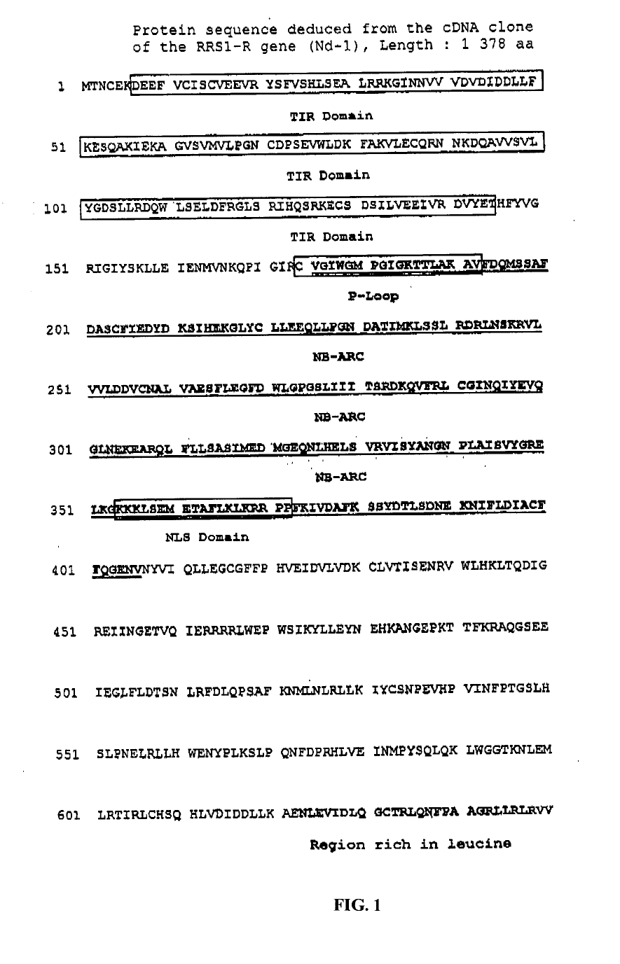

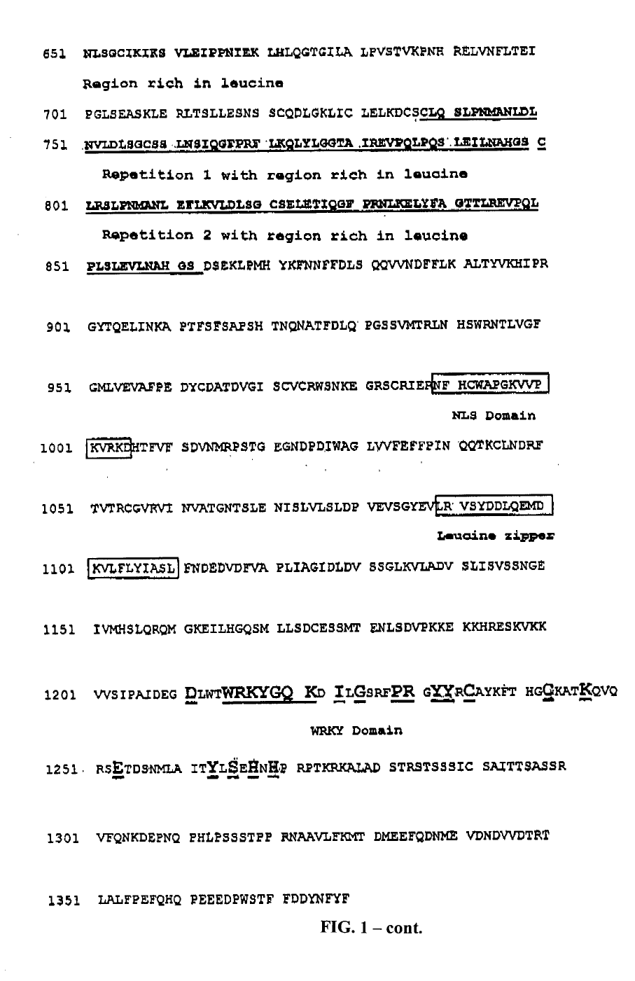

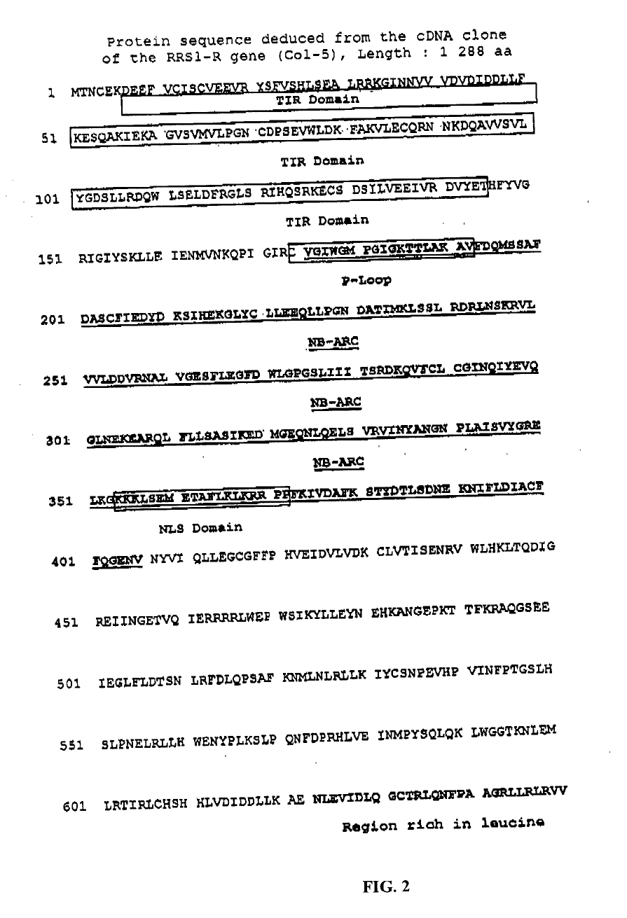

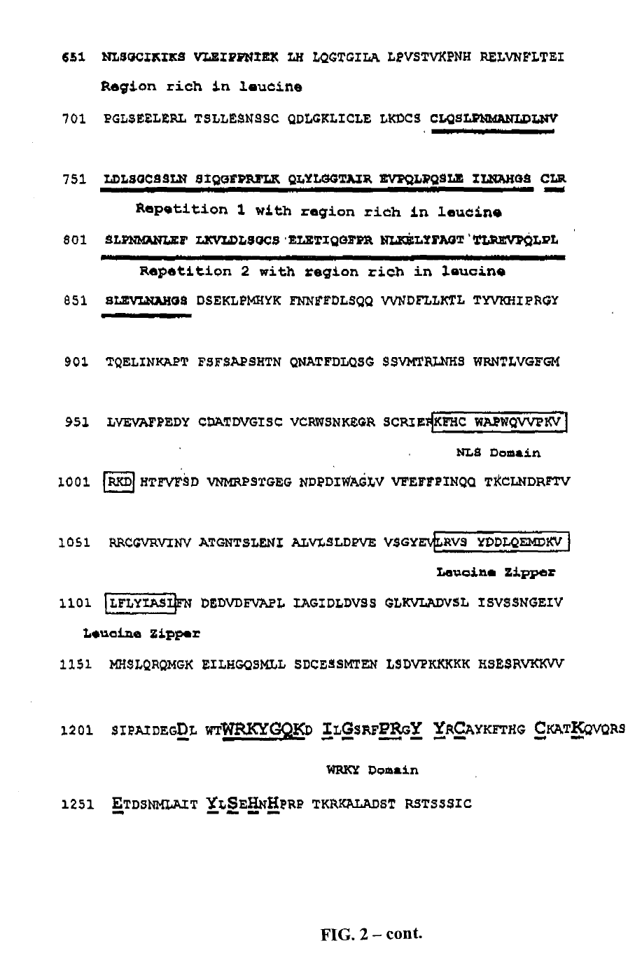

This NB-ARC domain preferably has the amino acid sequence represented in bold and underlined in FIGS. 1 and 2.

In addition, the N-terminal portion preferably contains a NLS nuclear signal site.

Such a NLS domain preferably has the amino acid sequence “KKKLSEMETAFLKLKRRPP”.

As already stated, the C-terminal portion of a protein coded by a nucleic acid according to the invention contains a WRKY domain characteristic of some protein transcription factors and characterized in that it contains the amino acid sequence “WRKYGQK”.

This WRKY domain preferably contains the following amino acid sequence: “DXXXWRKYGQKXIXGXXXPRXYYXCXXXXXXXCXXXKXXXXXEXXXXXXXXXYXSXHXH”,

in which X represents any of the 20 natural amino acids.

In addition, the C-terminal portion preferably contains a domain rich in leucine.

The C-terminal portion also preferably contains a characteristic domain of a nuclear localization signal site (NLS), said domain preferably having the amino acid sequence “NFHCWAPGKVVPKVRKD”.

The C-terminal portion also preferably contains a unit of the “leucine zipper” type, characteristic of a protein binding site, which preferably has the amino acid sequence “LRVSYDDLQEMDKVLFLYIASL”.

It is highly preferable that a nucleic acid according to the invention codes for a polypeptide containing, from its N-terminal end to its C-terminal end, (i) a TIR domain,

(ii) a P-LOOP unit,

(iii) an NB-ARC unit,

(iv) an NLS domain,

(v) a first region rich in leucine,

(vi) a second region rich in leucine,

(vii) an NLS domain,

(viii) a unit of the leucine zipper type,

(ix) a WRKY domain,

the domains (i) to (ix) being such as defined above.

A nucleic acid with complementary sequence is also in the scope of the invention.

A nucleic acid according to the invention is preferably in an isolated and/or purified form.

The term “isolated” in the context of the present invention means a biological material (nucleic acid or protein) which has been extracted from its original environment (the environment in which it is found naturally).

For example, a polynucleotide present in the natural state in a plant or an animal is not isolated. The same polynucleotide separated from the adjacent nucleic acids within which it is naturally inserted in the genome of the plant or animal is considered as “isolated”.

Such a polynucleotide may be included in a vector and/or such a polynucleotide may be included in a composition and nevertheless remain in the isolated state since the vector or the composition does not represent its natural environment.

The term “purified” does not require that the material is present in a form of absolute purity, excluding the presence of other compounds. It is more of a relative definition. A polynucleotide is in a “purified” state after purification of the starting material or the natural material by at least one order of magnitude, preferably 2 or 3 and more preferably 4 or 5 orders of magnitude.

For the purposes of the present invention, the expression “nucleotide sequence” may be used to mean either a polynucleotide or a nucleic acid. The expression “nucleotide sequence” includes the genetic material itself and is thus not limited to the information as to its sequence.

The terms “nucleic acid”, “polynucleotide”, “oligonucleotide” or “nucleotide sequence” include sequences of RNA, DNA, cDNA or RNA/DNA hybrid sequences of more than one nucleotide, either in the single or double stranded form.

The term “nucleotide” means both the natural nucleotides (A, T, G, C) and modified nucleotides which contain at least one modification such as (1), a purine analogue, (2) a pyrimidine analogue, or (3) an analogous sugar, examples of such modified nucleotides being disclosed for example in the application PCT N^{o} WO 95/04 064.

For the purposes of the present invention, a first polynucleotide is considered as being “complementary” to a second polynucleotide when each base of the first polynucleotide is paired with a complementary base of the second polynucleotide running in the opposite direction. The complementary bases are A and T (or A and U), or C and G.

Without wishing to be bound by any specific theory, the inventors consider that the various characteristic units of the protein class according to the invention are such as to provide its biological function which is manifested by the observation of a phenotype of resistance against plant pathogens, particularly against the bacterium R. solanacearum.

Thus, the N-terminal portion, which contains the TIR, NB-ARC domains and one or more leucine-rich regions are characteristic of many pathogen resistance proteins in plants (R proteins), these proteins being assumed to activate the cascade signal metabolic routes which co-ordinate the initial defence responses of the plant which prevent progress of the pathogens.

The WRKY domain of the C-terminal portion of a protein according to the invention may have a binding function to regulatory sequences near to or within promoter regions of plant genes, the fixation of a protein according to the invention to DNA being thus able to modulate the activity of other genes potentially involved in the resistance of plants to pathogens, and particularly to R. solanacearum.

The applicant has isolated and characterized a nucleic acid coding for a polypeptide belonging to the class of plant pathogen resistance proteins defined above, protein RRS1-R.

More precisely, the applicant has isolated, from the genome of an Arabidopsis thaliana plant resistant to R. solanacearum, the RRS1-R gene which is able to provide to the plant a phenotype of resistance to R. solanacearum, such a nucleic acid comprising the sequence SEQ ID N^{o} 1.

The invention relates to a nucleic acid containing at least 15 consecutive nucleotides of a nucleotide sequence having at least 40% identity in nucleotides with a polynucleotide of sequence SEQ ID N^{o} 1, as well as to a nucleic acid of complementary sequence.

According to the invention, a first nucleic acid having at least 40% identity with a second reference nucleic acid, has at least 60%, preferably at least 80%, 85%, 90%, 95%, 98%, 99% or 99.5% identity in nucleotides with this second reference polynucleotide, the percentage identity between two sequences being determined as described below.

The “percentage identity” between two sequences of nucleotides or amino acids, in the context of the present invention, may be determined by comparing two optimally aligned sequences through a comparison window.

The portion of the nucleotide or polypeptide sequence in the comparison window may thus contain additions or deletions (for example “gaps”) compared to the reference sequence (which does not contain these additions or deletions) so as to obtain an optimal alignment of the two sequences.

The percentage is calculated by determining the number of positions in which an identical nucleic base or amino acid residue is observed for the two sequences (nucleic or peptide) compared, then dividing the number of positions in which there is identity between the two bases or amino acid residues by the total number of positions in the comparison window, then multiplying by one hundred to obtain the percentage identity of the sequence.

The optimal alignment of the sequences for the comparison is performed by computer using known algorithms contained in the software tool of the company WISCONSIN GENETICS SOFTWARE PACKAGE, GENETICS COMPUTER GROUP (GCG), 575 Science Doctor, Madison, Wis.

The percentage identity between two sequences is preferably performed using the BLAST software (version BLAST 2.06 of September 1998), using exclusively the default parameters (S. F. ALTSCHUL et al., (1990); S F ALTSCHUL et al., 1997).

The genomic sequence of the RRS1-R gene is referenced as the sequence SEQ ID N^{o} 1.

A further object of the invention is a nucleic acid which contains or comprises the sequence SEQ ID N^{o} 1.

The invention also relates to a nucleic acid containing at least 15 consecutive nucleotides of the polynucleotide of sequence SEQ ID N^{o} 1.

Any of the genomic nucleic acids according to the present invention may be readily obtained by a person skilled in the art who knows its nucleotide sequence disclosed in the present specification.

A man skilled in the art may also reproduce any of the genomic nucleic acids according to the invention by constructing, based on the sequences disclosed in the present specification, oligonucleotide primers able to amplify all or part of these nucleic acids from a plant genome, preferably an Arabidopsis thaliana genome or from a bank of vectors (YACS, BACS, cosrmids) containing genomic inserts of a plant, and preferably of Arabidopsis thaliana.

After amplification using appropriate primers, the various amplified nucleic acids are then subjected to a step of ligation in a plasmid vector so as to obtain the desired genomic nucleic acid, according to techniques well known to a person skilled in the art.

Moreover, the isolation of the DNA fragment containing the desired genomic insert may be obtained by sub-cloning from a bank (YACS, BACS, cosmids) containing genomic inserts of a plant, and preferably of Arabidopsis thaliana.

The sequence of the RRS1-R gene contains seven exons and six introns, as well as potential regulatory regions located respectively at the 5′ side of the first exon and on the 3′ side of the last exon, the structural characteristics of these exons and introns being detailed in tables 1 and 2 below.

TABLE 1
Sequence of the exons of the RRSI-R gene

| | POSITION OF THE 5′ | |
|---|---|---|
| EXON | NUCLEOTIDE IN SEQ ID | POSITION OF THE 3′ |
| N° | N° 1 | NUCLEOTIDE IN SEQ ID N° 1 |
| 1 | 260 | 636 |
| 2 | 746 | 1856 |
| 3 | 1937 | 2236 |
| 4 | 2326 | 3249 |
| 5 | 3438 | 4291 |
| 6 | 5377 | 5499 |
| 7 | 6085 | 6532 |

The invention also relates to a nucleic acid containing at least 15 consecutive nucleotides of an exonic polynucleotide of the RRS1-R gene, such as the polynucleotides 1 to 7 described in table 1 above, which are all included in the nucleic acid of sequence SEQ ID N^{o} 1.

In general, a nucleic acid having at least 15 consecutive nucleotides of a sequence according to the invention advantageously has at least 20, 25, 30, 35, 40, 50, 75, 100, 150, 200, 300, 400, 500, 1 000, 2 000, 3 000, 4 000 consecutive nucleotides of the reference sequence, the length of consecutive nucleotides being naturally limited by the length of the reference sequence.

Such a nucleic acid codes for at least a part of the RRS1-R polypeptide and may in particular be inserted in a recombinant vector intended for the expression of the corresponding translation product in a host cell or in a plant transformed with this recombinant vector.

Such a nucleic acid may also be used for the synthesis of nucleotide probes and primers intended for the detection or amplification of nucleotide sequences contained in the RRS1-R gene in a sample, or optionally of sequences of the RRS1-R genes carrying one or more mutations, preferably one or more mutations which would modify the phenotype of a plant carrying such a mutant RRS1-R gene, for example by modifying the regulation of mechanisms of resistance to pathogens, and preferably mechanisms or resistance to R. solanacearum.

TABLE 2
Regulatory and intron sequences of the
RRS-IR gene

| | Position of the 5′ nucleotide | Position of the 3′ nucleotide in |
|---|---|---|
| Intron n° | in SEQ ID N° 1 | SEQ ID N° 1 |
| 1 | 637 | 745 |
| 2 | 1857 | 1936 |
| 3 | 2237 | 2325 |
| 4 | 3250 | 3437 |
| 5 | 4292 | 5376 |
| 6 | 5500 | 6084 |

The invention also relates to a nucleic acid containing at least 15 consecutive nucleotides of an intronic polynucleotide of the RRS1-R gene, such as polynucleotides 1 to 6 described in table 2 above, which are all included in the nucleic acid of sequence SEQ ID N^{o} 1.

Such a nucleic acid may be used as an oligonucleotide probe or primer to detect the presence of at least one copy of the RRS1-R gene in a sample, or also to amplify a given target sequence within the RRS1-R gene.

In addition, the nucleic acid of the RRS1-R gene of sequence SEQ ID N^{o} 1 contains an ATG codon beginning at the nucleotide in position 260 (1st Exon) and a polyadenylation signal between the nucleotides in positions 6642 and 6647 inclusive.

The nucleic acid of the RRS1-R gene of sequence SEQ ID N^{o} 1 contains regulatory sequences located respectively on the 5′ side of exon 1 and the 3′ side of exon 7.

The 5′ regulatory region of the RRS1-R gene comprises the nucleotide sequence SEQ ID N^{o} 2.

The polynucleotide of sequence SEQ ID N^{o} 2 is located between the nucleotide in position 1 and the nucleotide in position 259 of sequence SEQ ID N^{o} 1.

The 3′ regulatory sequence of the RRS1-R gene comprises the nucleotide sequence SEQ ID N^{o} 3.

The polynucleotide of sequence SEQ ID N^{o} 3 is located between the nucleotide in position 6533 and the nucleotide in position 7936 of the nucleotide sequence SEQ ID N^{o} 1.

The polynucleotide derived from the regulatory regions of the RRS1-R gene above is useful for detecting the presence of at least one copy of this gene in a sample.

The invention also relates to a nucleic acid containing at least 15 consecutive nucleotides of a polynucleotide of sequence SEQ ID N^{o} 2 or SEQ ID N^{o} 3.

According to another embodiment, the invention also relates to a nucleic acid comprising a polynucleotide having at least 80% identity in nucleotides with a polynucleotide of sequence SEQ ID N^{o} 2 or SEQ ID N^{o} 3, advantageously at least 90%, 95%, 98%, 99%, 99.5% and more preferably 99.8% of identity in nucleotides with a polynucleotide selected from the sequences SEQ ID N^{o} 2 and SEQ ID N^{o} 3, or a biologically active fragment of the latter.

Preferred fragments of the nucleotide sequences SEQ ID N^{o} 2 or SEQ ID N^{o} 3 advantageously have a length of between 50, 100, 150, 200 and 300, 400, 600, 1 000 or 2 000 bases.

“Biologically active” fragment of a polynucleotide of sequence SEQ ID N^{o} 2 or SEQ ID N^{o} 3 according to the invention means a polynucleotide containing or consisting of a polynucleotide able to regulate the expression of a nucleic acid placed close to the latter, in a recombinant host cell.

For the purposes of the present invention, a nucleic acid constitutes a “functional” regulatory region to express a nucleic acid placed close to the latter, for example a nucleic acid coding for a polypeptide or a polynucleotide of interest, if this regulatory polynucleotide contains the nucleotide sequence containing the regulation signals of the transcriptions and/or the translation, and if the sequences are located in such a way as effectively to induce or increase the transcription or translation of said polypeptide or polynucleotide of interest.

In order to identify the “biologically active” fragments of the polynucleotides of sequence SEQ ID N^{o} 2 or SEQ ID N^{o} 3, a person skilled in the art may advantageously refer to the book by SAMBROOK et al. (1989) which describes the use of a recombinant vector carrying a marker gene (for example β-galactosidase, chloramphenicol acetyltransferase, etc.) whose expression is detected on placing the sequence of the marker gene under the control of the biologically active polynucleotide fragments of the sequences SEQ ID N^{o} 2 or SEQ ID N^{o} 3.

The genomic sequences located upstream of the first exon of the RRS1-R gene are cloned in an appropriate vector containing a marker gene, such as the GUS gene (Jefferson et al., 1987).

The polynucleotide fragments of sequence SEQ ID N^{o} 2 or SEQ ID N^{o} 3 according to the invention may be prepared from any of the sequences SEQ ID N^{o} 1, SEQ ID N^{o} 2 and SEQ ID N^{o} 3 by cleavage using appropriate restriction endonucleases, as described for example in the book by SAMBROOK et al. (1989) cited above.

The regulatory polynucleotide fragments according to the invention may also be prepared by digestion of any of the sequences SEQ ID N^{o} 1, SEQ ID N^{o} 2 and SEQ ID N^{o} 3 by an exonuclease enzyme, such as for example Bal3I as described by WABIKO et al. (1986), WABIKO H. et al., 1986, DNA, volume 5 (4): 305-314.

Such regulatory polynucleotides may also be prepared by chemical synthesis of the nucleic acids according to techniques well known to a person skilled in the art, such as the phosphoramidite techniques cited above.

The level of activity and tissue specificity of the regulatory sequences, in particular the promoter sequences of the RRS1-R gene according to the invention may be determined by measuring the level of expression of a detectable polynucleotide placed under the control of the latter in different types of plant cells and tissues. The detectable polynucleotide may be either a polynucleotide which is specifically hybridized with an oligonucleotide probe of predetermined sequence, or a polynucleotide coding a detectable protein, including the polypeptide RRS1-R or a fragment of this.

A test allowing such a verification is well known to a person skilled in the art and is in particular disclosed in the U.S. Pat. No. 5,502,176 and U.S. Pat. No. 5,266,488, incorporated herein by reference.

The invention thus also relates to a nucleic acid containing:

a) a nucleic acid comprising a regulatory nucleotide sequence of at least 50 consecutive nucleotides of the sequence SEQ ID N^{o} 3 or a sequence having at least 80% identity in nucleotides with sequence SEQ ID N^{o} 2;

b) a polynucleotide coding a polypeptide of interest or a polynucleotide of interest whose transcription and/or translation is placed under the control of the regulatory nucleic acid a);

c) optionally, a regulatory nucleic acid comprising at least 50 consecutive nucleotides of the sequence SEQ ID N^{o} 3 or a sequence having at least 80% identity in nucleotides with sequence SEQ ID N^{o} 3.

The polypeptide of interest coded by the nucleic acid b) above may be of various types and origins; it may be a protein of eukaryotic or prokaryotic origin.

The polypeptides of interest include the toxic polypeptides, such as for example the elicitins, for plant cells, their expression being such as to induce the death of the cell in which they are expressed and as a result a confinement of the pathogens which have infected the plant, thus preventing the propagation of the pathogen to all the organs of the plant. For example, it is possible to generate transgenic tobaccos having an increased resistance to the pathogens expressing eliciting, small toxic peptides produced by a phytopathogenic fungus, according to the technique described by Keller et al. (1999).

The nucleic acid of interest mentioned in b) above, generally an RNA molecule, may be complementary to a target polynucleotide, for example a polynucleotide located in a region coding the RRS1-R gene, and may thus be advantageously used as an antisense polynucleotide.

It has been shown according to the invention that the RRS1-R gene is transcribed in the form of a messenger RNA which has been isolated and characterized. This messenger RNA contains a unique open reading frame coding for the RRS1-R protein which is 1378 amino acids long.

The messenger RNA of the RRS1-R gene and the corresponding cDNA may be readily obtained by a skilled person in the art, for example by screening a cDNA band using a suitable probe or by rapid amplification of the cDNA ends (PCR, RACE-PCR). A person skilled in the art could also obtain the cDNA of the RRS1-R gene by using the techniques described in example 6, or by direct chemical synthesis.

A skilled person in the art could also reproduce a nucleic acid according to the invention by direct chemical synthesis, such as the phosphodiester method described by NARANG et al. (1979), the phosphodiester method described by BROWN et al. (1979), the diethylphosphoramidite method described by BEAUCAGE et al. (1981), as well as the solid phase method described in the European patent application EP 0 707 592, the content of these documents being incorporated herein by reference. On either side of the open reading frame, this messenger RNA contains respectively an untranslated 5′ region (5′-UTR) and an untranslated 3′ region (3′-UTR).

The cDNA resulting from the transcription of the RRS1-R gene is referenced as the sequence SEQ ID N^{o} 4.

The 5′-UTR sequence of the messenger RNA transcribed by the RRS1-R gene is composed of the sequence between the nucleotide in position 1 and the nucleotide in position 81 of the sequence SEQ ID N^{o} 4.

The 3′-UTR sequence of the messenger RNA transcribed by the RRS1-R gene is composed of the sequence between the nucleotide in position 4219 and the last nucleotide at the 3′-end of the sequence SEQ ID N^{o} 4.

The invention also relates to a nucleic acid containing at least 15 consecutive nucleotides of a polynucleotide selected from the 5′-UTR and 3′-UTR sequences of the messenger RNA of the RRS1-R gene described above, a polynucleotide having at least 80% identity in nucleotides with such a nucleic acid and a nucleic acid with complementary sequence to said nucleic acids or said polynucleotides.

A nucleic acid which contains or consists of the nucleotide sequence SEQ ID N^{o} 4 is also in the scope of the invention.

The 5′-UTR and 3′-UTR regions of the messenger RNA of the RRS1-R gene may contain components for regulating the transcription and/or translation of this gene, such as one or more ribosome binding sites, one or more polyadenylation sites, sequences enhancing the stability of the messenger RNA or all or part of the promoter region of the transcription.

The applicant has also isolated and characterized, from an ecotype of Arabidopsis thaliana sensitive to the R. solanacearum pathogen, the gene corresponding to the RRS1-R gene, containing, compared to the sequence of the RRS1-R gene, several additions and substitutions of nucleotides, the mutant gene found in the sensitive ecotype of Arabidopsis thaliana being designated RRS1-S for the purposes of the present specification.

The RRS1-S gene contains a first mutation in the portion coding for the C-terminal region of the protein, leading to the replacement of the amino acid sequence “SEASKLERL” between the amino acid in position 704 and the amino acid in position 712 of the polypeptide coded by the RRS1-R gene of sequence SEQ ID N^{o} 1 by the amino acid sequence “SEELERL” between the amino acid in position 704 and the amino acid in position 710 of the polypeptide coded by the RRS1-S gene. This mutation thus leads, on the one hand, to the substitution of the alanine residue in position 706 of the protein coded by the RRS1-R gene by a glutamic acid residue in position 706 of the protein coded by the RRS1-S gene and, on the other hand, by the deletion of the amino acids serine and lysine in positions 707 and 708 respectively of the amino acid sequence of the protein coded by the RRS1-R gene of sequence SEQ ID N^{o} 1.

A second mutation consists of the insertion of an early stop codon found in the coding region of the RRS1-S gene, leading to a deletion of 90 amino acids located at the C-terminal end of the RRS1-R protein, the polypeptide coded by the RRS1-S gene being as a result of 1 288 amino acids.

Without wishing to be bound by any particular theory, the applicant considers that the different mutations carried by the RRS1-S gene identified in the Col-5 ecotype of Arabidopsis thaliana, which is sensitive to the R. solanacearum pathogen, are responsible for the phenotype of sensitivity to R. solanacearum observed in this ecotype.

The result is that the different modifications observed in the nucleotide sequence of the RRS1-S gene compared to the nucleotide sequence of the RRS1-R gene are such as significantly to alter the biological activity of the resulting polypeptide.

In consequence, the nucleotide sequence of the RRS1-S gene found in the Col-5 ecotype of Arabidopsis thaliana is especially useful for developing various specific means for detection of the RRS1-S gene, such means of detection allowing a skilled person in the art to determine if a plant of interest contains the RRS1-S gene in its genome and is as a result sensitive to the bacterial pathogen R. solanacearum.

The genomic sequence of the RRS1-S gene is referenced as the sequence SEQ ID N^{o} 5. A further object of the invention is a nucleic acid which comprises or contains the sequence SEQ ID N^{o} 5.

The invention also relates to a nucleic acid containing at least 15 consecutive nucleotides of a polynucleotide of sequence SEQ ID N^{o} 5.

The invention further relates to a nucleic acid having at least 40% of identity in nucleotides with the nucleotide sequence SEQ ID N^{o} 5 or with a polynucleotide containing at least 15 consecutive nucleotides of the nucleotide sequence SEQ ID N^{o} 5, as well as a nucleic acid with complementary sequence.

The sequence of the RRS1-S gene contains 7 exons and 6 introns, whose structural characteristics are detailed in tables 3 and 4 below respectively.

TABLE 3
EXON SEQUENCES OF THE RRS1-S GENE

| | Position of the 5′ nucleotide in | Position of the 3′ nucleotide in |
|---|---|---|
| Exon n° | SEQ ID N° 5 | SEQ ID N° 5 |
| 1 | 1184 | 1560 |
| 2 | 1670 | 2780 |
| 3 | 2861 | 3160 |
| 4 | 3254 | 4171 |
| 5 | 4360 | 5213 |
| 6 | 6302 | 6424 |
| 7 | 6953 | 7136 |

The invention also relates to a nucleic acid containing at least 15 consecutive nucleotides of an exonic polynucleotide of the RRS1-S gene, such as the polynucleotides 1 to 7 described in table 3 above, which are all included in the nucleic acid of sequence SEQ ID N^{o} 5.

Such a nucleic acid codes for at least a part of the RRS1-S polypeptide and may in particular be inserted into a recombinant vector intended for the expression of the corresponding translation product in a host cell or in a plant transformed with this recombinant vector.

Such a nucleic acid may also be used for the synthesis of nucleotide probes and primers intended for the detection or amplification of nucleotide sequences contained in the RRS1-S gene in a sample.

TABLE 4
INTRON SEQUENCES OF THE RRS1-S GENE

| | Position of the 5′ nucleotide in | Position of the 3′ nucleotide in |
|---|---|---|
| Intron n° | SEQ ID N° 5 | SEQ ID N° 5 |
| 1 | 1561 | 1669 |
| 2 | 2781 | 2860 |
| 3 | 3161 | 3253 |
| 4 | 4172 | 4359 |
| 5 | 5214 | 6301 |
| 6 | 6425 | 6952 |

The invention also relates to a nucleic acid containing at least 15 consecutive nucleotides of an intronic polynucleotide of the RRS1-S gene, such as the polynucleotides 1 to 7 described in table 4 above, which are all included in the nucleic acid of sequence SEQ ID N^{o} 5.

Such a nucleic acid may be used as an oligonucleotide probe or primer to detect the presence of at least one copy of the RRS1-S gene in a sample, or to amplify a given target sequence within the RRS1-S gene.

Such probes are preferably constructed so as to hybridize specifically with regions of the RRS1-S gene which contain one or more substitutions, additions or deletions of bases compared to the nucleotide sequence of the RRS1-R gene of sequence SEQ ID N^{o} 1.

Such primers preferably allow the amplification of regions of the RRS1-S gene which contain one or more substitutions, additions or deletions of bases compared to the nucleotide sequence of the RRS1-R gene of sequence SEQ ID N^{o} 1.

The applicant has also isolated and characterized the untranscribed nucleotide sequences located respectively on the 5′ side and the 3′ side of the coding regions of the RRS1-S gene.

These untranscribed 5′ and 3′ sequences are able to carry the signals for regulating the transcription and/or translation of the RRS1-S gene and are also in the scope of the invention, and are respectively referenced as sequences SEQ ID N^{o} 6 and SEQ ID N^{o} 7.

The invention thus also relates to a nucleic acid containing at least 50 consecutive nucleotides of a regulatory polynucleotide of the RRS1-S gene selected from the nucleotide sequences SEQ ID N^{o} 6 and SEQ ID N^{o} 7 and the sequences having at least 80% identity in nucleotides with one of the sequences SEQ ID N^{o} 6 and SEQ ID N^{o} 7, as well as a nucleic acid with complementary sequence to said nucleic acids and polynucleotides.

The invention also relates to a nucleic acid containing:

a) a 5′ regulatory polynucleotide of the RRS1-S gene such as defined above;

b) a polynucleotide coding for a polypeptide or a polynucleotide of interest;

c) optionally, a 3′ regulatory polynucleotide of the RRS1-S gene such as defined above,

the nucleic acid coding for the polypeptide or the polynucleotide of interest being placed under the control of the 5′ regulatory polynucleotide of the RRS1-S gene and, optionally, also under the control of the 3′ regulatory polynucleotide of the RRS1-S gene.

The applicant has shown that the RRS1-S gene is transcribed in the form of a messenger RNA which has been isolated and characterized. This messenger RNA contains a unique open reading frame coding for the RRS1-S protein which is 1288 amino acids in length.

On either side of the open reading frame, this messenger RNA contains respectively a 5′ untranslated region (5′-UTR) and a 3′ untranslated region (3′UTR).

The cDNA of the RRS1-S gene is referenced as sequence SEQ ID N^{o} 8.

The 5′-UTR sequence of the messenger RNA transcribed by the RRS1-S gene is composed of the sequence between the nucleotide in position 1 and the nucleotide in position 81 of the sequence SEQ ID N^{o} 8.

The 3′-UTR sequence of the messenger RNA transcribed by the RRS1-S gene is composed of the sequence between the nucleotide in position 3949 and the last nucleotide at the 3′-end of the sequence SEQ ID N^{o} 8.

The invention also relates to a nucleic acid containing at least 15 consecutive nucleotides of a polynucleotide of sequence SEQ ID N^{o} 8 or 15 consecutive nucleotides of a sequence having at least 40% identity in nucleotides with sequence SEQ ID N^{o} 8, as well as a complementary sequence of said nucleic acid.

The nucleic acids according to the invention, and in particular the nucleotide sequences SEQ ID N^{o} 1 to SEQ ID N^{o} 8, their fragments of at least 15 nucleotides, the sequences having at least 40% identity in nucleotides with at least a part of the sequences SEQ ID N^{o} 1 to SEQ ID N^{o} 8, as well as the nucleic acids with complementary sequences, are useful for the detection of the presence of at least one copy of a nucleotide sequence of the RRS1-R or RRS1-S gene or a fragment or an allelic variant of these genes in a sample.

The nucleotide probes and primers which hybridize, under very strict hybridization conditions, with a nucleic acid selected from the sequences SEQ ID N^{o} 1 to SEQ ID N^{o} 8, are also in the scope of the invention.

The following hybridization conditions constitute very strict hybridization conditions in the context of the invention:

the DNA to be tested is immobilized on membranes of the Hybond-N⁺ type (Amersham, Buckinghamshire, UK) in accordance with the manufacturer's instructions, in the presence of 0.4 M NaOH overnight;

the membranes are washed with a buffer 5×SSC (1×SSC corresponds to 0.15 M NaCl+0.015 M sodium citrate), then the membranes are treated with UV at 312 nm for 3 minutes, then pre-hybridized for at least 4 hours at 65° C. in a hybridization buffer (6×SSC, 5× Denhardt's, 100 μg of calf thymus single-stranded DNA/ml and 0.5% SDS [weight/volume]).

The probes are added to the membranes and incubated at 65° C. overnight.

After the hybridization step, the membranes are washed in 500 ml of a buffer 2×SSC, 1% SDS (weight/volume) at laboratory temperature for 30 minutes;

a second washing is performed in 500 ml of a buffer 0.1×SSC, 0.1% SDS (weight/volume) for 15 minutes at 42° C.

The hybridization conditions described above are suitable for hybridization under very strict conditions of a nucleic acid molecule of 300 to 400 nucleotides in length.

Needless to say, the hybridization conditions described above may be adapted as a function of the length of the nucleic acid whose hybridization is sought or the type of marking selected, according to techniques known to a person skilled in the art.

Appropriate hybridization conditions may for example be adapted according to the teaching contained in the book by HAMES and HIGGINS (1985) or in that by AUSUBEL et al. (1989).

The nucleotide probes or primers according to the invention contain at least 15 consecutive nucleotides of a nucleic acid according to the invention, in particular of a nucleic acid of sequence SEQ ID N^{o} 1 to SEQ ID N^{o} 8 or its complementary sequence, of a nucleic acid having at least 40% identity in nucleotides with a sequence selected from the sequences SEQ ID N^{o} 1 to SEQ ID N^{o} 8 or its complementary sequence, or of a nucleic acid hybridizing, under very strict hybridization conditions, with a sequence selected from the sequences SEQ ID N^{o} 1 to SEQ ID N^{o} 8 or its complementary sequence.

The nucleotide probes or primers according to the invention preferably have a length of at least 15, 20, 25, 30, 35, 40, 50, 75, 100, 150, 200, 300, 400 or 500 consecutive nucleotides of a nucleic acid according to the invention, in particular of a nucleic acid with nucleotide sequence selected from the sequences SEQ ID N^{o} 1 to SEQ ID N^{o} 8, or of a nucleic acid of complementary sequence.

According to another embodiment a nucleotide probe or primer according to the invention contains or comprises fragments of a length of 15, 20, 25, 30, 35, 40, 50, 75, 100, 150, 200, 300, 400 or 500 consecutive nucleotides of a nucleic acid according to the invention, more particularly of a nucleic acid selected from the sequences SEQ ID N^{o} 1 to SEQ ID N^{o} 8, or of a nucleic acid of complementary sequence.

Examples or primers and primer pairs allowing amplification of different regions of the RRS1-R gene are for example the sequences SEQ ID N^{o} 11 to SEQ ID N^{o} 61 represented in table 6.

A nucleotide probe or primer according to the invention may be prepared by any suitable method known to a person skilled in the art, including cloning and action of restriction enzymes or by direct chemical synthesis according to techniques such as the phosphodiester method of Narang et al. (1979) or of Brown et al. (1979) cited above.

Any of the nucleic acids according to the invention, including the oligonucleotide probes and primers described above, may be marked, if desired, by incorporating a marker detectable by spectroscopic, photochemical, biochemical, immunochemical or chemical means.

For example, such markers may consist of radioactive isotopes (³²P,³H,¹³⁵S), fluorescent molecules (5-bromodeoxyuridine, fluorescein, acetylaminofluorene, digoxigenin) or ligands such as biotin.

The marking of a nucleic acid is preferably performed by incorporation of the marked molecules within nucleotides by extension of the primers, or by addition to the 5′ or 3′ ends.

Examples of non-radioactive marking of nucleic acid fragments are described in particular in the patent FR 78 19 175 or in the articles by URDEA et al. (1988), or SANCHEZ-PESCADOR et al. (1988).

The probes according to the invention may advantageously have structural properties enabling an amplification of the signal, such as the probes described by URDEA et al. (1991) or in the European patent n^{o} EP 0 225 807 (Chiron).

The oligonucleotide probes according to the invention may in particular be used in hybridizations of the Southern type to genomic DNA, or in hybridizations to the messenger RNA of the RRS1-R and RRS1-S genes, when the expression of the corresponding transcript is sought in a sample.

The probes according to the invention may also be used for the detection of PCR amplification products or also for the detection of mismatches.

Nucleotide probes or primers according to the invention may be immobilized on a solid phase. Such solid phases are well known to a person skilled in the art and include the surfaces of microtitration plate wells, polystyrene beads, magnetic beads, nitro-cellulose tapes or microparticles such as latex particles.

The present invention also relates to a method for detecting the presence of a nucleic acid of the RRS1-R or RRS1-S gene in a sample, said method comprising the steps of:

placing a nucleotide probe or several probes according to the invention with the sample to be tested;

detecting any complex which may be formed between the probes and the nucleic acid present in the sample.

According to a specific embodiment of the detection method according to the invention, the nucleotide probe or probes are immobilized on a solid phase.

According to a further embodiment, the oligonucleotide probes contain a detectable marker.

The invention also relates to a pack or kit for detecting the presence of a nucleic acid of the RRS1-R or RRS1-S gene in a sample, said kit comprising:

a) one or more nucleotide probes, such as described above;

b) optionally, the reagents necessary for the hybridization reaction.

According to a first embodiment, the detection pack or kit is characterized in that the probe or probes are immobilized on a solid phase.

According to a second embodiment, the detection pack or kit is characterized in that the oligonucleotide probes contain a detectable marker.

According to a particular embodiment of the detection kit described above, such a kit contains several oligonucleotide probes according to the invention which may be used to detect target sequences of interest of the RRS1-R or RRS1-S gene or to detect mutations of the coding or non-coding regions of the RRS1-R gene, more particularly the nucleic acids of the sequences SEQ ID N^{o} 1 to SEQ ID N^{o} 8 or the nucleic acids of complementary sequence.

The nucleotide primers according to the invention may be used to amplify any nucleotide fragment (gDNA, cDNA, mRNA) of RRS1-R or RRS1-S, and more particularly all or part of a nucleic acid of sequence SEQ ID N^{o} 1 to SEQ ID N^{o} 8.

Another object of the invention relates to a method for amplifying a nucleic acid of the RRS1-R or RRS1-S gene, and more particularly a nucleic acid of sequence SEQ ID N^{o} 1 to SEQ ID N^{o} 8 or a fragment or a nucleic acid of complementary sequence to the latter, contained in a sample, said method comprising the steps of:

a) placing the sample in which the presence of the target nucleic acid is suspected in contact with a pair of nucleotide primers whose position of hybridization is located respectively on the 5′ side and on the 3′ side of the region of the target nucleic acid of RRS1-R or RRS1-S whose amplification is sought, in the presence of the reagents necessary for the amplification reaction and;

b) detecting any nucleic acid amplified.

According to the amplification method above, at least one amplification cycle of the nucleic acid contained in the sample is performed before the detection of any nucleic acid amplified, preferably at least 10, and more preferably at least 20, cycles of amplification.

It is advantageous to use any one of the nucleotide primers described above to perform the above amplification method.

A further object of the invention is a pack or kit for amplifying a nucleic acid of the RRS1-R or RRS1-S gene according to the invention, and more particularly all or part of a nucleic acid of sequence SEQ ID N^{o} 1 to SEQ ID N^{o} 8, said pack or kit comprising:

a) a pair of nucleotide primers according to the invention, whose hybridization position is located respectively at the 5′ side and the 3′ side of the target nucleic acid of the RRS1-R or RRS1-S gene whose amplification is sought;

b) optionally, the reagents necessary for the amplification reaction.

Such an amplification pack or kit advantageously contains at least one pair of nucleotide primers such as described above.

According to a preferred embodiment, primers according to the invention contain all or part of a polynucleotide selected from the nucleotide sequences SEQ ID N^{o} 11 to SEQ ID N^{o} 61.

The applicant has shown that plants carrying the RRS1-R gene have a phenotype of resistance to R. solanacearum. In addition, the applicant has also shown that the insertion of the RRS1-R gene or of the cDNA of the RRS1-R gene into the genome of a plant initially sensitive to R. solanacearum provides to this plant a phenotype of resistance to this pathogen.

The invention also relates to methods and means designed to inhibit or block the expression of the RRS1-S gene found in plants sensitive to R. solanacearum, particularly with a view to increasing their resistance to different pathogens, by any technique known to a person skilled in the art.

In order to inhibit or block the expression of the RRS1-S gene in a plant, a skilled person in the art may use antisense polynucleotides.

Thus, the invention also relates to an antisense polynucleotide, able to hybridize specifically with a given region of the RRS1-S gene and able to inhibit or to block its transcription and/or translation. Such a polynucleotide has the general structure which has been defined above for the probes and primers according to the invention.

An antisense polynucleotide according to the invention preferably hybridizes with a sequence corresponding to a sequence located in the region of the 5′ end of the RRS1-S messenger RNA, and more preferably close to the codon for initiating the translation (ATG) of the RRS1-S gene.

According to a second preferred embodiment, an antisense polynucleotide according to the invention contains a sequence corresponding to one of the sequences located at the exon/intron junctions of the RRS1-S gene and preferably sequences corresponding to a splicing site, which may be determined according to techniques well known to a person skilled in the art, based on the description of the sequences of the RRS1-S gene of the present specification.

In order to synthesize the antisense polynucleotides such as defined above, a person skilled in the art may refer to tables 3 and 4 in which the positions of the different exons and introns of the RRS1-S gene in the sequence SEQ ID N^{o} 5 are listed.

The antisense polynucleotides must in general have a length and a melting point sufficient to form an intracellular duplex hybrid with a sufficient stability to inhibit the expression of the RRS1-S mRNA.

The strategies which may be used by a person skilled in the art to construct the antisense polynucleotides are particularly described by GREEN et al. (1986) and by IZANT and WEINTRAIB (1984), the content of these two articles being thus incorporated herein by reference.

Methods for construction of antisense polynucleotides which may be used by a person skilled in the art are also described by ROSSI et al. (1991) and in the PCT application n^{o} WO 94/23 026, WO 95/04141, WO 92/18522 and in the European patent application n^{o} EP 0 572 287, the content of these documents being incorporated by reference.

An antisense polynucleotide according to the invention advantageously has a length of 15 to 4 000 nucleotides. An antisense polynucleotide of the invention preferably has a length of between 15, 20, 25, 30, 35, 40, 45 or 50 to 75, 100, 200, 500, 1 000, 2 000, 3 000 or 4 000 nucleotides.

The preferred antisense polynucleotides according to the invention 10 are those having respectively a length of about 300 nucleotides or a length of about 4 000 nucleotides.

In order to inhibit or block the expression of the RRS1-S gene, it is possible simultaneously to use several antisense polynucleotides such as defined above, each of the antisense polynucleotides hybridizing with a specific region of the RRS1-S gene or of its messenger RNA.

The antisense polynucleotides according to the invention are more preferably defined in such a way that they hybridize with a region of the RRS1-S gene which has, in comparison with the corresponding sequence of the RRS1-R gene, one or more substitutions, deletions, additions of at least one base.

Other methods of using the antisense polynucleotides are for example those described by SCZAKIEL et al. (1995).

Another strategy for inhibiting or blocking the expression of the RRS1-S gene consists of using polynucleotides able to form a triple DNA helix with the genomic region of double-stranded DNA carrying the RRS1-S gene.

In general, homopurine sequences are considered as the most useful in this type of strategy, although homopyrimidine sequences may also be used.

A person skilled in the art may advantageously refer to the genomic sequence SEQ ID N^{o} 5 of RRS1-S in order to select in this sequence sequences of homopurine or of homopyrimidine of 10 to 20 nucleotides long, which are able to inhibit the expression of the RRS1-S gene.

Methods of inhibiting the expression of a gene by the triple helix technique are for example those described by GRIFFIN et al. (1989).

A further object of the invention is thus the use of an antisense polynucleotide or of a homopyrimidine polynucleotide, such as defined above, or of a recombinant vector containing such a polynucleotide, to inhibit or block the expression of the RRS1-S gene in a plant cell or in a whole plant.

As already noted, complementation experiments with Arabidopsis thaliana plants belonging to an ecotype sensitive to R. solanacearum by a recombinant vector containing the genomic sequence or a cDNA sequence of the RRS1-R gene revealed, in the transformed plant, the appearance of a phenotype of resistance to R. solanacearum.

In order to stimulate the resistance of a plant to different pathogens, and more particularly to R. solanacearum, it would thus be advantageous either to introduce or to increase the number of copies of the RRS1-R gene in this plant, or to encourage the expression of the RRS1-R gene, these two methods being likely to prevent the development of such a pathogen.

A strong expression of the RRS1-R gene in a plant may be achieved either by overexpression of the RRS1-R gene, or by insertion of multiple copies of a polynucleotide coding for the RRS1-R protein into the plant, or by a combination of these two strategies. For the insertion of multiple copies of a polynucleotide coding for the RRS1-R protein into the genome of a plant, the use of a recombinant vector according to the invention is advantageous.

The invention also relates to a recombinant vector containing a nucleic acid according to the invention.

Such a recombinant vector advantageously contains a nucleic acid selected from among the following nucleic acids:

a nucleic acid containing at least 15 consecutive nucleotides of a nucleotide sequence coding for a protein for resistance of a plant to a pathogen, said protein comprising:

(i) an N-terminal portion containing at least one sequence of amino acids rich in leucine and at least one nucleotide binding site; and

(ii) a C-terminal portion containing a DNA binding domain comprising the amino acid sequence “WRKYGQK”, as well as a nucleic acid of complementary sequence;

b) a nucleic acid containing a sequence having at least 40% identity in nucleotides with the nucleotide sequence SEQ ID N^{o} 1, as well as a nucleic acid of complementary sequence;

c) a nucleic acid containing a sequence having at least 40% identity in nucleotides with the nucleotide sequence SEQ ID N^{o} 5;

d) a nucleic acid containing at least 15 consecutive nucleotides of a polynucleotide consisting of one of the exons 1 to 7 of the RRS1-R gene or of the RRS1-S gene, such as defined above;

e) a nucleic acid comprising a polynucleotide of at least 15 consecutive nucleotides of a polynucleotide consisting of one of the introns 1 to 7 of the RRS1-R or RRS1-S gene, such as defined above;

f) a nucleic acid having at least 15 consecutive nucleotides of a regulatory polynucleotide of the RRS1-R or RRS1-S gene, such as defined above;

g) an antisense polynucleotide or a homopurine or homopyrimidine polynucleotide, such as defined above, useful for inhibiting the expression of the RRS1-S gene;

“Vector”, in the sense of the present invention, means a circular or linear molecule of DNA or RNA which may be either in single or double-stranded form.

A recombinant vector according to the invention may be either a cloning vector, an expression vector, or more specifically an insertion vector, a transformation vector or an integration vector.

It may be a vector of bacterial or viral origin.

According to a first embodiment, a recombinant vector according to the invention is used with the aim of amplifying the nucleic acid which is inserted after transformation or transfection of the desired host cell.

According to a second embodiment, the vector is an expression vector containing, in addition to a nucleic acid coding for a polypeptide according to the invention, in particular polypeptides coded by the RRS1-R and RRS1-S genes, regulatory sequences allowing directing the transcription and/or the translation.

According to an advantageous embodiment, a recombinant vector according to the invention particularly contains the following components:

1) components regulating the expression of the nucleic acid of the RRS1-R gene or of the RRS1-S gene to be inserted, such as promoters and enhancer sequences;

2) the sequence contained in the nucleic acid of the RRS1-R or RRS1-S gene according to the invention to be inserted in such a vector, said sequence being placed under the control of the regulation signals described in (1); and

3) initiation and stop sequences of the appropriate transcription.

In addition, the recombinant vectors according to the invention may include one or more replication origins in the host cells in which their amplification or their expression is sought as well as selection markers.

In a particular embodiment, a recombinant vector according to the invention contains an antisense polynucleotide or a homopurine or homopyridine polynucleotide, such as defined above, optionally placed under the control of suitable regulatory sequences ensuring the expression in a selected host cell or plant. Such a recombinant vector is preferably used to inhibit the expression of the RRS1-S gene in the cell or in the plant.

According to another particular embodiment, a recombinant vector according to the invention contains a polynucleotide coding for the RRS1-R polypeptide or a polypeptide having at least 40% identity in amino acids with the latter and retaining the biological activity of RRS1-R, placed under the control of regulatory sequence(s) enabling strong expression of RRS1-R or of its homologue in a selected host cell or plant. Such a recombinant vector is useful to enable a high level of expression of RRS1-R in a plant.

According to an advantageous embodiment, such a recombinant vector is an integrative vector enabling the insertion of multiple copies of the coding sequence of RRS1-R in the genome of a plant.

As an example, the bacterial promoters may be the promoters LacI, LacZ, promoters of the RNA polymerase of the bacteriophage T3 or T7, the promoters PR, or PL of the phage lambda.

Promoters for the expression of a nucleic acid of RRS1-R or RRS1-S according to the invention in plants are the promoter CaMV 35 S of the cauliflower mosaic virus of Odell et al. (1985), or the promoter of the gene of lactin 1 of rice, McElroy et al. (1990).

Other promoters useful to a person skilled in the art for the expression of a polynucleotide of interest in plants are described in the U.S. Pat. Nos. 5,750,866 and 5,633,363, incorporated herein by reference.

In general, for the choice of a suitable promoter, a person skilled in the art may advantageously refer to the book by Sambrook et al. (1989) cited above or the techniques described by Fuller et al. (1996), and Ausubel et al. (1989).

The preferred bacterial vectors according to the invention are for example the vectors pBR 322 (ATCC N^{o} 37017) or the vectors such as pAA223-3 (Pharmacia Uppsala, Sweden) and pGEM1 (Promega Biotech, Madison, WU, USA) and pUC19 (marketed by Boehringer Mannheim, Germany).

Other commercially available vectors include the vectors pQE70, pQE60, pQE9 (Qiagen, psuX 174, pBluescript SA, pNH8A, pMH16A, pMH18A, pMH46A, pWLNEO, pSG2CAT, pOG44, pXT1, pSG (Stratagene).

They may also be vectors of the baculovirus type such as the vector pVL1392/1393 (Pharmingen) used to transfect the cells of the line Sf9 (ATC N^{o} CRL 1711) derived from Spodoplera frugidera.

It is preferable to use vectors especially suitable for the expression of the sequence of interest in plant cells, such as the following vectors:

vector pBIN19 (Bevan et al., Nucleic Acids Research, Vol. 12: 8711-8721, marketed by the Company Clontech, Palo Alto, Calif., USA);

vector pBI101 (Jefferson 1987, Plant Molecular Biology Reporter, vol. 5 : 387-405, marketed by the Company Clontech);

vector pBI121 (Jefferson et al. 1987, Plant Molecular Biology Reporter, vol. 5 : 387-405, marketed by the Company Clontech);

vector pEGFP (Cormack BP et al., 1996, marketed by the Company Clontech);

vectors SLJ75515 and SLJ75516 (gift from Dr. J. Jones, The Sainsbury Laboratory, Norwich, UK);

vectors pDHB321 (gift from Dr. Bouchez, INRA Versailles, France);

vectors pAOV, pOV2, pSOV, pSOV2, pkMB and pSMB (Mylne et al., 1996).

In order that they can express the polynucleotides according to the invention, these vectors must be introduced into a host cell. The introduction of the polynucleotides according to the invention into a host cell may be performed in vitro, according to techniques well known to a person skilled in the art for transforming or transfecting the cells, either in primary culture, or in the form of cell lines.

A further object of the invention is a host cell transformed with a nucleic acid or by a recombinant vector according to the invention.

Such a transformed host cell is preferably of prokaryotic or eukaryotic origin, especially bacterial, fungal or plant.

Bacterial cells which may particularly be used are from different strains of E. coli or of Agrobacterium tumefaciens.

The transformed host cell is preferably a plant cell or a plant protoplast.

Even more preferably, it is a cell or a protoplast of rape, tobacco, corn, tomato, potato or of Arabidopsis thaliana.

The invention also relates to a transformed multicellular plant organism, characterized in that it comprises a transformed host cell or a multiplicity of host cells transformed with a nucleic acid of the RRS1-R or RRS1-S gene or with a recombinant vector according to the invention.

According to a first embodiment, the multicellular plant organism is transformed with one or more antisense nucleotides and/or one or more homopurine or homopyrimidine polynucleotides so as to inhibit or block the expression of the RRS1-S gene in this organism.

According to a second embodiment, the multicellular plant organism is transformed with one or more copies of a polynucleotide coding for the RRS1-R protein or for a polypeptide having at least 40% identity in amino acids with the RRS1-R polypeptide and retaining its biological activity, enabling it to provide a phenotype of resistance to R. solanacearum to the transformed organism.

A further object of the invention is a transgenic plant, in other words a transformed plant containing, preferably in a form integrated into its genome, a nucleic acid of the RRS1-R or RRS1-S gene and preferably an antisense polynucleotide or a homopurine or homopyrimidine polynucleotide or a nucleic acid coding for the RRS1-S polypeptide or a homologous polypeptide, said nucleic acid having been inserted into the genome of the plant by transformation with a nucleic acid of RRS1-R or RRS1-S or a recombinant vector according to the invention.

A transformed plant according to the invention is preferably a rape, tobacco, corn, tomato, potato or Arabidopsis thaliana plant.

According to a first embodiment, the transgenic plants such as defined above have a reduced, undetectable or absence of expression of the RRS1-S gene and are thus able to show increased resistance to pathogens such as R. solanacearum.

According to a second embodiment, the transgenic plants such as defined above have the property of strongly expressing the RRS1-R polypeptide and thus having a phenotype of resistance to various pathogens, and particularly to bacterial pathogens such as R. solanacearum.

A further object of the invention is a method for obtaining a transgenic plant transformed with a nucleic acid according to the invention, characterized in that it comprises the following steps:

a) obtaining a plant recombinant host cell such as defined above;

b) regenerating an entire plant from the transformed plant host cell obtained in step a);

c) selecting the plants obtained in step b) which have integrated the nucleic acid of the RRS1-R or RRS1-S gene of interest.

The invention also relates to a method for obtaining a transgenic plant, transformed with a nucleic acid according to the invention, characterized in that it comprises the following steps:

a) transforming a plant cell with a nucleic acid of the RRS1-R or RRS1-S gene or with a recombinant vector according to the invention;

b) regenerating an entire plant from cells of transformed plants obtained in step a);

c) selecting the plants which have integrated the nucleic acid of the RRS1-R or RRS1-S gene of interest.

The invention also relates to a method for obtaining a transformed plant, characterized in that it comprises the following steps:

a) obtaining a host cell of Agrobacterium tumefaciens transformed with a nucleic acid or a recombinant vector according to the invention;

b) transforming the selected plant by infection with the cells of Agrobacterium tumefaciens obtained in step a);

c) selecting the plants which have integrated the nucleic acid according to the invention.

Any of the methods described above for obtaining a transgenic plant may also contain the following additional steps:

d) crossing between themselves two transformed plants such as obtained in step c);

e) selecting the plants homozygous for the transgene.

According to another embodiment, any of the methods described above may also contain the following steps:

f) crossing a transformed plant obtained in step c) with a plant of the same species;

g) selecting the plants arising from the crossing of step d) which have retained the transgene.

A person skilled in the art is able to use many methods of the state of the art in order to obtain plants transformed with a nucleic acid of the RRS1-R or RRS1-S gene according to the invention.

A skilled person in the art may advantageously refer to the technique described by BECHTOLD et al. (1993) in order to transform a plant using the bacterium Agrobacterium tumefaciens.

The techniques used in other types of vectors may also be used, such as the techniques described by BOUCHEZ et al. (1993) or by HORSCH et al. (1984).

Another object of the invention is a transformed plant such as obtained by any of the methods for obtaining it described above.

The invention also relates to a plant seed whose component cells contain a nucleic acid of the RRS1-R gene or of the RRS1-S gene according to the invention which has been artificially inserted in their genome.

Another object of the invention is a seed of a transgenic plant such as defined above.

A further object of the invention consists of the use of a nucleic acid of the RRS1-R gene or of the RRS1-S gene according to the invention for in vitro or in vivo expression, preferably in planta of the RRS1-R or RRS1-S protein or of a peptide fragment of this.

The invention also relates to the use of an antisense nucleic acid, or of a homopurine or homopyrimidine nucleic acid according to the invention to inhibit or block the expression of the gene coding for the RRS1-R or RRS1-S protein.

The above uses are preferably characterized in that they consist of an in vivo expression in a plant transformed with such a nucleic acid.

As already stated above, the RRS1-R gene codes for a polypeptide of 1378 amino acids in length.

In addition, the RRS1-R polypeptide shows structural properties defined above in the specification, i.e. the characteristic presence of functional domains found in combination for the first time in the amino acid sequence of a polypeptide, which provides to the RRS1-R protein the status of being the first member of a new class of proteins defined in the most general way as proteins containing:

a) an N-terminal portion containing at least one sequence of amino acids rich in leucine and at least one nucleotide binding site; and

b) a C-terminal portion containing a DNA binding domain containing the amino acid sequence “WRKYGQK”.

The RRS1-R polypeptide is referenced as the sequence SEQ ID N^{o} 9.

As already noted, the applicant has shown that the mutations in the amino acid sequence of the RRS1-R polypeptide can lead to a protein devoid of the biological activity of the wild RRS1-R protein, the expression of the mutant protein in the plant no longer enabling the observation of the phenotype of resistance to certain pathogens, such as R. solanacearum. A representative polypeptide of the mutant polypeptides of RRS1-R modified in their biological function is the RRS1-S polypeptide whose amino acid sequence is referenced as the sequence SEQ ID N^{o} 10.

According to another embodiment, the invention also relates to a polypeptide coded by a nucleic acid of the RRS1-R gene or of the RRS1-S gene, and preferably a polypeptide containing at least 5 consecutive amino acids of a protein selected from RRS1-R and RRS1-S.

Such a polypeptide preferably contains at least 10, 15, 20, 25, 30, 40, 50, 60, 70, 80, 90, 100, 150, 200, 250, 300, 350, 400, 450, 500, 600, 700, 1 200 consecutive amino acids of the RRS1-R polypeptide of amino acid sequence SEQ ID N^{o} 9 or of the RRS1-S polypeptide of amino acid sequence SEQ ID N^{o} 10.

The invention also relates to a polypeptide containing amino acid sequences having at least 40% identity in amino acids with the sequence of the RRS1-R polypeptide SEQ ID N^{o} 9, with the sequence of the RRS1-S polypeptide SEQ ID N^{o} 10, or of a peptide fragment of these.

Advantageously included in the invention is a polypeptide having at least 60%, 80%, 85%, 90%, 95% or 99% identity in amino acids with the sequence of the RRS1-R polypeptide of SEQ ID N^{o} 9, with the sequence of the RRS1-S polypeptide SEQ ID N^{o} 10, or of a peptide fragment of these.

In general, the polypeptides according to the invention are in an isolated or purified form.

The invention also relates to a method for producing the RRS1-R polypeptide of sequence SEQ ID N^{o} 9, the RRS1-S polypeptide of sequence SEQ ID N^{o} 10 or a peptide fragment of these, the method comprising the steps of:

a) inserting a nucleic acid coding for the RRS1-R, RRS1-S polypeptide or a peptide fragment of these, into an appropriate vector;

b) culturing, in an appropriate culture medium, a host cell previously transformed or transfected with the recombinant vector of step a);

c) recovering the transformed host cell from the treated or lysed culture medium, for example by sonication or osmotic shock;

d) separating and purifying said polypeptide from the culture medium or cell lysates obtained in step c);

e) optionally, characterizing the recombinant polypeptide produced.

The peptides according to the invention may be characterized by fixation on an immunoaffinity chromatographic column on which antibodies directed against these polypeptides or a fragment or a variant of these polypeptides have been previously immobilized.

According to another embodiment, a recombinant polypeptide according to the invention may be purified by passage through a chromatography column according to methods known to a person skilled in the art and described for example by AUSUBEL F. et al. (1989) cited above.

A polypeptide according to the invention may also be prepared by conventional chemical synthesis techniques either in homogeneous solution or in the solid phase.

As an illustrative example, a polypeptide according to the invention 10 may be prepared by the homogenous solution technique described by HOUBEN WEYL (1974) or by the solid phase synthesis technique described by MERRIFIELD (1965a, 1965b).

Polypeptides referred to as homologues of the RRS1-R or RRS1-S polypeptides, or of their fragments, are also in the scope of the invention.

Such homologous polypeptides have amino acid sequences having one or more substitutions of an amino acid by an equivalent amino acid, compared to the reference polypeptide.

In the context of the present invention, equivalent amino acids should be understood to mean for example the replacement of a residue in the L form by a residue in the D form, or the replacement of a glutamic acid (E) by a pyro-glutamic acid according to techniques well known to a person skilled in the art.

As an illustration, the synthesis of peptides containing at least one residue in the D form is described by KOCH et al. (1977).

According to another embodiment, other examples of equivalent amino acids are two amino acids belonging to the same class, in other words two acid, basic, non-polar or polar uncharged amino acids.

Also in the scope of the invention is a polypeptide containing amino acid modifications of 1, 2, 3, 4, 5, 10 to 20 substitutions, additions or deletions of an amino acid compared to the amino acid sequence of the RRS1-R polypeptide or of the RRS1-S polypeptide according to the invention.

The polypeptides according to the invention preferably containing one or more additions, deletions, substitutions of at least one amino acid retain their ability to induce the resistance to various pathogens, and particularly to R. solanacearum.

According to another preferred embodiment, the polypeptides according to the invention containing one or more additions, deletions, substitutions of at least one amino acid retain their capacity to be recognized by antibodies directed against the unmodified RRS1-R or RRS1-S polypeptides.

A polypeptide derived from the RRS1-R protein or from the RRS1-S protein is particularly useful for the preparation of antibodies intended for the detection of the presence of one or the other of these polypeptides or of a peptide fragment of them in a sample.

In addition to the detection of the presence of the RRS1-R or RRS1-S polypeptide or of a peptide fragment of them in a sample, antibodies directed against these polypeptides are used to quantify the synthesis of RRS1-R or RRS1-S, for example in plant cells, and thus to determine the capacity of this plant to resist certain pathogens, particularly R. solanacearum.

The preferred antibodies according to the invention are antibodies specifically recognizing the amino acid sequence from the amino acid in position 704 to the amino acid in position 712 of the sequence of the RRS1-R polypeptide of SEQ ID N^{o} 9.

A second class of preferred antibodies according to the invention is the antibodies specifically recognizing the amino acid sequence from the amino acid in position 704 to the amino acid in position 710 of the sequence of the RRS1-S polypeptide of SEQ ID N^{o} 10.

A third class of preferred antibodies according to the invention is the antibodies specifically recognizing the amino acid sequence from the amino acid in position 1291 to the amino acid in position 1378 of the RRS1-R polypeptide of sequence SEQ ID N^{o} 9.

By “antibody” in the context of the present invention should be in particular understood polyclonal or monoclonal antibodies or their fragments (for example the fragments F(ab)′₂, F(ab)) or any polypeptide containing an initial antibody domain recognizing the target polypeptide or polypeptide fragment according to the invention.

The monoclonal antibodies may be prepared from hybridomas according to the technique described by KOHLER and MILSTEIN (1975).

The present invention also relates to antibodies directed against a polypeptide such as described above or a fragment or a variant of this, such as produced in the trioma technique or the hybridoma technique described by KOZBOR et al. (1983).

The invention also relates to single chain antibody Fv (ScFv) such as described in the U.S. Pat. No. 4,946,768 or by MARTINEAU et al. (1998).

The antibodies according to the invention also include the antibody fragments obtained using phage banks (RIDDER et al., 1995), REINMANN K. A. et al., 1997).

The antibody preparations according to the invention are useful in immunological detection tests intended for the identification of the presence and/or the quantity of the RRS1-R or RRS1-S protein or a peptide fragment of one of these proteins in a sample.

An antibody according to the invention may additionally contain a detectable isotopic or non-isotopic, for example fluorescent, marker, or may be coupled with a molecule such as biotin, according to techniques well known to a person skilled in the art.

A further object of the invention is thus a method for detecting the presence of a RRS1-R polypeptide, a RRS1-S polypeptide or a peptide fragment of one of these polypeptides according to the invention, in a sample, said method comprising the steps of:

a) placing the sample to be tested in contact with an antibody such as defined above;

b) detecting the antigen/antibody complex formed.

The invention also relates to a diagnostic pack or kit for detecting the presence of a polypeptide according to the invention in a sample, said kit comprising:

a) an antibody such as defined above;

b) optionally, a reagent allowing the detection of the antigen/antibody complexes formed.

The invention also relates to fusion proteins containing an amino acid sequence of one of the two C-terminal or N-terminal functional domains of a polypeptide according to the invention, on the one hand, and on the other hand all or part of the amino acid sequence of a heterologous polypeptide.

In the context of the present specification, “heterologous polypeptide” should be understood as an amino acid sequence which is not naturally present in the amino acid sequence of the polypeptide or of the N-terminal or C-terminal fragment of the polypeptide according to the invention with which this “heterologous” amino acid sequence is covalently bonded, preferably by a peptide bond.

According to a first embodiment, a fusion protein or chimera according to the invention contains (1) the N-terminal domain of a first polypeptide belonging to the new class of proteins of resistance to plant pathogens according to the invention, covalently bonded, preferably by a normal peptide bond, to a second polypeptide containing the C-terminal domain of a second protein also belonging to the class of proteins of resistance to plant pathogens according to the invention.

The N-terminal domain of the first polypeptide and the C-terminal domain of the second polypeptide may be directly bonded to each other by a peptide bond.

According to a second embodiment, the N-terminal domain of the first polypeptide and the C-terminal domain of the second polypeptide may be separated, within the fusion protein, by a spacer amino acid sequence which may be of any type.

Such a chimeric protein thus contains an N-terminal domain containing characteristic units of a protein of resistance to pathogens, covalently bound to a C-terminal domain containing a nucleotide binding site likely to have a different specificity to that of the C-terminal domain naturally found within the amino acid sequence of the natural polypeptide from which the N-terminal domain of the chimeric protein originates.

Such chimeric proteins constitute new means enabling, by suitable choice of the N-terminal domain, a resistance to a given pathogen to be provided to a plant.

In addition, the choice of the C-terminal domain for binding to nucleotides allows a person skilled in the art to target specifically the regulatory sequences which are activated by such a chimeric protein and thus to control both the type and level of resistance desired.

According to a second embodiment, a fusion protein according to the invention consists of (1) the amino acid sequence of all or part of a protein of which the mechanisms of induction in a plant are known and (2) (i) either the complete amino acid sequence of a polypeptide of resistance to pathogens according to the invention, or (ii) the amino acid sequence of the C-terminal domain containing a nucleotide binding site of a polypeptide according to the invention.

The synthesis of such a protein is preferably inducible by stress signals of the plant, such as a stress due to a sudden change in the temperature of the environment.

The invention also relates to a nucleic acid coding for a chimeric protein such as defined above.

According to a preferred embodiment, such a nucleic acid contains, close to and advantageously upstream of the coding region, a constitutive or inducible promoter.

As an illustration, such a nucleic acid contains, from the 5′ end to the 3′ end, a plant heat shock promoter protein, (2) a nucleotide sequence coding for the N-terminal portion of the heat shock protein whose synthesis is naturally regulated by the promoter (1), (3) the nucleotide sequence coding a polypeptide of resistance to plant pathogens which has retained its transcription factor function (WRKY functional domain) according to the invention.

As an example, the preferred promoters are those which are inducible by:

Heat (Athsp 17.6; Prandl et al., 1995);

H₂O₂, auxin, salicylic acid (GST6; Chen et al., 1996);

salicylic acid (PR1; Lebel et al., 1998).

A chimeric protein according to the invention may be obtained according to the technique described in example 7.

As has been described above, a polypeptide of resistance to plant pathogens according to the invention, has, both in the N-terminal domain and in the C-terminal domain, characteristic units of binding to proteins.

Proteins interacting with a polypeptide according to the invention thus also represent means involved in the resistance of plants to infection by certain pathogens, particularly by R. solanacearum.

An additional object of the invention is methods and means designed for the screening of substances or molecules which may be candidates for binding with a polypeptide according to the invention.

The invention thus relates to a method for screening a candidate substance fixing to a polypeptide according to the invention, and more particularly a RRS1-R or RRS1-S polypeptide, characterized in that it comprises the steps of:

a) preparing a polypeptide according to the invention;

b) obtaining a candidate substance to be tested;

c) placing the polypeptide of step a) in contact with the candidate substance of step b);

d) detecting any complex formed between the polypeptide of the invention and the candidate substance.

As an illustration, in a screening method as defined above, a polypeptide according to the invention is immobilized by adsorption or by covalent bonding on an appropriate surface, the surface of a microtitration plate well. The immobilized polypeptide of the invention is then placed in contact with the candidate substance or molecule to be tested.

The detection of the fixation of the candidate molecule on the immobilized polypeptide of the invention may then be performed for example with an antibody specifically recognizing the candidate molecule to be tested, said antibody containing optionally a detectable marker.

According to another embodiment of a screening method such as defined above, the candidate substance or molecule is previously immobilized on the surface of a solid phase before being brought into contact with a polypeptide according to the invention.

The detection of any complex formed between the polypeptide according to the invention and the candidate molecule immobilized on the solid phase may then be performed using an antibody specifically recognizing a polypeptide according to the invention, said antibody optionally being marked.

To perform such a screening method, a person skilled in the art may advantageously refer to the use of immunodetection techniques of the ELISA type.

The invention also relates to a kit or pack for screening a candidate substance fixing to a polypeptide of the invention, such kit or pack comprising:

a) a polypeptide according to the invention;

b) optionally, the reagents necessary for the detection of the complexes formed between the polypeptide of the invention and the candidate substance to be tested.

A method for screening molecules interacting with a polypeptide according to the invention may also be performed according to double hybrid techniques. Double hybrid screening techniques are used to study protein-protein interactions in vivo and are based on the fusion of a “bait” protein with the DNA binding site of the yeast protein Gal4.

The double hybrid technique is particularly described in the U.S. Pat. Nos. 5,667,973 and 5,283,173, the technical teaching of these two patents being incorporated herein by reference.

The screening of bands of cDNA containing inserts coding for translation products potentially interacting with a polypeptide according to the invention or a peptide fragment containing a domain binding with proteins of a polypeptide according to the invention may be performed as described by HARPER et al. (1993), by CHO et al. (1998), or by FROMONT-RACINE et al. (1997).

The invention also relates to a method for screening a candidate substance fixing to a polypeptide according to the invention, and more particularly to the RRS1-R or RRS1-S polypeptide, said method comprising the steps of:

a) obtaining a first nucleic acid coding for a fusion protein containing a part of the polypeptide of interest fused to the DNA binding site of a transcription factor protein such as Gal4;

b) obtaining a second nucleic acid coding for a fusion protein containing the candidate substance fused to the transcription domain of a transcription factor protein such as Gal4;

c) producing a nucleic acid containing a nucleotide sequence coding for a detectable marker, placed under the control of a regulatory sequence recognized by the transcription factor protein such as Gal4.

The nucleic acids a), b) and c) being inserted into appropriate vectors, and d) co-transfected with yeast simultaneously with said vectors;

e) detecting the expression of the nucleotide sequence coding for the detectable marker.

The screening method above may additionally include a step f) during which the nucleotide sequence and/or the amino acid sequence of the “prey” nucleic acid inducing the expression of the detectable marker is characterized.

The invention also relates to a kit or pack for screening a candidate substance fixing to a polypeptide according to the invention, and more particularly the RRS1-R or RRS1-S polypeptide, said kit or pack comprising:

a) a first nucleic acid coding for a fusion protein containing a portion of the polypeptide of interest fused to the DNA binding domain of a transcription factor such as Gal4;

b) optionally, a second nucleic acid containing a nucleotide sequence coding for a detectable marker, placed under the control of a regulatory sequence recognized by the transcription factor such as Gal4;

c) a third nucleic acid coding for a fusion protein containing the candidate substance fused to the transcription domain of a transcription factor such as Gal4.

The invention also relates to a substance able to fix to a polypeptide of the invention, preferably to the RRS1-R or RRS1-S polypeptide, this substance being characterized in that it is able to be obtained by a screening method such as that defined above.

A screening method making use of a system of the double hybrid type may be readily performed by a person skilled in the art, particularly according to the technique of example 8.

As already described above, a polypeptide according to the invention contains several nucleotide binding domains both in its N-terminal portion and in its C-terminal portion. The nucleotide sequences to which these different nucleotide binding sites bind thus assume a critical importance in the mechanisms of resistance of plants to different pathogens, and more particularly to R. solanacearum.

The isolation and characterization of the polypeptides of resistance to plant pathogens according to the invention now allow a skilled person in the art to determine these nucleotide sequences involved in the induction of a phenotype of resistance to pathogens in a plant.

The invention also relates to a method of screening a nucleic acid interacting with a polypeptide according to the invention, said screening method comprising the steps of:

a) obtaining a statistical population of nucleic acids of 20 to 50 nucleotides in length;

b) placing the statistical population of nucleic acids of step a) in contact with a polypeptide according to the invention;

c) characterizing the nucleic acid or acids interacting with said polypeptide.

According to such a method, a population of nucleic acids is first prepared, for example by direct chemical synthesis, which are from 20 to 50 nucleotides in length, and preferably from 20 to 30 nucleotides in length, whose nucleotide sequence is statistical, each of the synthetic nucleic acids being additionally covalently bound, at their 5′ or 3′ end, to a unique known nucleotide sequence.

The nucleic acids forming a statistical population of nucleic acids described above are then placed in contact with a polypeptide according to the invention, said polypeptide optionally being previously immobilized on a solid phase.

After a step of removing the nucleic acids which have not interacted with the polypeptide according to the invention, the polypeptide/nucleic acid complexes are placed in contact with an oligonucleotide primer specifically hybridizing, under given hybridization conditions, with the unique known nucleotide sequence common to all the nucleic acids of the statistical population above, then follows at least one amplification cycle using the primer hybridizing with the unique known nucleotide sequence of the nucleic acid retained by the polypeptide of the invention, for example an amplification by PCR.

According to a particular embodiment of this screening method, the nucleic acids thus amplified and isolated are again placed in contact with a polypeptide according to the invention, then the nucleic acid or acids which have formed a complex with said polypeptide are again hybridized with the nucleotide primer hybridizing with the unique known nucleotide sequence and a new cycle or cycles of amplification are performed.

The screening method such as defined above preferably includes from 1 to 10, and more preferably from 1 to 5 cycles of contact/amplification of the nucleic acids fixed onto the polypeptide of the invention.

The nucleic acids selected for their specific fixation to a polypeptide according to the invention are then characterized, preferably by sequencing.

The invention also relates a kit or pack for screening a nucleic acid interacting with a polypeptide according to the invention, comprising:

a) a polypeptide according to the invention;

b) optionally, a statistical population of nucleic acids of 20 to 50 nucleotides in length.

The invention is further illustrated, without in any way being limited, by the figures and examples.

FIG. 1 illustrates the amino acid sequence of the RRS1-R polypeptide. The different characteristic domains are represented.

FIG. 2 illustrates the amino acid sequence of the RRS1-S polypeptide. The different characteristic domains are represented.

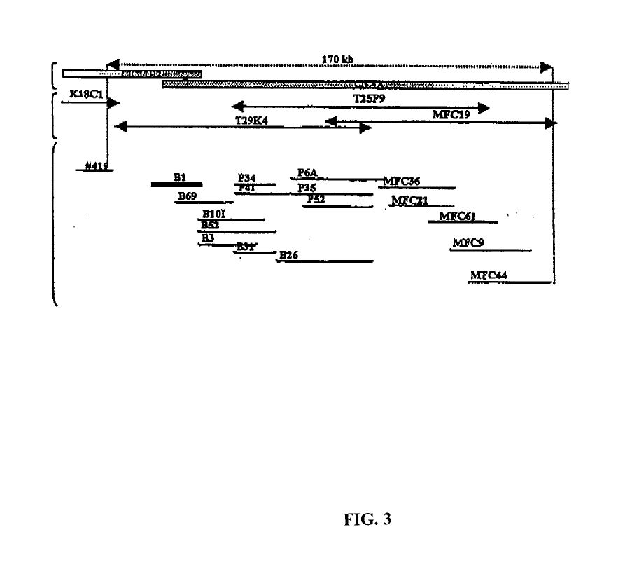

FIG. 3 illustrates the representation and location of the clones YACS, BACS and cosmids covering the genomic region of interest of A. thaliana.

### MATERIALS AND METHODS

a. Analysis by Southern Blot

The genomic DNA was isolated from leaves of plants and plantlets of Arabidopsis according to the technique described by Deslandes et al. (1998). The experiments of the Southern type were performed as described by Sambrook et al. (1989) using 2 μg of genomic DNA.

b. Extraction of the DNA from clones BAC, YAC, TAC, Cosmids, and from A. tumefaciens

The extraction of DNA from clones BAC and TAC was performed using a modification of the protocol supplied by the ABRC. 25 mL of a 12 hour culture of a BAC clone in the appropriate antibiotic were centrifuged for 5 min at 6 000 rpm. The sediment was resuspended in 4 mL of a buffer containing: 50 mM Glucose, 10 mM EDTA, 25 mM Tris, pH 8.0, 5 mg/mL of lyzozyme. After 5 min of incubation at ambient temperature, the cells were lysed by addition of 8 mL of 0.2 N NaOH, 1% SDS. A further incubation of 5 min at ambient temperature was followed by the addition of 6 mL potassium acetate 3M, pH 4.8. The mixture was incubated at 0° C. for 10 min., then centrifuged 15 min at 13 000 rpm. The supernatant was recovered, precipitated by addition of 0.7 volume of isopropanol, then centrifuged 15 min at 13 000 rpm. The sediment obtained was washed with ethanol 70%, dried 15 min at ambient temperature then resuspended in 0.6 mL of TE buffer (10 mM tris, pH 8, 1 mM EDTA).

The purification of the cosmid DNA was performed according to the protocol of Sambrook et al. (1989).

The extraction of the DNA from A. tumefaciens was performed following a protocol developed in the laboratory. 2 mL of a 12 hour culture in L medium supplemented with the appropriate antibiotics were centrifuged for 4 min at 13 000 rpm. The sediment was resuspended in 700 μl of buffer CTAB (100 mM Tris HCl, pH 8, 1.4M NaCl, 20 mM EDTA, pH 8, 2% w/v CTAB (hexadecyltrimethylammonium bromide) to which was added extemporaneously 0.07N of B-mercaptoethanol. The mixture was incubated for 10 min at 65° C., then extracted twice with an equal volume of phenol/chloroform (1V/1V) followed by an extraction with chloroform. The aqueous phase was then precipitated by 0.7 volume of isopropanol, the mixture centrifuged 10 min at 13 000 rpm and the sediment obtained was then resuspended in 50 μL of distilled water.

c. Plant Material and Protocol of Inoculation of Arabidopsis by R. solanacearum

The growth of Arabidopsis plants and the conditions of inoculation of these plants by different strains of R. solanacearum have been described by Deslandes et al. (1998).

d. Analysis of the DNA Sequences

The BLAST research programme (Altschul et al. 1990, 1997) was used for sequence analysis and comparison in the databases of Genbank, EMBL and Swissprot and in the databases on Arabidopsis (AatDB Database Arabidopsis).

### EXAMPLE 1

### Positional Cloning of RRS1

This cloning was performed on the sensitive ecotype Col-5 which contained the allele RRS1. The resistance to the strain GMI1000 of Ralstonia solanacearum was provided by the allele rrs1 of the resistant ecotype Nd-1.

A series of overlapping clones YACs (Yeast Artificial Chromosome) constructed by the Japanese consortium in charge of the programme of sequencing the chromosome V of Arabidopsis thaliana (http://www.kazusa.orjp/arabi/chr5/pmap/P1_map_{—}16.html) was obtained from the ABRC (Arabidopsis Biological Resource Center, Ohio State University, USA). RFLP markers with a polymorphism between the parent ecotypes Col-5 and Nd-1 were generated from the left and right ends of inserts of the YACs clones following the protocol of Schmidt et al. (1996). These different markers, EW7G12LE (LE, Left End: generated from the left end of the insert of the YAC), 11H2RE (RE, Right End: generated from the right end of the insert of the YAC), 11H2LE, 4H11RE as well as a marker T43968 (clone of cDNA positioned on the YAC CIC4E12 and obtained from the ABRC) were used as RFLP markers so as to localize more precisely the locus RRS1. Two of these markers (T43968 and EW7G12LE) were shown to be polymorphic with the parent ecotypes and allowed restriction of the region containing the RRS1 locus to a region of about 600 kb. On the 18 remaining recombinant plants, only 11 lines still showed a recombination event between these 2 markers and the RRS1 locus.

In addition, the markers generated from the ends of the YACs clones were used to perform the screening of a TAMU bank (Texas A & M University) of BACs (Bacterial Artificial Chromosome), a bank obtained through the ABRC. This screening, performed following conditions given by the ABRC, led to the isolation of a substantial number of BACs clones. Markers were then generated from the ends of some of these BACs by of “plasmid rescue” and inverse PCR techniques (Woo et al., 1994). These markers, 1H19LE, 2G14LE, 21F10LE, 9N23LE, 27P17LE and 29D23L E were used to position the different selected BACs with respect to each other (by digestion of the DNA of the different BACs and hybridization with each of the isolated ends). These data could be used to construct a series of overlapping BACs clones. Finally, we made use of the existence of a series of overlapping TACs (Transformation Artificial Chromosome) clones generated by the Japanese consortium. The corresponding TACs clones (K9E15, K18C1, K15122, K2N11) and the MFC19 clone (bank P1, Liu et al., 1995) were obtained by the ABRC.

In parallel with these studies, the number of lines showing a recombination event being relatively low, 650 F2 plants originating from a cross between the 2 parent ecotypes were tested so as to characterize other lines showing such an event between the T43968 and EW7G12LE markers and the RRS1 locus. With this aim, the DNA of several leaves of each F2 plant was extracted (Deslandes et al., 1998) and digested by the restriction enzyme BglII which allowed observation of a polymorphism with the T43968 and EW7G12LE markers on the 2 parental genotypes. The F2 plants thus selected were then self-fertilized and the seeds obtained (F3) were sown. The phenotype of these third generation plants was then determined by inoculation of the strain GMI1000. This approach enabled recombinant 15 F3 families between the T43968 and EW7G12LE markers to be obtained.

The markers corresponding to the ends of BACs were then used as RFLP markers on the 11 RILs lines and on the 15 F3 families. The sub-cloning of BACs and of cosmids by different restriction enzymes moreover allowed generation of other RFLP markers (Table 5).

TABLE
RFLP Markers used to map the RRSI locus over 15 F3 families
originating from the Col-5 and Nd-1 crossing.

| Markers | 18 | 32 | 149 | 195 | 251 | 324 | 328 | 332 | 364 | 380 | 401 | 428 | 446 | 540 | 566 |
|---|---|---|---|---|---|---|---|---|---|---|---|---|---|---|---|
| T43968 | AB | A | AB | A | AB | AB | A | AB | A | AB | B | AB | A | AB | AB |
| 1H19 n° 1 | | AB | AB | A | AB | AB | A | AB | AB | AB | B | AB | AB | AB | AB |
| #419 | | AB | A | A | AB | AB | A | AB | AB | AB | B | AB | AB | AB | AB |
| B1 | B | AB | A | — | AB | AB | A | AB | AB | AB | B | AB | AB | AB | AB |
| B1 n° 4 | — | AB | A | A | AB | AB | A | AB | AB | — | B | AB | AB | AB | AB |
| #6.1 | — | AB | A | A | AB | AB | A | AB | AB | AB | B | AB | AB | AB | AB |
| RRS1 | B | AB | A | — | AB | AB | A | AB | AB | AB | B | AB | AB | AB | A |
| MFC5 | — | — | — | — | — | AB/A | AB/A | — | — | AB | AB | — | — | — | — |
| MFC61 | B | AB | A | A | — | A | AB | B | — | AB | AB | — | — | B | — |
| MFC14 | — | — | — | — | — | A | AB/B | — | — | AB/B | — | — | — | — | A |
| 9N23 N° 2 | B | AB | A | A | AB | — | AB | AB | — | B | AB | AB | AB | AB | — |
| 9N23 N° 18 | B? | AB | A | — | — | — | — | — | — | — | — | A | — | — | — |
| 2G14LE | B | AB | A | A | AB | — | AB | B | — | B | AB | — | — | B | — |
| EW7G12LE | B | AB | A | AB | B | B | AB | B | AB | B | AB | A | AB | B | A |

For each F3 family, the genotype revealed by the use of the different RFLP markers is shown in this table (A: Col-5 genotype, B: Nd-1 Genotype, AB: heterozygous genotype).
T43968: marker obtained from the ABRC (Ohio, USA).
1H19 n° 1, , 9N23 N° 2, 9N23 N° 18: fragments of the clones BACs T1H19 and T9N23 (digestion BamHI-XhoI) cloned in the vector pKS (Stratagene, USA).
#419, B1: cosmids derived from the clones K18C1 and T29K4 respectively.
MFC5, MFC61 and MFC14: cosmids derived from partial BamHI digestion of the clone MFC19.
#6.1: phagemide derived from a phage bank Col-0 (zap, Stratagene, gift from Pr Lescure).
B1 n° 4: sub-clone of the cosmid B1
EW7G12LE: left end of the insert of the clone YAC EW7G12 (gift from W. Gassman).

These experiments enabled the RRS1 gene to be localized on 3 overlapping BACs clones (T29K4, T25P9 and MFC19), covering a region of about 170 kb (FIG. 3).

Contigs of cosmids covering the whole of the 2 BACs were obtained by partial digestion by the BamHI enzyme and sub-cloning of the DNA of the 2 BACs in the cosmid vector SLJ75515 (gift from Dr. J. Jones, The Sainsbury Laboratory). The use of these cosmids and of sub-clones of these cosmids obtained by digestion with the enzyme HindIII and insertion into the vector pBlueScript enabled RRS1 to be localized on a cosmid of 18 Kb, the cosmid B1 derived from the clone BAC T29K4. A bank of cDNA of the ecotype Col-0 prepared in the vector IZAP (gift from B. Lescure of our Institute) was then screened with the DNA of the insert of the cosmid B1 and enabled isolation of several clones of cDNA whose nucleotide sequence was determined. At the same time, the nucleotide sequence of a TAC clone, K9E15 which covers the cosmid B1, was added to the data banks by the Japanese consortium. Some cDNA clones whose nucleotide sequence was known could thus be localized on the cosmid B1. One of these, named #6.1, used as RFLP markers as well as a CAPS marker, B69.1, enabled delimitation of a region of about 10 kb of the cosmid B1 containing the RRS1 locus. Given the sequence of the cosmid B1, it was possible to generate several pairs of primers so as to identify the PCR markers of the CAPS type. Two recombinant F3 plants, 324 and 566, showing a recombination event within the cosmid B1, allowed identification of the RRS1 gene.

### EXAMPLE 2

### Isolation and Characterization of RRS1

A bank of cosmids prepared from the DNA of the ecotype Nd-1 was constructed in the vector SLJ75515 by the group of J. Beynon (Horticultural Research International, Wellesbourne, GB). This bank was kindly given to us and its screening was performed (according to the hybridization conditions of the Southern type described by Deslandes et al., 1998) using the DNA of the clone T1H19 which contains the RRS1 locus. Several cosmid clones were isolated and characterized. One of these, the clone H, was selected since it showed a restriction profile identical with that of B1, the cosmid on which RRS1-S had been localized by positional cloning. The sequence of the RRS1-S gene being known, it was possible to generate oligonucleotide primers which enabled the nucleotide sequence of the region corresponding to RRS1 of the cosmid H to be determined. The determination of the nucleotide sequence was performed on a Perkin-Elmer 373 sequencer (kit Dye Terminator) and the different sequences were assembled using the “Staden” programs for sequence assembly, available on a UNIX station (Roger Staden, MRC, Cambridge, UK). The different primers used were the primers of the “K” series, i.e. all the primers whose names begin with the letter “K” (see Table 6).

### EXAMPLE 3

### Complementation of the Sensitive and Resistant Ecotypes by RRS1-R and RRS1-S

a. Constructions

The cosmid vector SLJ 75515 of clones H and B1 has the advantage of being directly transferable to Agrobacterium tumefaciens. This transfer of cosmids of E. coli (strain XL1Blue for the cosmid B1 and strain DH12S for the cosmid H) into A. tumefaciens (strain GV3 101 containing the helper plasmid pMP90) was performed by triparental conjugation using a third strain, pRK2013 (E. coli) following the protocol of Tolmasky et al. (1984). The colonies of A. tumefaciens containing the cosmids H or B1 were selected on an L agar medium supplemented with tetracycline (10 μg/ml) and gentamycin (25 μg/ml). The dishes were then incubated at 28° C. for 3 days.

The presence of the cosmid DNA was checked by PCR amplification using specific primers of inserts of the clones H and B1 (pair RT1/RT2 for example). In addition, experiments of the Southern type enabled the stability of the cosmid DNA in A. tumefaciens to be verified.

The insert of the cosmid B1 was sub-cloned using the enzyme BamH1 which generated two fragments: 9.68 and 8.03 kb. For the cosmid H, 2 BamHI fragments of comparable size to those of the cosmid B1 were obtained. The BamHI site was only present once in the genomic sequences of RRS1-R and RRS1-S. Each of the 2 sub-fragments derived from the cosmids B1 and H was introduced into the binary vector pDHB321.1 supplied by Dr. Bouchez (INRA Versailles). After transformation in a strain of E. coli (XL1BlueMR, Stratagene, USA), the plasmid DNA was extracted (Maniatis 1978). The strain of A. tumefaciens GV3101 was then transformed by the heat shock technique (Holsters et al., 1978). The colonies of A. tumefaciens containing the sub-clones of the cosmids B1 and H were then selected on L agar medium containing gentamycin (25 μg/ml) and Kanamycin (50 μg/ml). The dishes were then incubated at 28° C. for 48 h.

The presence of the plasmid DNA was checked by PCR amplification using specific primers of the 2 sub-fragments of the clones H and B1. In addition, experiments of the Southern type enabled the stability of the plasmid DNA in A. tumefaciens to be verified. These constructions were then introduced into the genome of the plants Col-5 and Nd-1.

b. Transformation of the Ecotypes Nd-1 and Col-5 by Agrobacterium tumefaciens

In order to demonstrate the function of the candidate genes, complementation experiments of the sensitive and resistant phenotypes by the cosmids H and B1 were performed. The conventional techniques of transformation of Arabidopsis by A. tumefaciens (Clough and Bent 1999) were used to generate the transgenic plants described in this work. The seeds of plants transformed by A. tumefaciens (generation T1) were planted in pots containing a layer of perlite (about 2 cm) covered with a layer of sand of the same thickness soaked with a solution of the gluphosinate herbicide (trade name: BASTA) at a concentration of 15 mg/L. The pots were then placed for 4 days in the dark at 4° C., then transferred to long-day conditions (16 h of day/8 h of night). After about 10 days, the resistant plants were selected and repotted individually. The T2 progeny of each transgenic T1 plant obtained by self-fertilization was used for the experiments of inoculation by the pathogenic agent.

c. Results Obtained

c.1 The RRS1-R gene provided resistance to strain GMI1000 of Ralstonia solanacearum.

Complementation experiments performed using the cosmid H containing the RRS1-R allele introduced into the ecotype Col-5 were carried out. After selection by the herbicide BASTA, 12 transgenic plants could be isolated (lines numbered CH1.1 to CH1.12). These plants were self-fertilized so as to obtain the T2 progeny of each of them and 24 individuals of each transgenic line obtained were inoculated with strain GMI1000 of R. solanacearum. While the control plants (Col-5) had wilted 7 days after inoculation with the pathogen, the transgenic plants containing the RRS1-R allele of Nd-I showed no symptoms of wilting. These results show that the RRS1-R gene can provide resistance to the strain GMI1000 of R. solanacearum in plants with genetic base Col-5. The expression of RRS1-R could lead to a reduction of the bacterial multiplication in the infected plant, which could explain the absence of symptoms.

c.2 The RRS1-R Gene Provided Resistance to Different Strains of Ralstonia solanacearum.

Some strains of R. solanacearum induce responses (disease or absence of symptoms) identical to those of the strain GMI1000 in the ecotypes Nd-1 and Col-5. These isolated strains of very diverse plant-hosts (cf. table) originate from different regions of the globe. The capacity of the RRS1-R gene to provide resistance to these different strains was thus tested. Col-5 transgenic plants containing the RRS1-R gene were thus infected by these different strains. While the control plants (Col-5) had wilted 7 days after inoculation with the pathogen, the transgenic plants containing the RRS1-R allele of Nd-1 showed no symptoms of wilting. This result shows that the RRS1-R gene is able to provide resistance to different strains of R. solanacearum.

c.3 Does the RRS1-S Gene Provide Sensitivity to Strain GMI1000 of R. solanacearum?.

The response of Nd-1 plants transformed by the RRS1-S gene of the Col-5 sensitive ecotype was tested. 104 Plants were selected for their resistance to BASTA (B1.1 to B1.104) and self-fertilized. These transgenic plants were inoculated by the strain GMI1000. Only one transgenic line (B1.3) developed symptoms of wilting while the other lines showed no symptoms under conditions inducing complete wilting of control Col-5 plants.

### EXAMPLE 4

### Experiments of Complementation by Genomic Clones RRS1-S and RRS1-R.

A SphI digestion of the cosmid B1 gave a fragment of 9 393 pb (fragment between base 6557 and base 15950, based on the numbering of the sequence of the clone BAC K9E15 published by the Japanese consortium) covering the whole of the RRS1-S gene (promoter, introns/exons, polyadenylation signal sequence). This SphI fragment was ligated into a plasmid pUC19 digested by the SphI enzyme and dephosphorylated. This construction was then introduced into the strain DH5α of E. coli. This clone was defined as being the genomic clone RRS1 and was named B1puc3.

The same type of experiment was performed from the cosmid H. The genomic clone obtained was named Hpuc2.

In parallel, the binary vector pDHB321.1 was digested by the enzyme BamHI (unique cloning site). Ligation of the inserts (SphI/SphI) of the clones B1puc3 and Hpuc2 (derived from a SphI digestion) in the binary vector (BamHI/BamHI) thus required use of an adaptor so as to ligate the BamHI and SphI ends to each other. Two oligonucleotide primers were involved in the synthesis of this adaptor: it was composed of an equimolar mixture (5 μM) of the primers named “BamSph” (5′-GAT CGC GGC CGC CAT G-3′) and “Not” (5′-GCG GCC GC-3′). The mixture was subjected to a temperature of 95° C. for 3 minutes and then allowed to return to ambient temperature naturally.

To 100 ng of vector pDHB321.1 digested by the enzyme BamHI, 1 μl of the solution of the adaptor of 5 μM concentration was added. The ligation of the linearized vector and of the adaptor took place in a final volume of 10 μl for 12 hours at 4° C. The reaction was then stopped by dilution (addition of 20 μl of water). The excess of adaptor was then removed by passage of the ligation mixture over a microspin S400 column (Pharmacia, ref).

The DNA of the clones B1puc3 and Hpuc2 was digested by the enzyme SphI. After inactivation of the enzyme at 65° C. for 20 minutes, an aliquot of each digestion (about 100 ng) was used to perform the ligation with the vector pDHB321.1+adaptor. These constructions were then introduced into strain DH5α of E. coli and selected on an L agar medium containing 50 μg/ml of kanamycin (resistance provided by the binary vector): the clones which contained the insert derived from the clones B1 and H (about 9 400 pb) were named B1MT9 and HMTB respectively. The plasmid DNA extracted from these clones was then used to transform strain GV3101 of A. tumefaciens by heat shock. The colonies were selected on an L agar medium containing 25 μg/ml of gentamycin (resistance provided by the helper plasmid pMP90) and 50 μg/ml of kanamycin. The colonies of A. tumefaciens transformed by the DNA of the clones B1MT9 and HMTB were named B1MT9.1 and HMTB2 respectively.

### EXAMPLE 5

### Expression of the RRS1-S and RRS1-R Genes by Experiments of the Northern Type

The expression of the RRS1-S and RRS1-R genes was studied in non-infected plants by experiments of the Northern type. After extraction of the total RNA by the technique described by Lummerzheim (1993), the polyadenylated RNAs were purified by use of the Dynabeads Kit (Dynal, USA). After determination at 260 nm, the mRNAs were deposited on gel under denaturing conditions (Marco et al. 1990), then transferred onto a membrane of nitrocellulose (Hybond N+, Amersham Pharmacia Biotech). The hybridization was performed following the protocol given by the supplier.

### EXAMPLE 6

### Production of Full-Length cDNA Clones by RACE PCR

The production of full-length cDNA clones required use of a RACE (Rapid Amplification of cDNA Ends) PCR kit. The details of the experimental protocols given below are valid for the clones of cDNA derived from the ecotypes Col-5 and Nd-1.

a. RACE PCR (Rapid Amplification of cDNA Ends)

The 5′ and 3′ ends of the cDNA clones were generated by RACE PCR by use of the kit “Smart Race cDNA Amplification” (Clontech, USA). The total RNAs were extracted from two-week-old plantlets of the ecotypes Col-5 and Nd-1 following the protocol of Lummerzheim et al. (1993). The reverse transcriptase reactions were performed on 1 μg of total RNA following the conditions given by the manufacturer. The first strands of the cDNA were synthesized using the following primers:

5′CDS and SMART II oligo (Clontech) for obtaining the 5′ ends

3′CDS (Clontech) for obtaining the 3′ ends

The oligonucleotide primers used for the amplification of the 5′ ends of the cDNA RRS1 (Col-5) and rrs1 (Nd-1) were:

the universal primer UPM (Universal Primer Mix, kit Smart Race)

the antisense primer RT4, specific for the cDNA RRS1-S/RRS1-R (see table 2).

The oligonucleotide primers used for the amplification of the 3′ ends of the cDNA RRS1 (Col-5) and rrs1 (Nd-1) were:

the universal primer UPM (Universal Primer Mix, kit Smart Race)

the primer RT6, specific for the cDNA RRS1-S/RRS1-R (see table 2).

The RT4 and RT6 primers were selected so that the amplification products of the 5′ and 3′ ends had in common a region of 500 pb containing a unique BamHI restriction site.

The conditions of the amplifications of the 5′ and 3′ ends were the following:

5 cycles (94° C. for 30 seconds, 72° C. for 5 minutes)

5 cycles (94° C. for 30 seconds, 70° C. for 30 seconds, 72° C. for 5 minutes)

30 cycles (94° C. for 30 seconds, 68° C. for 30 seconds, 72° C. for 5 minutes)

The size of the amplification products of the 5′ and 3′ ends were 3 708 pb and 1 230 pb for Col-5; 3 714 and 1 231 pb for Nd-1 respectively. These PCR products were cloned in the vector pGemT-easy (Promega). The sequencing of these clones enabled precise determination of the beginning (5′) and end (3′) of the untranslated transcribed region of the cDNA clones derived from the ecotypes Col-5 and Nd-1. The size of the full-length cDNA clones derived from the plants Col-5 and Nd-i was respectively 4 336 and 4 343 pb.

b. Production of Full-Size cDNA Clones

Full-size cDNA clones were generated (i.e. 4 336 and 4 343 pb) by performing a double digestion BamHI (single site) and NotI (site present at the multiple cloning site of the vector pGemT-easy) on a mixture of 2 plasmids with the object of excising the inserts corresponding to the 5′ and 3′ ends. The 5′ and 3′ inserts were then ligated together in the plasmid pGemT-easy digested by NotI and dephosphorylated. After transformation of bacteria DH5α with the ligation mixture and spreading on selective medium (medium L+ampicillin 50 μg/mL+0.5 mM IPTG+80 μg/mL X-Gal), the positive clones containing the full-size cDNA clone were selected by performing a PCR screening on colonies using primers K3.5 and RT2 (see table 6) which are specific to each of the 5′ and 3′ ends respectively.

A second RACE PCR experiment was performed on the first strands of cDNA produced by the reverse transcriptase reaction described above with the object of introducing two SalI restriction sites, one being located just upstream of the initiation codon ATG (point of initiation of the translation), the other being located after the polyadenylation signal. The two pairs of primers used to amplify the 5′ and 3′ ends were 5′RRSalI/RT8 and 3′RRSalI/RT7 respectively.

The amplification conditions, the cloning of the PCR products and the method of obtaining the full-length cDNA clones were the same as those described above where the unique restriction site common to the 5′ and 3′ ends was a AflII site.

The introduction of SalI sites on either side of the sequence of the cDNA clone was in order to facilitate its cloning in different types of vectors.

### EXAMPLE 7

### Domain Exchange Experiment by Production of Chimeric RRS1-S/RRS1-R Clones

The plasmid DNA used to perform the domain exchange experiments originated from the genomic clones B1puc3 and Hpuc2 described above. These 2 plasmids were digested by the enzyme BamHI whose restriction site was localized in the region showing similarities with a resistance gene (5th exon). This digestion generated 2 fragments, one of 4 881 pb (clone B1puc3 containing the RRS1-S gene) containing the 5′ portion of the genomic clone (promoter and coding portion of the RRS1-S gene up to the level of the 5th exon) and a fragment of 7 180 pb (clone B1puc3) containing the 3′ portion of the genomic clone (4 512 pb containing the end of the region showing similarities with the resistance gene, the region similar to the gene coding a transcription factor of type WRKY and the 3′ untranslated transcribed region; 2 668 pb of plasmid vector pUC19).

The size of the fragments generated by the BamHI digestion of the plasmid Hpuc2 was of the same order of magnitude as that of the fragments obtained by BamHI digestion of the plasmid B1puc3.

After digestion by the BamHI enzyme, an aliquot of each of the DNA fragments generated was dephosphorylated. The residue was deposited on gel so as to purify the fragments corresponding to the 5′ portion of each genomic clone. The fragment of DNA corresponding to the 5′ portion of the RRS1-S gene was then ligated with the 3′ portion of the RRS1-R gene. Similarly, the fragment of DNA corresponding to the 5′ portion of the RRS1-R gene was ligated with the 3′ portion of the RRS1-S gene. These constructions were then introduced into E. coli (strain DH5α) and were respectively named 3BH (5′RRS1-S/3′RRS1-R) and 6HB (5′RRS1-R/3′RRS1-S). Specific primers of the 5′ and 3′ regions of the RRS1-S and RRS1-R genes were used to verify these constructions. The DNA of each plasmid was purified, digested by SphI, an enzyme allowing the excision of the complete genes. The inserts were then ligated into the plasmid vector pDHB321.1 digested by the enzyme BamHI and whose ends had been rendered compatible with the SphI ends using an adaptor (see above). These constructions were successively introduced into strain DH5α (the clones were named 3BHMT7 (5′RRS1-S/3′RRS1-R) and 6HBMT2 (5′RRS1-R/3′RRS1-S)) then into strain GV3101 of A. tumefaciens (clones named 3BHMT7.1 and 6HBMT2.1).

### EXAMPLE 8

### Search for Proteins Interacting with Proteins rrs1 and RRS1 by the Double Hybrid Technique in Yeast

In order to identify the proteins interacting with the RRS1-S and RRS1-R proteins, the double hybrid system in yeast was used (kit Clontech, Matchmaker Gal4 Two-Hybrid System 3). The complete cDNA clones corresponding to the RRS1-S and RRS1-R genes and the sub-fragments corresponding to certain specific domains of these genes (domains TIR, NBS, LRR, WRKY) were used to screen cDNA banks with the object of isolating genes whose products interacted with the products of these DNA fragments.

### EXAMPLE 9

### Modulation of the Expression of the RRS1-S and RRS1-R Genes by Sense and Antisense Approaches

For the antisense approaches, two different constructions were performed.

The first consisted of a fragment of 299 pb (position 3 642 to 3 941 of the sequence SEQ ID N^{o} 8) fragment corresponding to the domain WRKY of the protein RRS1-1. PCR primers (B1AS5′ and B1AS3′ which allowed the introduction of a NcoI and SalI site respectively) were generated to amplify this DNA fragment. The amplification was performed on single-strand cDNA resulting from the “reverse transcription” of total RNA of leaves of two-week old Col-5 plants. The polymerase used was Deep Vent (Biolabs, New England) and the conditions of amplification were those described by the manufacturer. The amplification product B1AS5′/B1AS3′ was digested by the enzymes NcoI and SalI and the digestion product deposited on gel and purified (Kit Jetsorb, Quantum Appligene). This DNA fragment was then ligated into the vector PA1 proterm 2 (gift from Pr. Lescure, our Institute) digested by the enzymes NcoI and SalI. This construction was introduced into the strain DH5α of E. coli. The corresponding plasmid DNA was purified, digested by the enzyme BamHI, which allowed the excision of an insert containing the promoter of the gene EF1a, the 299 pb of the RRS1-S gene in antisense orientation and the terminator of the gene EF1a (Axelos et al., 1989). This BamHI fragment was then inserted by ligation into the binary vector pDHB321.1 digested by BamHI. This new construction was introduced into strain DH5α of E. coli. The purified plasmid DNA was used to transform strain GV3101 of A. tumefaciens.

The second antisense construction was performed by excising an EcoRI fragment of the insert of the clone of full-length RRS1-R cDNA (sequence SEQ ID N^{o} 4). The DNA fragment thus generated (size about 3 930 pb) was introduced into the vector pKMB (Mylne and Botella, 1998) digested by EcoRI. This vector contained the 35S promoter of the cauliflower mosaic virus (CaMV 35S) and the corresponding terminator. This construction was successively introduced into the strains DH5α of E. coli and GV3 101 of A. tumefaciens by triparental conjugation.

The strains of A. tumefaciens obtained (29B iASMT2: fragment of 299 pb and J1: fragment of 3 930 pb) were used for the transformation of the ecotypes Nd-1 and Col-5.

TABLE 6

| | | |
|---|---|---|
| SEQ ID | Oligonucleotides | Sequence(5′ --3′) |

| | | |
|---|---|---|
| SEQ ID no 11 | K0 | AGC TCG AGA CTA TTC AGG |
| SEQ ID no 12 | K1.3 | AAA CAC TGA TAG CTA ACG G |
| SEQ ID no 13 | K1.5 | ATC TCT AAC GGT GGA TGG |
| SEQ ID no 14 | K2.3 | TGC ATT CAA GAC CTC TAG G |
| SEQ ID no 15 | K2.5 | CCG TTA GCT ATC AGT GTT T |
| SEQ ID no 16 | K3.3 | AGT CAT CAA GTG ACC ATC |
| SEQ ID no 17 | K3.5 | CTA GAG GTC TTG AAT GCA C |
| SEQ ID no 18 | K4 | GCA TCA CAG TAG TCC TCG |
| SEQ ID no 19 | K5 | ACA TCC AAG TCA ATA CCG G |
| SEQ ID no 20 | K6 | GCC AAT AGA GAT GTA CCA |
| SEQ ID no 21 | K7 | TGG TAC ATC TCT AAT GGC |
| SEQ ID no 22 | K8 | AGT AAC ACG TAA TGT AAC C |
| SEQ ID no 23 | K9 | ACC AGC AAG TTT AGG ATG A |
| SEQ ID no 24 | K10 | GAT GGT CAC TTG ATG ACT |
| SEQ ID no 25 | K11 | GGT GTA CAT AAA TCC TTG G |
| SEQ ID no 26 | K12 | CCA AGG ATT TAT GTA CAC C |
| SEQ ID no 27 | K13 | ACT CTT ATG GAG ATG CTC |
| SEQ ID no 28 | K14 | CGC ATC CTT AAA CTA CTG |
| SEQ ID no 29 | K15 | ATA TCT CCG GTT TCA ACC |
| SEQ ID no 30 | K16 | CCT TGG TGA GTA GCT CAC |
| SEQ ID no 31 | K17 | CCA TAG ATC TCC CTC GTC |
| SEQ ID no 32 | K18 | CCT TAT AGA ACT TCT CTC C |
| SEQ ID no 33 | K19 | CTC TTC GAG TGC ATC AGG |
| SEQ ID no 34 | K20 | CCG GTA TTG ACT TGG ATG T |
| SEQ ID no 35 | K21 | AGA TAC ACG TAC ACT GGC |
| SEQ ID no 36 | K22 | TCC AGC CCA GAT ATC AGG |
| SEQ ID no 37 | K23 | TGC ATA GGA AGC TTC TCC |
| SEQ ID no 38 | K24 | TTC AGA GGA ACT TGA GCG |
| SEQ ID no 39 | K25 | CCA AGC AAA TAA GCT TCC C |
| SEQ ID no 40 | K26 | ATC GTC CTC AAC ATC TCC |
| SEQ ID no 41 | K27 | AGG CGC AGA AGA CTG TGG |
| SEQ ID no 42 | K28 | TTG ATG CTC CAA GGT TCC |
| SEQ ID no 43 | K29 | GAG ATG TGT ACG AGA CGC |
| SEQ ID no 44 | K30 | CAA TCT CCA GCA GCT TCG |
| SEQ ID no 45 | K31 | TTG AGT GGT TGA ATG TCC |
| SEQ ID no 46 | K32 | CAC ACG AAT TCC TCA TCC |
| SEQ ID no 47 | K33 | TGA AGG AAC ACT CGT TGC |
| SEQ ID no 48 | K34Nd | GTC TTT CAG AGG CCT CGA |
| SEQ ID no 49 | K35Col | GGT AAG CAA TCT CTG ATA |
| SEQ ID no 50 | RT1 | ATG TTA TAT CGA CGT TGG |
| SEQ ID no 51 | RT2 | GAG GAA GTG GAA CGA GTG |
| SEQ ID no 52 | RT3 | AAC TCC TCC ATG TCC GTC |
| SEQ ID no 53 | RT4 | ATC TCC CTC GTC TAT AGC CGG TAT GG |
| SEQ ID no 54 | RT5 | GAT CAG GCT TCC GGG TCC TAG CCA GTC |
| SEQ ID no 55 | RT6 | AGT GAT GTC AAC ATG CGC CCA AGT ACC |
| SEQ ID no 56 | RT7 | AAC CTT CAA ACG TGC TCA GGG CTC TG |
| SEQ ID no 57 | RT8 | ACA TCT CCA GGT TCT TGG TTC CAC CC |
| SEQ ID no 58 | 5′RRSalI | GTC GAC ATG ACC AAT TGT GAA AAG GAT GA |
| SEQ ID no 59 | 3′RRSalI | GTC GAC CTT GTC TTG CAG TGA TGA GAG |
| SEQ ID no 60 | B1AS5′ | CTA TTC CAT GGA GGA GGA AGT GG |
| SEQ ID no 61 | B1AS3′ | TTA GTC GAC GAA GAA GAA ACA TAG |

### BIBLIOGRAPHIC REFERENCES

AXELOS, M., Bardet, C., Liboz, T., Le Van Thai, A., Curie, C. and Lescure, B. 1989. The gene family encoding the Arabidopsis thaliana translation elongation factor EF-1a: molecular cloning, characterization and expression. Mol. Gen. Genet. 219, 106-112.

ALSCHUL, S. F., Gish, W, Miller, W., Myers, E. W., and Lipman, D. 1990. Basic local alignment search tool. J. Mol. Biol. 215, 403-410.

ALSHUL S. F et al., (1997), Nucleic Acids Research, vol. 25: 33896-3402.

AUSUBEL et al., (1989), Current Protocols in Molecular Biology, Green Publishing Associates and WLILEY Interscience, New-York.

AUSUBEL et al. Eds., John WILEY and BROWN E. L., (1979), Methods Enzymol vol. 68: 109-151,

BEAUCAGE et al., (1981), Tetrahedron Lett., vol. 22: 1859-1862

BOUCHER, C. A., Barberis, P. A., Trigalet, A. P. and Demery, D. A. 1985. Transposon mutagenesis of Pseudomonas solanacearum: isolation of Tn5-induced avirulent mutants. J. Gen. Microbiol. 131, 2449-2457.

BECHTOLD N., ELLIS J., and PELLETIER G (1993) C.R. Acad. Sci. Pris 316, 1194-1199.

BROWN E L. et al., (1979) Methods Enzymol., 68: 109-151.

BEVAN et al., Nucleic Acids Research, 12: 8711-8721.

BOUCHER et al., 1993.

CHEN W., CHAO G., SINGH K B (1996). Plant J. 10, 955-966.

CLOUGH, S., and Bent, A. 1999. Floral dip: a simplified method for Agrobacterium-mediated transformation of Arabidopsis thaliana. Plant J. 16, 735-743.

CHO R. J. et al. (1998), Proc. Natl. Acad. Sci. USA, Vol.95 (7):3752

CORMACK B, VALDIVIA R. H. FALKOW S. 1996. Gene 173, 33-38.

DESLANDES, L., Pileur, F., Liaubet, L., Camut, S., Can, C., Williams, K., holub, E., Beynon, J., Arlat, M. and Marco, Y. 1998. Genetic characterization of RRS1, a recessive locus in Arabidopsis thaliana that confers resistance to the bacterial soilborne pathogen, Ralstonia solanacearum.

FULLER S. A. et al. (1996), Immunology in Current Protocols in Molecular Biology.

FROMONT-RACINE M. et al. (1997), Nature Genetics, vol. 16 (3): 277-282.

GREEN et al., (1986), Ann. Rev. Biochem., vol. 55 : 569-597.

GRIFFIN et al. (1989), Science, vol. 245 : 967-971.

HOUBEN WEYL, (1974), In Methoder Organischen Chemie, E. Wunsch Ed. vol.15-1 and 15-II, Thieme, Stuttgart.

HAMMOND-KOSACK K. E. et al., (1997), Annu. Rev. Plant. Physiol. Plant Mol. Biol., vol. 48: 575-607

HAMES B. D. and HIGGINS S. J., (1985), Nucleic Acid Hybridization: The Practical Approach, HANES and HIGGINS Ed., IRL Press, Oxford.

HARPER J. W. et al. (1993), Cell, vol. 75 : 805-816,

HORSCH et al., (1984).

IZANT J J, WEINTRAUB H., (1984), Cell, Vol. 36 (4): 1007-1015

JEFFERSON R. A., KAVANACH, T. A., and BEVAN, M. W. (1987). Embo J. 3901-3907.

KELLER H., PAMBOUKDJIAN N, PONCHET M, POUPET A, DELON R. VERRIER J L. ROBY D. and RICCI P. (1999). Plant Cell 11, 223-235.

KOCH Y. (1977), Biochem. Biophys. Res. Commun. Vol. 74: 488-491.

KOHLER G. and MILSTEIN C., (1975) Nature, vol. 256: 495.

KOZBOR et al. (1983), Hybridoma, vol. 2 (1): 7-16.

LIU, Y. G., Mitsukawa, N., Vazquez-Tello, A. and Whittier, F. 1995. Generation of a high quality P1 library of Arabidopsis suitable for chromosome walking. Plant J. 7, 351,358.

LUMMERZHEIM, M., de Olivera, D., Castresana, C., Miguens F. C.,

LEBEL E. HEIFETZ P., THORNE L., UKNES S., RYALS, J. AND WARD E. (1998). Plant J. 16, 223-233.

LOUZADA, E., Roby, D., Van Montagu, M., Timmerman, B. 1993. Identification of compatible and incompatible interactions between Arabidopsis thaliana and Xanthomonas campestris pv campestris and characterization of the hypersensitive response. Mol. Plant Microbe Interact. 6, 532-544.

MARCO, Y., Raguch, F., Godiard, L. and Froissard, D. 1990. Transcriptional activation of 2 classes of genes during the hypersensitive reaction of tobacco leaves infiltrated with an incompatible strain of the phytopathogenic bacterium, Pseudomonas solanacearum. Plant Mol. Biol. 15, 145-154.

MYLNE, J., and Botella, J., R. 1998. Binary vectors for sense and antisense expression of Arabidopsis ESTs. Plant Molecular. Biology Reporter 16, p 257-262.

MARTINEAU et al., JOHNS P. WINTER G. (1998), J. Mol. Biol., vol.280 (1):117-127.

MERRIFIELD R B, (1965a), Nature, vol. 207 (1996: 522-523)

MERRIFIELD R. B., (1965b), Science, vol. 150 (693): 178-185.

McELROY D., ZHANG W, CAO J, WU R (1990) Isolation of an efficient actin promoter for use in rice transformation. Plant Cell 2, p 163-171.

NARANG S. A. et al., (1979), Methods Enzymol., vol.68, 90-98.

ODELL JT, NAGY F. CHUA N H. (1985). Identification of DNA sequences required for activity of the cauliflower mosaic virus 35S promoter. Nature 313, p. 810-812.

PRANDL R., KLOSKE E., SCHOFFL F. (1995). Plant Mol. Biol.28,73-82.

PEARSON, W. T. and Lipman D. J. (1988). Improved tools for biological sequence comparison. Proc. Natl. Acad. Sci. USA 85, 2444-2448.

ROSSI et al. (1991), Pharmacol. Ther., vol. 5O: 245-254

RIDDER R. et al., (1995), Biotechnology (NY), vol.13 (3): 255-260 ou encore des anticorps humanises

REINMANN K. A. et al., (1997), Aids, Res. Hum. Retroviruses, vol. 13 (11): 933-943

SCHMIDT, R. West., J. Cnops, G., Lov, K. Balestrazzi, A. Dean C. 1996 Detailed description of four YAC contigs representing 17 Mb of chromosome 4 of Arabidopsis thaliana ecotype Columbia. Plant J. 9, 5755-5765.

SAMBROOK J., FRITSCH E. F. and T. MANIATIS, (1989), Molecular Cloning: A Laboratory Manual 2nd ed. Cold Spring Harbor Laboratory, Cold Spring Harbor NEW YORK.

SCZAKIEL G. et al. (1995) Trends. Microbiol., vol. 3 (6): 213-217.

SANDEZ-PESCADOR R., (1988), J. Clin. Microbiol., vol. 26(10): 1934, 1938.

TOLMASKY, M., E. and Crosa, J., H. (1984). Molecular cloning and expression of genetic determinants for the iron uptake system mediated by the Vibrio anguillarum plasmid pJM1. J Bacteriol 160, p 860-866.

URDEA N S et al. (1988), Nucleic Acids Research, vol. 11: 4937-4957.

URDEA N S et al., (1991), Nucleic Acids Symp. Ser., vol. 24: 197-200.

WABIKO H. et al., (1986), DBA, vol. 5.(4): 305-314.

WOO, S.-S, Jiang J., Gill, B. S., Paterson, A. H. and Wing, R. A. 1994. Nuc. Acid Res. 22, p 4922-4931.

1. Nucleic acid containing at least 15 consecutive nucleotides of a nucleotide sequence coding for a protein of resistance of a plant to a pathogen, said protein comprising:
a) an N-terminal portion containing at least one amino acid sequence rich in leucine and at least one nucleotide-binding site; and
b) a C-terminal portion containing a DNA-binding domain, said binding domain comprising the amino acid sequence “WRKYGQK”, as well as a nucleic acid of complementary sequence.

2. Nucleic acid according to claim 1, characterized in that it has at least 40%, advantageously 60%, preferably 80% and more preferably 90% identity in nucleotides with the nucleotide sequence SEQ ID N^{o} 1, as well as a nucleic acid of complementary sequence.

3. Nucleic acid according to claim 2, characterized in that it has at least 40%, advantageously 60%, preferably 80% and more preferably 90% identity in nucleotides with a nucleotide sequence selected from among the following sequences:
a) the nucleotide sequence between the nucleotide in position 260 and the nucleotide in position 636 of the nucleic acid SEQ ID N^{o} 1;
b) the nucleotide sequence between the nucleotide in position 746 and the nucleotide in position 1856 of the nucleic acid SEQ ID N^{o} 1;
c) the nucleotide sequence between the nucleotide in position 1937 and the nucleotide in position 2236 of the nucleic acid SEQ ID N^{o} 1;
d) the nucleotide sequence between the nucleotide in position 2326 and the nucleotide in position 3249 of the nucleic acid SEQ ID N^{o} 1;
e) the nucleotide sequence between the nucleotide in position 3438 and the nucleotide in position 4291 of the nucleic acid SEQ ID N^{o} 1;
f) the nucleotide sequence between the nucleotide in position 5377 and the nucleotide in position 5499 of the nucleic acid SEQ ID N^{o} 1;
g) the nucleotide sequence between the nucleotide in position 6085 and the nucleotide in position 6532 of the nucleic acid SEQ ID N^{o} 1;
as well as a nucleic acid of complementary sequence.

4. Nucleic acid according to claim 2, characterized in that it has at least 40%, advantageously 60%, preferably 80% and more preferably 90% identity in nucleotides with a nucleotide sequence selected from among the following sequences:
a) the nucleotide sequence between the nucleotide in position 637 and the nucleotide in position 745 of the nucleic acid SEQ ID N^{o} 1;
b) the nucleotide sequence between the nucleotide in position 1857 and the nucleotide in position 1936 of the nucleic acid SEQ ID N^{o} 1;
c) the nucleotide sequence between the nucleotide in position 2237 and the nucleotide in position 2325 of the nucleic acid SEQ ID N^{o} 1;
d) the nucleotide sequence between the nucleotide in position 3250 and the nucleotide in position 3437 of the nucleic acid SEQ ID N^{o} 1;
e) the nucleotide sequence between the nucleotide in position 4292 and the nucleotide in position 5376 of the nucleic acid SEQ ID N^{o} 1;
f) the nucleotide sequence between the nucleotide in position 5500 and the nucleotide in position 6084 of the nucleic acid SEQ ID N^{o} 1;
as well as a nucleic acid of complementary sequence.

5. Nucleic acid according to claim 1, characterized in that it contains at least 15 consecutive nucleotides of the nucleotide sequence SEQ ID N^{o} 4, as well as a nucleic acid of complementary sequence.

6. Nucleic acid according to claim 1, characterized in that it has at least 40%, advantageously 60%, preferably 80% and more preferably 90% identity in nucleotides with the nucleotide sequence SEQ ID N^{o} 5, as well as a nucleic acid of complementary sequence.

7. Nucleic acid according to claim 6, characterized in that it has at least 40%, advantageously 60%, preferably 80% and more preferably 90% identity in nucleotides with a nucleotide sequence selected from among the following sequences:
a) the nucleotide sequence between the nucleotide in position 1184 and the nucleotide in position 1560 of the nucleic acid SEQ ID N^{o} 5;
b) the nucleotide sequence between the nucleotide in position 1670 30 and the nucleotide in position 278 of the nucleic acid SEQ ID N^{o} 5;
c) the nucleotide sequence between the nucleotide in position 2861 and the nucleotide in position 3160 of the nucleic acid SEQ ID N^{o} 5;
d) the nucleotide sequence between the nucleotide in position 3254 and the nucleotide in position 4171 of the nucleic acid SEQ ID N^{o} 5
e) the nucleotide sequence between the nucleotide in position 4360 and the nucleotide in position 5213 of the nucleic acid SEQ ID N^{o} 5;
f) the nucleotide sequence between the nucleotide in position 6302 and the nucleotide in position 6424 of the nucleic acid SEQ ID N^{o} 5;
g) the nucleotide sequence between the nucleotide in position 6953 and the nucleotide in position 7136 of the nucleic acid SEQ ID N^{o} 5;
as well as a nucleic acid of complementary sequence.

8. Nucleic acid according to claim 6, characterized in that it has at least 40%, advantageously 60%, preferably 80% and more preferably 90% identity in nucleotides with a nucleotide sequence selected from among the following sequences:
a) the nucleotide sequence between the nucleotide in position 1561 and the nucleotide in position 1669 of the nucleic acid SEQ ID N^{o} 5;
b) the nucleotide sequence between the nucleotide in position 2781 and the nucleotide in position 2860 of the nucleic acid SEQ ID N^{o} 5;
c) the nucleotide sequence between the nucleotide in position 3161 and the nucleotide in position 3253 of the nucleic acid SEQ ID N^{o} 5;
d) the nucleotide sequence between the nucleotide in position 4172 and the nucleotide in position 4359 of the nucleic acid SEQ ID N^{o} 5;
e) the nucleotide sequence between the nucleotide in position 5214 and the nucleotide in position 6301 of the nucleic acid SEQ ID N^{o} 5;
f) the nucleotide sequence between the nucleotide in position 6425 and the nucleotide in position 6592 of the nucleic acid SEQ ID N^{o} 5;
as well as a nucleic acid of complementary sequence.

9. Nucleic acid according to claim 1, characterized in that it contains at least 15 consecutive nucleotides of the nucleotide sequence SEQ ID N^{o} 8, as well as a nucleic acid of complementary sequence.

10. Hybridizing nucleic acid, under very strict hybridization conditions, with a nucleic acid according to any one of claims 1 to 9.

11. Hybridizing nucleotide probe, under very strict hybridization conditions, with a nucleic acid according to any one of claims 1 to 10.

12. Hybridizing nucleotide primer, under very strict hybridization conditions, with a nucleic acid according to any one of claims 1 to 10.

13. Nucleic acid according to claim 10, characterized in that it is selected from among the polynucleotides of sequences SEQ ID N^{o} 11 to 61.

14. Antisense nucleotide sequence containing at least 15 consecutive nucleotides of a nucleic acid according to any one of claims 1 to 10.

15. Recombinant vector, characterized in that it contains a nucleic acid according to any one of claims 1 to 10.

16. Recombinant vector according to claim 15, characterized in that it is a vector of functional expression in a plant host cell.

17. Recombinant vector according to one of claims 15 and 16, characterized in that it is a vector of fungal, bacterial or viral origin.

18. Host cell transformed with a nucleic acid according to one of claims 1 to 10 or with a recombinant vector according to one of claims 15 to 17.

19. Host cell transformed according to claim 18, characterized in that it is a cell of prokaryotic or eukaryotic origin.

20. Host cell transformed according to claim 19, characterized in that it is a cell of Agrobacterium tumefaciens.

21. Host cell transformed according to claim 19, characterized in that it is a plant cell of Arabidopsis thaliana, of rape, of corn or of tobacco.

22. Recombinant multicellular plant organism, characterized in that it contains at least one host cell transformed according to one of claims 18 to 21.

23. Plant transformed with a nucleic acid according to one of claims 1 to 10, a nucleotide sequence according to claim 13 or a recombinant vector according to one of claims 15 to 17.

24. Transformed plant containing, in a form integrated into its genome, a nucleic acid according to one of claims 1 to 10, a nucleotide sequence according to claim 13 or a recombinant vector according to one of claims 15 to 17.

25. Method for obtaining a transformed plant, characterized in that it comprises the following steps a) obtaining a transformed plant host cell according to one of claims 19 to 21;
b) regenerating an entire plant from the recombinant host cell obtained from step a);
c) selecting the plants obtained from step b) which have integrated a polynucleotide of interest selected from among the nucleic acids according to one of claims 1 to 10.

26. Method for obtaining a transformed plant, characterized in that it comprises the following steps:
a) obtaining a host cell of Agrobacterium tumefaciens according to claim 20;
b) transforming the plant by infection with the cells of Agrobacterium tumefaciens obtained from step a);
c) selecting the plants which have integrated a polynucleotide of interest selected from among the nucleic acids according to one of claims 1 to 10.

27. Method for obtaining a transformed plant, characterized in that it comprises the following steps:
a) transfecting a plant cell with a nucleic acid according to one of claims 1 to 10 or a recombinant vector according to one of claims 15 to 17;
b) regenerating an entire plant from the recombinant plant cells obtained from step a);
c) selecting the plants which have integrated the nucleic acid according to one of claims 1 to 10 or the recombinant vector according to one of claims 15 to 17.

28. Method for obtaining a transformed plant according to one of claims 25 to 27, characterized in that it additionally comprises the steps of:
d) crossing between themselves two transformed plants such as those obtained from step c);
e) selecting the plants homozygous for the nucleic acid of interest.

29. Method for obtaining a transformed plant according to one of claims 25 to 27, characterized in that it additionally comprises the steps of:
d) crossing a transformed plant obtained from step c) with a plant of the same species;
e) selecting the plants derived from the crossing of step d) which have retained the nucleic acid of interest.

30. Transformed plant such as that obtained according to the method according to any one of claims 25 to 29.

31. Seed of a transformed plant according to any one of claims 23, 24 and 29.

32. Seed of a plant whose component cells contain in their genome a nucleic acid according to one of claims 1 to 10.

33. Use of a nucleic acid according to one of claims 1 to 10 for the in vitro or in vivo expression of a protein selected from among the RRS1-S or RRS1-R proteins or of a peptide fraction of these.

34. Use according to claim 33, characterized in that it is an in vivo expression in a plant transformed with such a nucleic acid.

35. Use of a nucleotide sequence according to claim 14, or of a recombinant vector containing a nucleotide sequence according to claim 14, to inhibit or block the expression of the gene coding for the RRS1-S protein or for the RRS1-R protein.

36. Method for detecting a component nucleic acid of the RRS1-S or RRS1-R gene in a sample, comprising the steps of:
a) placing a probe or a number of probes according to claim 11 in contact with the nucleic acid which may be contained in the sample;
b) detecting any hybrid formed between the nucleic acid of the sample and the probe or probes.

37. Kit or pack for detecting a component nucleic acid of the RRS1-S or RRS1-R gene in a sample, comprising:
a) a probe or a number of probes according to claim 11;
b) optionally, the reagents necessary for the hybridization reaction.

38. Method for amplifying a component nucleic acid of the RRS1-S or RRS1-R gene in a sample, comprising the steps of:
a) Placing a pair of primers according to one of claims 12 and 13 in contact with the nucleic acid which may be contained in the sample;
b) performing at least one amplification cycle of the nucleic acid contained in the sample;
c) detecting any nucleic acid which has been amplified.

39. Kit or pack for amplifying a nucleic acid in a sample, comprising:
a) a pair of primers according to one of claims 12 and 13;
b) optionally, the reagents necessary for performing the amplification reaction.

40. Polypeptide coded by a nucleic acid according to any one of claims 1 to 10.

41. Polypeptide according to claim 40, characterized in that it comprises an amino acid sequence SEQ ID N^{o} 9 or a polypeptide having at least 40% identity in amino acids with the sequence SEQ ID N^{o} 9.

42. Polypeptide according to claim 40, characterized in that it comprises an amino acid sequence SEQ ID N^{o} 10 or a polypeptide having at least 40% identity in amino acids with the sequence SEQ ID N^{o} 10.

43. Polypeptide containing amino acid modifications of 1, 2, 3, 4, 5, 10 to 20 substitutions, additions or deletions of an amino acid compared to the amino acid sequence of a polypeptide according to one of claims 41 and 42.

44. Polypeptide containing at least 5 consecutive amino acids of a polypeptide according to one of claims 40 to 43.

45. Fusion polypeptide, characterized in that it comprises the N-terminal portion of the RRS1-S polypeptide fused with the C-terminal portion of the RRS1-R polypeptide.

46. Fusion polypeptide, characterized in that it comprises the N-terminal portion of the RRS1-R polypeptide fused with the C-terminal portion of the RRS1-S polypeptide.

47. Nucleic acid coding for a polypeptide according to one of claims 45 and 46.

48. Antibody directed against a polypeptide according to one of claims 40 to 46.

49. Method for detecting the presence of a polypeptide according to one of claims 40 to 44 in a sample, comprising the steps of:
a) placing the sample in contact with an antibody according to claim 48;
b) detecting any antigen/antibody complex formed.

50. Diagnostic kit or pack for detecting the presence of a polypeptide according to one of claims 40 to 44 in a sample, characterized in that it comprises:
a) an antibody according to claim 48;
b) optionally, the reagents necessary for detecting the antigen/antibody complexes formed.

51. Method for screening a candidate substance fixing to a RRS1-S or RRS1-R polypeptide, characterized in that it comprises the steps of:
a) preparing a polypeptide according to one of claims 40 to 44;
b) obtaining a candidate substance to be tested;
c) placing the polypeptide from step a) in contact with the candidate substance from step b);
d) detecting any complex formed between the polypeptide and the candidate substance.

52. Kit or pack for screening a candidate substance fixing to a RRS1-S or RRS1-R polypeptide, comprising:
a) a polypeptide according to one of claims 40 to 44;
b) optionally, the reagents necessary for detecting the complexes formed between the polypeptide and a candidate substance to be tested.

53. Method for screening a candidate substance fixing to a RRS1-S or RRS1-R polypeptide, characterized in that it comprises the steps of:
a) obtaining a first nucleic acid coding for a fusion protein comprising a part of the polypeptide of interest fused to the DNA-binding domain of a transcription factor such as Gal4;
b) obtaining a second nucleic acid coding for a fusion protein comprising the candidate substance fused to the transcription domain of a transcription factor such as Gal4;
c) producing a nucleic acid containing a nucleotide sequence coding for a detectable marker, placed under the control of a regulatory sequence recognized by a transcription factor such as Gal4;
the nucleic acids a), b) and c) being inserted into appropriate vectors; and
d) co-transfecting yeast cells simultaneously with said vectors;
e) detecting the expression of the nucleotide sequence coding for the detectable marker.

54. Kit or pack for screening a candidate substance fixing to the RRS1-S or RRS1-R polypeptide, comprising:
a) a first nucleic acid coding for a fusion protein comprising a part of the polypeptide of interest fused to the DNA-binding domain of a transcription factor such as Gal4;
b) optionally, a second nucleic acid containing a nucleotide sequence coding for a detectable marker, placed under the control of a regulatory sequence recognized by the transcription factor such as Gal4;
c) a third nucleic acid coding for a fusion protein comprising the candidate substance fused to the transcription domain of the transcription factor such as Gal4;

55. Substance able to fix to the RRS1-S or RRS1-R polypeptide, characterized in that it may be obtained by a method according to one of claims 51 and 53.

56. Method for screening a nucleic acid interacting with a polypeptide according to one of claims 40 to 44, comprising the steps of:
a) obtaining a statistical population of nucleic acids of 20 to 50 nucleotides in length;
b) placing the population of nucleic acids from step a) in contact with a polypeptide according to one of claims 40 to 44;
c) characterizing the nucleic acid or acids interacting with said polypeptide.

57. Kit or pack for screening a nucleic acid interacting with a polypeptide according to one of claims 40 to 44, comprising:
a) a polypeptide according to one of claims 40 to 44;
b) optionally, a statistical population of nucleic acids of 20 to 50 nucleotides in length.
